(19) United States
(12) Patent Application Publication
(10) Pub. No.: US 2026/0027068 A1
(43) Pub. Date: Jan. 29, 2026

(54) METHODS OF ADMINISTERING R-KETAMINE

(71) Applicant: Perception Neuroscience, Inc., New York, NY (US)

(72) Inventors: Maju MATHEWS, Voorhees, NJ (US); Terence Alfred KELLY, Ridgefield, CT (US); Mouhannad JUMAA, San Jose, CA (US)

(21) Appl. No.: 19/144,214

(22) PCT Filed: Dec. 26, 2023
(86) PCT No.: PCT/US2023/085915

§ 371 Date: Jun. 27, 2025

Related U.S. Application Data
(60) Provisional application No. 63/477,439, filed on Dec. 28, 2022.
(60) Provisional application No. 63/582,273, filed on Sep. 13, 2023.

(51) Int. Cl. A61K 31/135; A61K 9/00; A61P 9/02; A61P 25/24

(52) U.S. Cl. CPC A61K 31/135; A61K 9/0019; A61P 9/02; A61P 25/24

(57) ABSTRACT

The disclosure provides a method of treating or ameliorating depressive symptoms of a subject, comprising administering a therapeutically effective amount of a composition comprising R(−)-ketamine, or a pharmaceutically acceptable salt thereof, to the subject.

### CROSS REFERENCE TO RELATED APPLICATIONS

This application claims priority to U.S. Provisional Application Nos. 63/477,439, filed Dec. 28, 2022, and 63/582,273, filed on Sep. 13, 2023, the contents of each of which are incorporated by reference in their entireties herein.

### BACKGROUND

Racemic ketamine, and the S(+)-ketamine isomer, have been implicated in the treatment of a number of diseases and disorders that affect cognitive or neurological function, including depression, bipolar disorder, post-traumatic stress disorder, obsessive compulsive disorder, substance use disorders, Alzheimer's disease, Parkinson's disease, Lewy body dementia, and others. However, although racemic ketamine has been extensively used as an approved parenteral anesthetic, its approval for treating such disorders has been limited due to concerns over side effects and its potential for abuse. Similarly, approval of the S(+)-ketamine isomer for depression by administration of Spravato is limited to the clinic and under the observation of medical personnel.

There is thus a need for additional ketamine-like agents that reduce the side effects seen with racemic ketamine and S(+)-ketamine while providing efficacy for symptoms, and which have a convenient route of administration to permit the use outside of a supervised clinical setting. Accordingly, the present disclosure addresses this unmet need.

### SUMMARY

The disclosure provides methods of treating or ameliorating a depressive symptom of a subject, the methods comprising administering a therapeutically effective amount of a composition comprising R(−)-ketamine or a pharmaceutically acceptable salt thereof to the subject, wherein the composition is substantially free of S(+)-ketamine or a pharmaceutically acceptable salt thereof, and wherein the therapeutically effective amount comprises a maximum blood plasma concentration (Cmax) of R(−)-ketamine of at least 250 ng/mL.

In some embodiments of the methods of the disclosure, the depressive symptom is a symptom of a mood disorder in the subject. In some embodiments, the mood disorder comprises depression, optionally wherein the depression is treatment resistant depression or major depressive disorder. In some embodiments, the mood disorder comprises bipolar disorder, post traumatic stress disorder, obsessive compulsive disorder, autism spectrum disorder, schizophrenia, or dementia. In some embodiments, the depressive symptom is associated with a substance use disorder in the subject.

The disclosure provides methods of treating a disease or disorder in a subject, the methods comprising administering a therapeutically effective amount of a composition comprising R(−)-ketamine or a pharmaceutically acceptable salt thereof to the subject, wherein the composition is substantially free of S(+)-ketamine or a pharmaceutically acceptable salt thereof, and wherein the therapeutically effective amount comprises a maximum blood plasma concentration (Cmax) of R(−)-ketamine of at least 250 ng/mL.

In some embodiments of the methods of the disclosure, the Cmax is between 250 and 1000 ng/mL. In some embodiments the Cmax is between 250 and 800 ng/mL.

In some embodiments of the methods of the disclosure, the depressive symptom is assessed by a Montgomery Åsberg Depression Rating Scale (MADRS) subject score, and the MADRS subject score is decreased by administration of the composition. In some embodiments, the MADRS subject score is decreased by about 2 to about 20 by administration of the composition. In some embodiments, the MADRS subject score is decreased by about 2 to about 20 when measured about 24 hours, about 7 days, or about 14 days after administration of the composition begins. In some embodiments, the MADRS subject score is decreased by about 2 to about 20 after administration of the composition begins.

In some embodiments of the methods of the disclosure, the therapeutically effective dose of the composition comprises from about 50 mg to about 300 mg R(−)-ketamine or a pharmaceutically acceptable salt thereof. In some embodiments, the therapeutically effective dose of the composition comprises from about 50 mg to about 150 mg R(−)-ketamine or a pharmaceutically acceptable salt thereof.

In some embodiments of the methods of the disclosure, the composition comprises R(−)-ketamine hydrochloride.

In some embodiments of the methods of the disclosure, the composition is administered intravenously or subcutaneously. In some embodiments, the intravenous administration comprises intravenous infusion. In some embodiments, the composition is administered over a time period from about 10 minutes to about 1.5 hours via intravenous infusion. In some embodiments, the composition is administered once per day, one every other day, once every three days, once every four days, once every 5 days, once every 6 days, once per week, every other week, every 10 days, or once per month. In some embodiments, the methods comprise a dosing schedule comprising: (a) an initial period in which the composition is administered every 1, 2, 3 or 4 days; and (b) a maintenance period in which composition is administered less frequently than during the initial period. In some embodiments, the composition is administered once per week, twice per week, every other week, every 10 days, or once per month during the maintenance period.

In some embodiments of the methods of the disclosure, the composition further comprises a pharmaceutically acceptable carrier.

In some embodiments of the methods of the disclosure, the therapeutically effective amount of the composition comprising R(−)-ketamine does not cause significant dissociation, derealization or sedation in the subject. In some embodiments, the therapeutically effective amount of the composition increases systolic blood pressure of the subject by less than 40 mmHg, optionally less than 10 mmHg, as measured within 14 days of administration. In some embodiments, the therapeutically effective amount of the composition increases diastolic blood pressure of the subject by less than 25 mmHg, optionally less than 10 mmHg, as measured within 14 days of administration. In some embodiments, administration of the therapeutically effective amount of the composition has fewer side effects or adverse events than administration of a therapeutically effective amount of S(+)-ketamine or racemic ketamine. In some embodiments of the methods of the disclosure, the disease or disorder is a neurodegenerative disease or disorder, a neurodevelopmental disorder, an inflammatory or bone disease. In some embodiments, the neurodegenerative disease or disorder comprises Parkinson's disease, Parkinson's syndrome, Huntington's disease, spiny erythrocyte chorea, spinal cord cerebellar degeneration, amyotrophic lateral sclerosis, spinal muscular atrophy, primary lateral sclerosis, spinal and bulbar muscular atrophy, syringomyelia, neurospinous erythrocytosis, eating disorders, Alzheimer's disease, Lewy body dementia, basal ganglia degeneration, multiple sclerosis, traumatic brain injury, cerebral infarction, or cardiovascular disease. In some embodiments, the neurodevelopmental disorder comprises schizophrenia, autism spectrum disorder, attention-deficit/hyperactivity disorder, or a learning disorder. In some embodiments, the inflammatory disease comprises ulcerative colitis, Crohn's disease, rheumatoid arthritis, ankylosing spondylitis, insulin-dependent diabetes, Addison's disease, Goodpasture syndrome, IgA nephropathy, interstitial nephritis, Sjögren's syndrome, autoimmune pancreatitis, psoriasis, atopic dermatitis, pneumonia, chronic bronchitis, bronchial asthma, systemic lupus erythematosus (SLE), scleroderma, or delirium, and the bone disease comprises osteoporosis, osteolytic bone metastasis, or Paget's disease of bone.

The disclosure provides compositions for use in treating or ameliorating a depressive symptom in a subject, or treating a disease or disorder in a subject, the use comprising administering a therapeutically effective amount of a composition comprising R(−)-ketamine or a pharmaceutically acceptable salt thereof to the subject, wherein the composition is substantially free of S(+)-ketamine or a pharmaceutically acceptable salt thereof, and wherein the therapeutically effective amount comprises a maximum blood plasma concentration (Cmax) of R(−)-ketamine of at least 250 ng/mL.

The disclosure provides compositions for use in the manufacture of a medicament for treating or ameliorating a depressive symptom in a subject, or treating a disease or disorder in a subject, the use comprising administering a therapeutically effective amount of a composition comprising R(−)-ketamine or a pharmaceutically acceptable salt thereof to the subject, wherein the composition is substantially free of S(+)-ketamine or a pharmaceutically acceptable salt thereof, and wherein the therapeutically effective amount comprises a maximum blood plasma concentration (Cmax) of R(−)-ketamine of at least 250 ng/mL.

### BRIEF DESCRIPTION OF THE DRAWINGS

FIG. 1 is a plot showing the arithmetic mean (+/−SD, standard deviation) plasma-concentration time plot for R(−)-ketamine on a linear scale.

FIG. 2 is a plot showing the arithmetic mean (+/−SD) plasma-concentration time plot for R(−)-ketamine on a semi-logarithmic scale.

FIG. 3 is a plot showing the arithmetic mean (+/−SD, standard deviation) plasma-concentration time plot for norketamine on a linear scale.

FIG. 4 is a plot showing the arithmetic mean (+/−SD) plasma-concentration time plot for norketamine on a semi-logarithmic scale.

FIG. 5 is a plot showing the arithmetic mean (+/−SD, standard deviation) plasma-concentration time plot for 6-hydroxynorketamine on a linear scale.

FIG. 6 is a plot showing the arithmetic mean (+/−SD) plasma-concentration time plot for 6-hydroxynorketamine on a semi-logarithmic scale.

FIG. 7 is a plot showing the arithmetic mean (+/−SD, standard deviation) plasma-concentration time plot for dehydronorketamine on a linear scale.

FIG. 8 is a plot showing the arithmetic mean (+/−SD) plasma-concentration time plot for dehydronorketamine on a semi-logarithmic scale.

FIGS. 9A-9B show a table summarizing plasma parameters of R(−)-ketamine in the 6 cohorts administered R(−)-ketamine at the indicated doses. Abbreviations: CV %=Coefficient of variation; DN=Dose normalized; N=Number of subjects; SD=Standard deviation; PK=pharmacokinetic. Tₘₐₓ is represented with median (range). The clinical site confirmed that there is no documentation in the source to explain any irregularity with the sample collection process and the bioanalytical lab confirmed that the raw data is accurate.

FIGS. 10A-10B show a table summarizing plasma parameters of norketamine in the 6 cohorts administered R(−)-ketamine at the indicated doses. Abbreviations: CV %=Coefficient of variation; DN=Dose normalized; N=Number of subjects; SD=Standard deviation. Tₘₐₓ is represented with median (range).

FIGS. 11A-11B show a table summarizing plasma parameters of 6-hydroxynorketamine in the 6 cohorts administered R(−)-ketamine at the indicated doses. Abbreviations: CV %=Coefficient of variation; DN=Dose normalized; N=Number of subjects; SD=Standard deviation. Tₘₐₓ is represented with median (range).

FIGS. 12A-12B show a table summarizing plasma parameters of dehydronorketamine in the 6 cohorts administered R(−)-ketamine at the indicated doses. Abbreviations: CV %=Coefficient of variation; DN=Dose normalized; N=Number of subjects; SD=Standard deviation. Tₘₐₓ is represented with median (range).

FIG. 13 is a table showing a summary of dose proportionality for R(−)-ketamine and its metabolites. Abbreviations: N=Number of subjects; CI=Confidence interval.

FIG. 14 is a table showing a summary of plasma R(−) ketamine PK parameters after subcutaneous injection at the indicated dosages.

FIG. 15 is a table showing a summary of plasma R(−) ketamine PK parameters after intravenous injection at the indicated dosage.

### DETAILED DESCRIPTION

The World Health Organization reports that depression is the leading cause of disability and loss of work worldwide, affecting more than 300 million people (Non-Patent Literature 26). In severe cases, it can lead to suicide, which has become the third leading cause of death in the US with its incidence having increased 30% over the past 10 years (Non-Patent Literature 27, 28). The prevalence of treatment-resistant depression (TRD), which is generally defined as treatment failure with adequate trials of at least 2 antidepressant regimens, remains very high. A multicenter European study found that 51% of depressed patients recruited from specialist referral centers met such criteria (Non-Patent Literature 29). In addition, much of the cost and disability associated with depression is accounted for by TRD (Non-Patent Literature 30). Finally, it should be noted that many patients with major depressive disorder (MDD) continue to have residual symptoms even without meeting the formal diagnosis of TRD, thus adding to the unmet burden.

An existing treatment for TRD is the administration of a composition comprising ketamine, a compound that was approved for use as a parenterally administered anesthetic agent in the United States (US) in 1970. Since then, it has seen wide use in both adult and pediatric populations. Ketamine is available as two stereoisomers R(−)-ketamine and S(+)-ketamine, and the latter was approved for the treatment of TRD in adults in the US, and later approved in the European Union (EU) in 2019 as the isolated stereoisomer in an intranasal formulation (Spravato™). However, this product has been shown to have a limited therapeutic index, particularly with respect to dissociative side effects including illusions, distortions of time and space, derealization, and depersonalization. Further, in a human abuse potential study, the scores for “Drug Liking at the Moment” and “Take Drug Again” were similar to racemic ketamine, a known drug of abuse, and greater than placebo at both the maximum indicated antidepressant dose and 1.3 times this dose (Non-Patent Literature 1). Based in part upon these findings, the administration of Spravato is limited to the clinic and under the observation of medical personnel.

Both R(−)-ketamine and S(+)-ketamine (and by extension, racemic ketamine) share complex pharmacology with receptor-binding studies revealing significant affinity at several receptors and ion channels, e.g., glutamatergic, cholinergic, sigma, opioid, and hyperpolarization-activated cyclic nucleotide-gated channels. R(−)-ketamine and S(+)-ketamine are primarily regarded as N-methyl-D-aspartate (NMDA) receptor non-competitive antagonists, with S(+)-ketamine having approximately 4 times the binding affinity for the phencyclidine site of this receptor than R(−)-ketamine. Such pharmacological activity is thought to be the primary driver of the adverse psychotomimetic properties seen with ketamine, and S(+)-ketamine is suggested to mainly cause these effects. However, data suggest that such NMDA receptor activity is not the only driver of antidepressant efficacy. Indeed, nonclinical depression model studies of subanesthetic doses (10 mg/kg) in rodents suggest R(−)-ketamine possesses longer acting and more potent effects than S(+)-ketamine despite R(−)-ketamine's lower affinity to the NMDA receptor (Non-Patent Literature 2, 3, 5, 7, 9, 11, 12, 13, 14, 15, 16, 17, 18). Moreover, at doses that had effects in depression models in rodents (20 mg/kg), R(−)-ketamine did not cause conditioned place preference (Non-Patent Literature 2, 7), a nonclinical test that is thought to suggest a clinical risk of substance abuse. However, it should be noted that at a high dose (40 mg/kg), R(−)-ketamine did cause conditioned place preference in rodents (Non-Patent Literature 19). Nonclinical studies suggest that R(−)-ketamine could cause less dissociative and psychotomimetic effects at therapeutic doses and have a lower potential for misuse compared with S(+)-ketamine or racemic ketamine (Non-Patent Literature 2, 3, 4, 5, 6, 7, 8, 9, 10).

Both nonclinical and preliminary clinical studies suggest that R(−)-ketamine may have a more favorable safety profile with a decreased incidence of adverse events (AEs) (e.g., dissociative, cognitive impairment, and psychotomimetic effects) compared with S(+)-ketamine. Based on nonclinical studies, R(−)-ketamine may also have less abuse potential than S(+)-ketamine. Overall, the available data support the concept of the use of R(−)-ketamine as a potentially better tolerated, rapidly acting antidepressant compared with ketamine and S(+)-ketamine, which may support the use of R(−)-ketamine outside of a supervised, in-clinic environment. Although racemic ketamine has been extensively used for almost 50 years as an approved parenteral anesthetic worldwide, it has not been approved for the indication of TRD because of concerns over potential AEs related to repeated dosing, abuse liability, and its intravenous (IV) route of administration. There is thus a need for additional ketamine-like agents that reduce the side effects seen with ketamine and S(+)-ketamine while providing efficacy for depressive symptoms, and which have a convenient route of administration to permit the use outside of a supervised clinical setting.

### Definitions

The following explanations of terms and methods are provided to better describe the present disclosure and to guide those of ordinary skill in the art in the practice of the present disclosure.

The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

“Administering” refers to any suitable mode of administration, including, oral administration, administration as a suppository, topical contact, parenteral, intravenous, intraperitoneal, intramuscular, intralesional, intranasal administration, subcutaneous administration, intrathecal administration, or the implantation of a slow-release device e.g., a mini-osmotic pump, to the subject.

A “maximum blood plasma concentration” or “Cmax” refers to the highest concentration of a drug in the blood plasma after dose of the drug is given to a subject. Methods of measuring the concentration of drugs will be known to persons of ordinary skill in the art, and include, inter alia, liquid chromatography, and tandem mass spectrometry.

The “area under the curve” or “AUC” is the integral of the concentration of a drug in blood plasma as a function of time. The AUC can be determined for the totality of time for which data is available, for example until the drug is no longer detectable (AUC_{0-inf}) or for a particular truncated window of time, for example 24 hours after administration (AUC₀₋₂₄).

All publications, patents, and patent applications mentioned in this specification are herein incorporated by reference to the same extent as if each individual publication, patent, or patent application was specifically and individually indicated to be incorporated by reference. The contents of WO 2015/037248, published Mar. 19, 2015, WO 2019/213551, published Nov. 7, 2019, WO 2018/079693, published May 3, 2018, WO 2019/065900, published Apr. 4, 2019, WO 2019/160057, published Aug. 22, 2019, WO 2020/138491, published Jul. 2, 2020, WO 2023/064363, published Apr. 20, 2023, and WO 2023/178039, published Sep. 21, 2023, are incorporated by reference in their entireties herein.

### Methods of Treating Depressive Symptoms

The present disclosure relates to methods of treating or ameliorating depressive symptoms of a subject. The methods include administering a therapeutically effective amount of a composition comprising R(−)-ketamine, or a pharmaceutically acceptable salt thereof, to the subject.

The therapeutically effective amount can be an amount that, after administration, produces a maximum blood plasma concentration (Cmax) of R(−)-ketamine of at least 250 ng/ml in the subject. In some embodiments, the therapeutically effective amount of R(−)-ketamine comprises an amount of R(−)-ketamine that produces a Cmax of at least 100 ng/ml, at least 150 ng/mL, at least 200 ng/mL, at least 250 ng/mL, at least 300 ng/ml, at least 350 ng/mL, at least 400 ng/mL, at least 400 ng/mL, at least 500 ng/ml, at least 600 ng/ml, at least 700 ng/mL, at least 800 ng/ml, at least 900 ng/mL or at least 1000 ng/ml after administration to the subject. In some embodiments, the therapeutically effective amount of R(−)-ketamine comprises an amount of R(−)-ketamine that produces a Cmax of at least 200 ng/ml after administration to the subject. In some embodiments, the therapeutically effective amount of R(−)-ketamine comprises an amount of R(−)-ketamine that produces a Cmax of at least 300 ng/mL after administration to the subject. In some embodiments, the therapeutically effective amount of R(−)-ketamine comprises an amount of R(−)-ketamine that produces a Cmax of at least 400 ng/mL after administration to the subject. In some embodiments, the therapeutically effective amount of R(−)-ketamine comprises an amount of R(−)-ketamine that produces a Cmax of at least 500 ng/ml after administration to the subject. In some embodiments, the therapeutically effective amount of R(−)-ketamine comprises an amount of R(−)-ketamine that produces a Cmax of at least 600 ng/mL after administration to the subject. In some embodiments, the therapeutically effective amount of R(−)-ketamine comprises an amount of R(−)-ketamine that produces a Cmax of at least 700 ng/ml after administration to the subject. In some embodiments, the therapeutically effective amount of R(−)-ketamine comprises an amount of R(−)-ketamine that produces a Cmax of at least 800 ng/mL after administration to the subject. In some embodiments, the therapeutically effective amount of R(−)-ketamine comprises an amount of R(−)-ketamine that produces a Cmax of at least 900 ng/ml after administration to the subject.

In some embodiments, the therapeutically effective amount of R(−)-ketamine comprises an amount of R(−)-ketamine that produces a Cmax between 100 and 1000 ng/ml after administration to the subject. In some embodiments, the therapeutically effective amount of R(−)-ketamine comprises an amount of R(−)-ketamine that produces a Cmax between 200 and 900 ng/mL after administration to the subject. In some embodiments, the therapeutically effective amount of R(−)-ketamine comprises an amount of R(−)-ketamine that produces a Cmax between 250 and 900 ng/mL after administration to the subject. In some embodiments, the therapeutically effective amount of R(−)-ketamine comprises an amount of R(−)-ketamine that produces a Cmax between 2500 and 800 ng/mL after administration to the subject. In some embodiments, the therapeutically effective amount of R(−)-ketamine comprises an amount of R(−)-ketamine that produces a Cmax between 300 and 800 ng/ml after administration to the subject. In some embodiments, the therapeutically effective amount of R(−)-ketamine comprises an amount of R(−)-ketamine that produces a Cmax between 400 and 800 ng/mL after administration to the subject. In some embodiments, the therapeutically effective amount of R(−)-ketamine comprises an amount of R(−)-ketamine that produces a Cmax between 300 and 1000 ng/mL after administration to the subject. In some embodiments, the therapeutically effective amount of R(−)-ketamine comprises an amount of R(−)-ketamine that produces a Cmax between 300 and 900 ng/ml after administration to the subject.

The therapeutically effective amount can be an amount that, after administration, produces an AUC of between 400 and 3000. In some embodiments, the therapeutically effective amount of R(−)-ketamine comprises an amount of R-(−)-ketamine that produces an AUC of between about 1500 and about 4000, between about 1000 and about 3500, between about 500 and about 2500, between about 500 and about 2000, between about 500 and about 1900, between about 500 and about 1800, between about 600 and about 1850, or between about 600 and about 1750. In some embodiments, the therapeutically effective amount of R(−)-ketamine comprises an amount of R-(−)-ketamine that produces an AUC of between about 500 and about 2500. In some embodiments, the therapeutically effective amount of R(−)-ketamine comprises an amount of R-(−)-ketamine that produces an AUC of between about 500 and about 2000. In some embodiments, the therapeutically effective amount of R(−)-ketamine comprises an amount of R-(−)-ketamine that produces an AUC of between about 500 and about 1900. In some embodiments, the therapeutically effective amount of R(−)-ketamine comprises an amount of R-(−)-ketamine that produces an AUC of between about 600 and about 2000. In some embodiments, the AUC comprises an AUC_{0-inf}. In some embodiments, the AUC comprises a truncated AUC measured from 0 to 24 h after administration of the R-(−)-ketamine (AUC_{0-24 h}).

The depressive symptoms can be assessed by a Montgomery Åsberg Depression Rating Scale (MADRS) subject score. The MADRS is a ten-item diagnostic questionnaire that is administered by a clinician and measures the severity of depressive episodes in subjects with mood disorders. The ranges of the MADRS scores include: 0 to 6 (normal, i.e., free of symptoms), 7 to 19 (mild depression), 20 to 34 (moderate depression), and 34 or more (severe depression). In some embodiments, the MADRS subject score is decreased by administration of the composition.

In some embodiments, the MADRS subject score is decreased by about 2 to about 20, or about 3 to about 16, or about 4 to about 12, or about 5 to about 8 as a result of administration of the composition. In some embodiments, the MADRS subject score is decreased by about 2, 3, 4, 5, 6, 7, 8, 9, 10, 11, 12, 13, 14, 15, 16, 17, 18, 19, 20, 21, 22, 23, 24, or 25 as a result of administration of the composition.

In some embodiments, the MADRS subject score is decreased from about 12 hours to about 96 hours, or about 12 hours to about 72 hours, or about 12 hours to about 48 hours, or about 12 hours to about 24 hours after administration of the composition begins. In some embodiments, the MADRS subject score is decreased about 6, 12, 18, 24, 36, 48, 60, 72, 84, or 96 hours after administration of the composition begins. In some embodiments, the MADRS score is decreased by about 2 to about 20 when measured about 24 hours, about 7 days, about 10 days, about 14 days or about 21 days after administration of the composition begins.

In some embodiments, the MADRS subject score is decreased by at least 10%, at least 20%, at least 30%, at least 40%, at least 50%, at least 60%, at least 70%, or at least 80% when measured about 2 hours, about 4 hours, about 7 days, or about 14 days after administration of the composition begins. In some embodiments, the MADRS subject score is decreased by at least 10% when measured about 2 hours, about 4 hours, about 7 days, or about 14 days after administration of the composition begins. In some embodiments, the MADRS subject score is decreased by at least 20% when measured about 2 hours, about 4 hours, about 7 days, or about 14 days after administration of the composition begins. In some embodiments, the MADRS subject score is decreased by at least 30% when measured about 2 hours, about 4 hours, about 7 days, or about 14 days after administration of the composition begins. In some embodiments, the MADRS subject score is decreased by at least 40% when measured about 2 hours, about 4 hours, about 7 days, or about 14 days after administration of the composition begins. In some embodiments, the MADRS subject score is decreased by at least 50% when measured about 2 hours, about 4 hours, about 7 days, or about 14 days after administration of the composition begins. In some embodiments, the MADRS subject score is decreased by at least 60% when measured about 2 hours, about 4 hours, about 7 days, or about 14 days after administration of the composition begins. In some embodiments, the MADRS subject score is decreased by at least 70% when measured about 2 hours, about 4 hours, about 7 days, or about 14 days after administration of the composition begins. In some embodiments, the MADRS subject score is decreased by at least 80% when measured about 2 hours, about 4 hours, about 7 days, or about 14 days after administration of the composition begins. In some embodiments, the MADRS subject score is decreased by at least 90% when measured about 2 hours, about 4 hours, about 7 days, or about 14 days after administration of the composition begins.

In some embodiments, the MADRS subject score is less than, or equal to, 14, 15, 16, 17, 18, 19, 20, 21, or 22 when measured about 2 hours, about 4 hours, about 7 days, or about 14 days after administration of the composition begins. In some embodiments, the MADRS subject score is less than, or equal to, 18 when measured about 2 hours, about 4 hours, about 7 days, or about 14 days after administration of the composition begins. In some embodiments, the MADRS subject score is less than, or equal to, 17 when measured about 2 hours, about 4 hours, about 7 days, or about 14 days after administration of the composition begins. In some embodiments, the MADRS subject score is less than, or equal to, 16 when measured about 2 hours, about 4 hours, about 7 days, or about 14 days after administration of the composition begins. In some embodiments, the MADRS subject score is less than, or equal to, 14 when measured about 2 hours, about 4 hours, about 7 days, or about 14 days after administration of the composition begins. In some embodiments, the MADRS subject score is less than, or equal to, 12 when measured about 2 hours, about 4 hours, about 7 days, or about 14 days after administration of the composition begins. In some embodiments, the MADRS subject score is less than, or equal to, 10 when measured about 2 hours, about 4 hours, about 7 days, or about 14 days after administration of the composition begins. In some embodiments, the MADRS subject score is less than, or equal to, 8 when measured about 2 hours, about 4 hours, about 7 days, or about 14 days after administration of the composition begins. In some embodiments, the MADRS subject score is less than, or equal to, 6 when measured about 2 hours, about 4 hours, about 7 days, or about 14 days after administration of the composition begins.

R(−)-ketamine

In studies using a mouse model of depression R(−)-ketamine exhibited more potent antidepressant effects on the depression-like symptoms when compared to S(+)-ketamine, and the effects lasted for a longer period of time. In studies using a social defeat stress model in mice R(−)-ketamine showed more potent and long lasting anti-depressant effects when compared to S(+)-ketamine. Furthermore, administration of S(+)-ketamine induced side effects such as a hyperlocomotion, prepulse inhibition deficit, and drug dependence, while administration of R(−)-ketamine did not. Since R(−)-ketamine has a lower affinity for the NMDA receptor when compared to S(+)-ketamine, R(−)-ketamine is considered to have less psychotomimetic side effects and to produce negligible drug dependence. Therefore, R(−)-ketamine or a pharmaceutically acceptable salt thereof has rapid and long-lasting antidepressant effects and less side effects when compared to S(+)-ketamine.

In some embodiments, the composition comprising R(−)-ketamine or a pharmaceutically acceptable salt thereof is substantially free of S(+)-ketamine or a pharmaceutically acceptable salt thereof. R(−)-ketamine or a pharmaceutically acceptable salt thereof can be considered to be substantially free of S(+)-ketamine if the amount of S(+)-ketamine is such that the side effects associated with S(+)-ketamine are substantially reduced, or not present when the composition is administered to a subject. Exemplary side effects include, but are not limited to psychotomimetic effects, such as alteration of perception, mood, thought or mental state, for example anhedonia or negative affect. Additional side effects include, somnolence, dizziness, headache, dysarthria, paraesthesia, balance disorder, hypoaesthesia, lethargy, memory impairment, sedation, sensory disturbance, slow speech, derealization, confusional state, aversion, bruxism, dissociation, euphoria, logorrhoea and altered time perception.

In some embodiments, the compositions comprising R(−)-ketamine or a pharmaceutically acceptable salt thereof contains less than about 5% S(+)-ketamine or a pharmaceutically acceptable salt thereof, less than about 4% S(+)-ketamine or a pharmaceutically acceptable salt thereof, less than about 3% S(+)-ketamine or a pharmaceutically acceptable salt thereof, less than about 2% S(+)-ketamine or a pharmaceutically acceptable salt thereof, less than about 1% S(+)-ketamine or a pharmaceutically acceptable salt thereof, less than about 0.9% S(+)-ketamine or a pharmaceutically acceptable salt thereof, less than about 0.8% S(+)-ketamine or a pharmaceutically acceptable salt thereof, less than about 0.7% S(+)-ketamine or a pharmaceutically acceptable salt thereof, less than about 0.6% S(+)-ketamine or a pharmaceutically acceptable salt thereof, less than about 0.5% S(+)-ketamine or a pharmaceutically acceptable salt thereof, less than about 0.4% S(+)-ketamine or a pharmaceutically acceptable salt thereof, less than about 0.3% S(+)-ketamine or a pharmaceutically acceptable salt thereof, less than about 0.2% S(+)-ketamine or a pharmaceutically acceptable salt thereof, less than about 0.1% S(+)-ketamine or a pharmaceutically acceptable salt thereof, less than about 0.005% S(+)-ketamine or a pharmaceutically acceptable salt thereof or less than about 0.001% S(+)-ketamine or a pharmaceutically acceptable salt thereof.

The R(−)-ketamine may be a free base, a pharmaceutically acceptable salt thereof, or both. The pharmaceutically acceptable salt is preferably a pharmaceutically acceptable acid addition salt, more preferably a hydrochloride. The chemical structural formula of R(−)-ketamine hydrochloride is represented by the following formula (I).

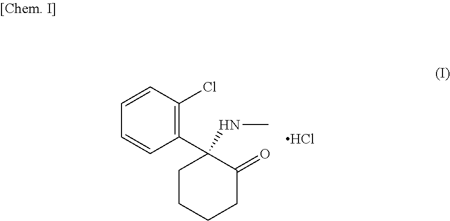 [SMILES: CN[C@@]1(c2ccccc2Cl)CCCCC1=O ; Cl]

R(−)-ketamine or a pharmaceutically acceptable salt thereof may be subjected to modification, for example, substitution of a chlorine molecule as a substituent by another halogen molecule and/or substitution of a methyl group as a substituent by another alkyl group, to thereby manufacture a derivative. Exemplary halogens include fluorine, chlorine, bromine, iodine, astatine and tennessine. As a result, a compound having more preferred effects may be obtained. Further, when the compound according to the present invention is labeled with an isotope such as a stable isotope ¹³C or ²H (D), the compound can be measured for its in vivo kinetics and quantitatively measured for its affinity for the NMDA receptor in the brain, for example.

In some embodiments, the composition comprises from about 5 to 1000 mg, or about 5 to 500 mg, or about 10 to 300 mg, or about 20 to 200 mg, or about 10 to 100 mg, or about 30 to 60 mg R(−)-ketamine or a pharmaceutically acceptable salt thereof. In some embodiments, the composition comprises about 30 mg R(−)-ketamine or a pharmaceutically acceptable salt thereof. In some embodiments, the composition comprises about 60 mg R(−)-ketamine or a pharmaceutically acceptable salt thereof.

In some embodiments, a therapeutically effective dose of the composition comprises between about 50 mg to 1000 mg, between about 50 mg to 500 mg, about 50 mg to 400 mg, about 50 mg to 300 mg, about 50 mg to 250 mg, about 50 mg to 200 mg, about 50 mg to 150 mg, about 50 mg to 100 mg, about 50 mg to 300 mg, about 50 mg to 250 mg, about 50 mg to 200 mg, about 50 mg to 150 mg, about 50 mg to 100 mg, about 100 mg to 300 mg, about 100 mg to 250 mg, about 100 mg to 200 mg, about 100 mg to 150 mg, about 150 mg to 300 mg, about 150 mg to 250 mg, or about 150 mg to 200 mg of R(−)-ketamine or a pharmaceutically acceptable salt thereof. In some embodiments, the therapeutically effective dose of the composition comprises about 50 mg to about 200 mg R(−)-ketamine or a pharmaceutically acceptable salt thereof. In some embodiments, the therapeutically effective dose of the composition comprises about 50 mg to about 150 mg R(−)-ketamine or a pharmaceutically acceptable salt thereof. In some embodiments, the therapeutically effective dose of the composition comprises about 100 mg to about 200 mg R(−)-ketamine or a pharmaceutically acceptable salt thereof.

In some embodiments, a therapeutically effective dose of the composition comprises 50 mg, 60 mg, 70 mg, 80 mg, 90 mg, 100 mg, 110 mg, 120 mg, 130 mg, 140 mg, 150 mg, 160 mg, 170 mg 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 420 mg, 440 mg, 450 mg, 460 mg, 480 mg, 500 mg, 520 mg, 550 mg, 580 mg or 600 mg of R(−)-ketamine or a pharmaceutically acceptable salt thereof. In some embodiments, a therapeutically effective dose of the composition comprises 60 mg of R(−)-ketamine or a pharmaceutically acceptable salt thereof. In some embodiments, a therapeutically effective dose of the composition comprises 80 mg of R(−)-ketamine or a pharmaceutically acceptable salt thereof. In some embodiments, a therapeutically effective dose of the composition comprises 90 mg of R(−)-ketamine or a pharmaceutically acceptable salt thereof. In some embodiments, a therapeutically effective dose of the composition comprises 100 mg of R(−)-ketamine or a pharmaceutically acceptable salt thereof. In some embodiments, a therapeutically effective dose of the composition comprises 120 mg of R(−)-ketamine or a pharmaceutically acceptable salt thereof. In some embodiments, a therapeutically effective dose of the composition comprises 150 mg of R(−)-ketamine or a pharmaceutically acceptable salt thereof. In some embodiments, a therapeutically effective dose of the composition comprises 200 mg of R(−)-ketamine or a pharmaceutically acceptable salt thereof. In some embodiments, a therapeutically effective dose of the composition comprises 250 mg of R(−)-ketamine or a pharmaceutically acceptable salt thereof. In some embodiments, a therapeutically effective dose of the composition comprises 300 mg of R(−)-ketamine or a pharmaceutically acceptable salt thereof. In some embodiments, a therapeutically effective dose of the composition comprises 350 mg of R(−)-ketamine or a pharmaceutically acceptable salt thereof. In some embodiments, a therapeutically effective dose of the composition comprises 400 mg of R(−)-ketamine or a pharmaceutically acceptable salt thereof. In some embodiments, a therapeutically effective dose of the composition comprises 450 mg of R(−)-ketamine or a pharmaceutically acceptable salt thereof. In some embodiments, a therapeutically effective dose of the composition comprises 500 mg of R(−)-ketamine or a pharmaceutically acceptable salt thereof.

In some embodiments, the composition comprises R(−)-ketamine hydrochloride. In some embodiments, the composition comprises from about 5 to 1000 mg, or about 5 to 500 mg, or about 10 to 300 mg, or about 20 to 200 mg, or about 10 to 100 mg, or about 30 to 60 mg R(−)-ketamine hydrochloride. In some embodiments, the composition comprises about 30 mg R(−)-ketamine hydrochloride. In some embodiments, the composition comprises about 60 mg R(−)-ketamine hydrochloride.

### Administration

The compositions of the present disclosure may be administered orally or parenterally. Examples of the parenteral administration include intravenous infusion, intramuscular infusion, subcutaneous infusion, intravenous injection, intramuscular injection, or subcutaneous injection. Transmucosal administration such as transnasal or oral administration using a spray, an aerosol, or the like; rectal administration using a suppository or the like; and transdermal or sublingual administration using a patch, a liniment, a gel, or the like. In the oral administration, a known dosage form for administration, including a tablet, a capsule, a coated tablet, a troche, or a liquid such as a solution or a suspension, may also be used. In some embodiments, the compositions may be administered intravenously or subcutaneously.

In some embodiments, the composition comprising R(−)-ketamine, or a pharmaceutically acceptable salt thereof, is administered by intravenous infusion, intravenous injection, subcutaneous infusion, or subcutaneous injection. In some embodiments, the composition of R(−)-ketamine is administered by intravenous infusion. In some embodiments, the composition of R(−)-ketamine is administered by subcutaneous injection.

In some embodiments, the composition of R(−)-ketamine hydrochloride is administered by intravenous infusion, intravenous injection, subcutaneous infusion, or subcutaneous injection. In some embodiments, the composition of R(−)-ketamine hydrochloride is administered by intravenous infusion. In some embodiments, the composition of R(−)-ketamine hydrochloride is administered by subcutaneous injection. In some embodiments, the composition of R(−)-ketamine hydrochloride in sterile water is administered by intravenous infusion, intravenous injection, subcutaneous infusion, or subcutaneous injection. In some embodiments, the composition of R(−)-ketamine hydrochloride in sterile water is administered by intravenous infusion. In some embodiments, the composition of R(−)-ketamine hydrochloride in sterile water is administered by subcutaneous injection.

The compositions of the present disclosure may be administered by intravenous infusion. In some embodiments, the compositions may be administered over a time period from about 10 minutes to about 3 hours. In some embodiments, the compositions may be administered over a time period of about 20 minutes, about 40 minutes, or about 1 hour. In some embodiments, the composition of R(−)-ketamine, or a pharmaceutically acceptable salt thereof, is administered over a time period from 5 minutes to 10 hours, or from 10 minutes to 3 hours, or from 20 minutes to 1 hour. In some embodiments, the composition of R(−)-ketamine hydrochloride is administered over a time period from 5 minutes to 10 hours, or from 10 minutes to 3 hours, or from 20 minutes to 1 hour. In some embodiments, the composition of R(−)-ketamine hydrochloride in sterile water is administered over a time period from 5 minutes to 10 hours, or from 10 minutes to 3 hours, or from 20 minutes to 1 hour. In some embodiments, the composition of R(−)-ketamine, or a pharmaceutically acceptable salt thereof, is administered over a time period of about 10 minutes, 20 minutes, 30 minutes, 40 minutes, 50 minutes, 1 hour, 2 hours or 3 hours. In some embodiments, the composition of R(−)-ketamine hydrochloride is administered over a time period of about 10 minutes, 20 minutes, 30 minutes, 40 minutes, 50 minutes, 1 hour, 2 hours or 3 hours. In some embodiments, the composition of R(−)-ketamine hydrochloride in sterile water is administered over a time period of about 10 minutes, 20 minutes, 30 minutes, 40 minutes, 50 minutes, 1 hour, 2 hours or 3 hours.

In some embodiments, the composition comprising R(−)-ketamine, or a pharmaceutically acceptable salt thereof, is administered via inhalation. For example, the composition comprising R(−)-ketamine, or a pharmaceutically acceptable salt thereof, may be administered intranasally in the form of an aerosol.

The compositions of the present disclosure may be administered once per day, one every other day, once every three days, once every four days, once every 5 days, once every 6 days, once per week, every other week, every 10 days, or once per month. In some embodiments, the composition of R(−)-ketamine, or a pharmaceutically acceptable salt thereof, is administered once per day. In some embodiments, the composition of R(−)-ketamine, or a pharmaceutically acceptable salt thereof, is administered twice per day. In some embodiments, the composition of R(−)-ketamine, or a pharmaceutically acceptable salt thereof, is administered 2-5 times per day. In some embodiments, the composition of R(−)-ketamine, or a pharmaceutically acceptable salt thereof, is administered every other day. In some embodiments, the composition of R(−)-ketamine, or a pharmaceutically acceptable salt thereof, is administered every three days. In some embodiments, the composition of R(−)-ketamine, or a pharmaceutically acceptable salt thereof, is administered every four days. In some embodiments, the composition of R(−)-ketamine, or a pharmaceutically acceptable salt thereof, is administered once per week. In some embodiments, the composition of R(−)-ketamine, or a pharmaceutically acceptable salt thereof, is administered twice per week. In some embodiments, the composition of R(−)-ketamine, or a pharmaceutically acceptable salt thereof, is administered every 10 days. In some embodiments, the composition of R(−)-ketamine, or a pharmaceutically acceptable salt thereof, is administered every other week. In some embodiments, the composition of R(−)-ketamine, or a pharmaceutically acceptable salt thereof, is administered 2-5 times per week. In some embodiments, the composition of R(−)-ketamine, or a pharmaceutically acceptable salt thereof, is administered once per month. In some embodiments, the composition of R(−)-ketamine, or a pharmaceutically acceptable salt thereof, is administered twice per month. In some embodiments, the composition of R(−)-ketamine, or a pharmaceutically acceptable salt thereof, is administered 2-5 times per month.

The compositions of the present disclosure can be administered according to a dosing schedule that includes an initial period in which the composition is administered at higher concentration, and/or with greater frequency, followed by a maintenance period in which the composition is administered at a lower dose and/or less frequently. In some embodiments, the dosing schedule comprises (a) an initial period in which the composition is administered every 1, 2, 3 or 4 days; and (b) a maintenance period in which composition is administered less frequently than during the initial period. In some embodiments, the composition is administered once per week, twice per week, every other week, every 10 days, or once per month during the maintenance period.

As used herein, a “therapeutically effective amount” refers to an amount of the R(−)-ketamine composition that is sufficient to treat or ameliorate the depressive symptoms of a subject as described herein. For example, a therapeutically effective amount of the R(−)-ketamine composition can decrease the MADRS subject score by about 2 to about 20, or about 3 to about 16, or about 4 to about 12, or about 5 to about 8, in a subject with depressive symptoms.

In some embodiments, the therapeutically effective amount of the R(−)-ketamine composition increases systolic blood pressure of the subject by less than 40 mmHg, less than 30 mmHg, less than 20 mmHg, or less than 10 mmHg. In some embodiments, the change in systolic blood pressure is measured within 1 hour, 5 hours, 1 day, 7 days or 14 days of administration.

In some embodiments, the therapeutically effective amount of the R(−)-ketamine composition increases systolic blood pressure of the subject by less than 40 mmHg, less than 30 mmHg, less than 20 mmHg, or less than 10 mmHg, as measured within 14 days of administration. In some embodiments, the therapeutically effective amount of the R(−)-ketamine composition increases systolic blood pressure of the subject by less than 40 mmHg as measured within 14 days of administration. In some embodiments, the therapeutically effective amount of the R(−)-ketamine composition increases systolic blood pressure of the subject by less than 30 mmHg as measured within 14 days of administration. In some embodiments, the therapeutically effective amount of the R(−)-ketamine composition increases systolic blood pressure of the subject by less than 20 mmHg as measured within 14 days of administration. In some embodiments, the therapeutically effective amount of the R(−)-ketamine composition increases systolic blood pressure of the subject by less than 10 mmHg as measured within 14 days of administration. In some embodiments, the therapeutically effective amount of the R(−)-ketamine composition increases systolic blood pressure of the subject by less than 3 mmHg as measured within 14 days of administration. In some embodiments, the therapeutically effective amount of the R(−)-ketamine composition increases systolic blood pressure of the subject by less than 40 mmHg as measured within 14 days of administration.

In some embodiments, the therapeutically effective amount of the R(−)-ketamine composition increases diastolic blood pressure of the subject by less than 35 mmHg, less than 30 mmHg, less than 25 mmHg, less than 20 mmHg, less than 15 mmHg, less than 10 mmHg, or less than 50 mmHg. In some embodiments, the change in diastolic blood pressure is measured within 1 hour, 5 hours, 1 day, 7 days or 14 days of administration.

In some embodiments, the therapeutically effective amount of the R(−)-ketamine composition increases diastolic blood pressure of the subject by less than 40 mmHg, less than 30 mmHg, less than 20 mmHg, or less than 10 mmHg, as measured within 14 days of administration. In some embodiments, the therapeutically effective amount of the R(−)-ketamine composition increases diastolic blood pressure of the subject by less than 40 mmHg as measured within 14 days of administration. In some embodiments, the therapeutically effective amount of the R(−)-ketamine composition increases diastolic blood pressure of the subject by less than 30 mmHg as measured within 14 days of administration. In some embodiments, the therapeutically effective amount of the R(−)-ketamine composition increases diastolic blood pressure of the subject by less than 20 mmHg as measured within 14 days of administration. In some embodiments, the therapeutically effective amount of the R(−)-ketamine composition increases diastolic blood pressure of the subject by less than 10 mmHg as measured within 14 days of administration. In some embodiments, the therapeutically effective amount of the R(−)-ketamine composition increases diastolic blood pressure of the subject by less than 3 mmHg as measured within 14 days of administration. In some embodiments, the therapeutically effective amount of the R(−)-ketamine composition increases diastolic blood pressure of the subject by less than 40 mmHg as measured within 14 days of administration.

In some embodiments, administration of the therapeutically effective amount of the R(−)-ketamine composition has fewer side effects or adverse events than administration of a therapeutically effective amount of S(+)-ketamine or racemic ketamine. In some embodiments, administration of the therapeutically effective amount of the R(−)-ketamine composition has fewer side effects or adverse events than administration of a similar amount of S(+)-ketamine or racemic ketamine.

### Pharmaceutical Formulations

The present disclosure also relates to pharmaceutical formulations that comprise a pharmaceutically acceptable carrier and R(−)-ketamine, or a pharmaceutically acceptable salt thereof, or the composition comprising R(−)-ketamine, or a pharmaceutically acceptable salt thereof, as disclosed herein. In some embodiments, the pharmaceutical formulations comprise a pharmaceutically acceptable carrier. In some embodiments, the pharmaceutical formulations comprise a pharmaceutically acceptable carrier and R(−)-ketamine hydrochloride. Examples of the pharmaceutically acceptable carrier include an antioxidant, a stabilizer, a preservative, a taste-masking agent, a colorant, a solubilizer, a solubilizing agent, a surfactant, an emulsifier, an antifoaming agent, a viscosity adjustor, a gelling agent, an absorption accelerator, a dispersant, an excipient, and a pH adjustor.

In some embodiments, pharmaceutical formulations of the disclosure comprise a pH adjustor or buffering agent. Suitable buffers will be known to persons of ordinary skill in the art, and include, inter alia, a succinate buffer, tartrate buffer, maleate buffer, a fumarate buffer, a citrate buffer and an acetate buffer.

In some embodiments, the pharmaceutical formulations may be prepared as formulations for administration. In some embodiments, the pharmaceutical formulations for administration by infusion or injection may be prepared in the form of a solution or a suspension. In some embodiments, the pharmaceutical formulations for transmucosal administration such as transnasal or oral administration, may be prepared in the form of a powder, a drop, or an aerosol. In some embodiments, the pharmaceutical formulations for rectal administration may be prepared in the form of a semi-solid formulation such as a cream or a suppository. In some embodiments, the pharmaceutical formulations for sublingual administration may be prepared in the form of a fast dissolving strip or tablet. Each of the formulations may be prepared by any one of the methods known to those skilled in the art of pharmacy as disclosed in, for example, Remington's Pharmaceutical Sciences (Mack Publishing Company, Easton, PA, 1970).

The accurate dosage and dosing regimen of the compositions and pharmaceutical formulations disclosed herein may be adjusted depending on required amounts, treatment methods, diseases, degrees of necessity, or the like for individual treatment targets. The dosage may be specifically determined depending on an age, a body weight, a general health condition, a sex, a meal, an administration time, an administration method, an elimination rate, a combination of drugs, a medical condition of a patient, and the like, and may be determined in consideration of other factors. When the compositions or pharmaceutical formulations are administered to individuals with substance use disorders exhibiting symptoms such as anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing or substance craving, it is preferred that an active ingredient contained in the composition or pharmaceutical formulation be limited to an effective amount for reducing symptoms of the substance use disorder. R(−)-ketamine or a pharmaceutically acceptable salt thereof can be safely used because of having less side effects found in S(+)-ketamine and racemic ketamine. Its dosage per day varies depending on the condition and body weight of a patient, the kind of a compound, an administration route, and the like.

### Subcutaneous Formulation and Administration

In some embodiments, the formulations can include prefilled syringes, injectors, vials, powder for infusion for reconstitution, concentrate for infusion to be diluted before delivery (ready to dilute) or solutions (ready to use).

In some embodiments, any of the salts or salt forms described in International Application Publication No. WO 2023/064363 are contemplated in each aspect and/or embodiment as described herein. International Application Publication No. WO 2023/064363 is hereby incorporated by reference in its entirety.

In some embodiments, formulations can be aqueous isotonic solutions or suspensions.

For subcutaneous use, a sterile solution may be employed. In some embodiments, the total concentration of the solute(s) is controlled to render the formulation isotonic.

In some embodiments, the formulation for subcutaneous injection comprising R(−)-ketamine comprises R(−)-ketamine at a concentration between about 60 mg/mL and about 150 mg/mL.

In some embodiments, the formulation for subcutaneous injection comprising R(−)-ketamine comprises R(−)-ketamine at a concentration between about 70 mg/mL and about 150 mg/mL. In some embodiments, the formulation for subcutaneous injection comprising R(−)-ketamine comprises R(−)-ketamine at a concentration between about 80 mg/mL and about 150 mg/mL. In some embodiments, the formulation for subcutaneous injection comprising R(−)-ketamine comprises R(−)-ketamine at a concentration between about 90 mg/mL and about 150 mg/mL. In some embodiments, the formulation for subcutaneous injection comprising R(−)-ketamine comprises R(−)-ketamine at a concentration between about 100 mg/mL and about 150 mg/mL. In some embodiments, the formulation for subcutaneous injection comprising R(−)-ketamine comprises R(−)-ketamine at a concentration between about 110 mg/mL and about 150 mg/mL. In some embodiments, the formulation for subcutaneous injection comprising R(−)-ketamine comprises R(−)-ketamine at a concentration between about 120 mg/mL and about 150 mg/mL. In some embodiments, the formulation for subcutaneous injection comprising R(−)-ketamine comprises R(−)-ketamine at a concentration between about 130 mg/mL and about 150 mg/mL. In some embodiments, the formulation for subcutaneous injection comprising R(−)-ketamine comprises R(−)-ketamine at a concentration between about 140 mg/mL and about 150 mg/mL. In some embodiments, the formulation for subcutaneous injection comprising R(−)-ketamine comprises R(−)-ketamine at a concentration between about 60 mg/mL and about 140 mg/mL. In some embodiments, the formulation for subcutaneous injection comprising R(−)-ketamine comprises R(−)-ketamine at a concentration between about 70 mg/mL and about 140 mg/mL. In some embodiments, the formulation for subcutaneous injection comprising R(−)-ketamine comprises R(−)-ketamine at a concentration between about 80 mg/mL and about 140 mg/mL. In some embodiments, the formulation for subcutaneous injection comprising R(−)-ketamine comprises R(−)-ketamine at a concentration between about 90 mg/mL and about 140 mg/mL. In some embodiments, the formulation for subcutaneous injection comprising R(−)-ketamine comprises R(−)-ketamine at a concentration between about 100 mg/mL and about 140 mg/mL. In some embodiments, the formulation for subcutaneous injection comprising R(−)-ketamine comprises R(−)-ketamine at a concentration between about 110 mg/mL and about 140 mg/mL. In some embodiments, the formulation for subcutaneous injection comprising R(−)-ketamine comprises R(−)-ketamine at a concentration between about 120 mg/mL and about 140 mg/mL. In some embodiments, the formulation for subcutaneous injection comprising R(−)-ketamine comprises R(−)-ketamine at a concentration between about 130 mg/mL and about 140 mg/mL. In some embodiments, the formulation for subcutaneous injection comprising R(−)-ketamine comprises R(−)-ketamine at a concentration between about 60 mg/mL and about 130 mg/mL. In some embodiments, the formulation for subcutaneous injection comprising R(−)-ketamine comprises R(−)-ketamine at a concentration between about 70 mg/mL and about 130 mg/mL. In some embodiments, the formulation for subcutaneous injection comprising R(−)-ketamine comprises R(−)-ketamine at a concentration between about 80 mg/mL and about 130 mg/mL. In some embodiments, the formulation for subcutaneous injection comprising R(−)-ketamine comprises R(−)-ketamine at a concentration between about 90 mg/mL and about 130 mg/mL. In some embodiments, the formulation for subcutaneous injection comprising R(−)-ketamine comprises R(−)-ketamine at a concentration between about 100 mg/mL and about 130 mg/mL. In some embodiments, the formulation for subcutaneous injection comprising R(−)-ketamine comprises R(−)-ketamine at a concentration between about 110 mg/mL and about 130 mg/mL. In some embodiments, the formulation for subcutaneous injection comprising R(−)-ketamine comprises R(−)-ketamine at a concentration between about 120 mg/mL and about 130 mg/mL. In some embodiments, the formulation for subcutaneous injection comprising R(−)-ketamine comprises R(−)-ketamine at a concentration between about 60 mg/mL and about 120 mg/mL. In some embodiments, the formulation for subcutaneous injection comprising R(−)-ketamine comprises R(−)-ketamine at a concentration between about 70 mg/mL and about 120 mg/mL. In some embodiments, the formulation for subcutaneous injection comprising R(−)-ketamine comprises R(−)-ketamine at a concentration between about 80 mg/mL and about 120 mg/mL. In some embodiments, the formulation for subcutaneous injection comprising R(−)-ketamine comprises R(−)-ketamine at a concentration between about 90 mg/mL and about 120 mg/mL. In some embodiments, the formulation for subcutaneous injection comprising R(−)-ketamine comprises R(−)-ketamine at a concentration between about 100 mg/mL and about 120 mg/mL. In some embodiments, the formulation for subcutaneous injection comprising R(−)-ketamine comprises R(−)-ketamine at a concentration between about 110 mg/mL and about 120 mg/mL. In some embodiments, the formulation for subcutaneous injection comprising R(−)-ketamine comprises R(−)-ketamine at a concentration between about 60 mg/mL and about 110 mg/mL. In some embodiments, the formulation for subcutaneous injection comprising R(−)-ketamine comprises R(−)-ketamine at a concentration between about 70 mg/mL and about 110 mg/mL. In some embodiments, the formulation for subcutaneous injection comprising R(−)-ketamine comprises R(−)-ketamine at a concentration between about 80 mg/mL and about 110 mg/mL. In some embodiments, the formulation for subcutaneous injection comprising R(−)-ketamine comprises R(−)-ketamine at a concentration between about 90 mg/mL and about 110 mg/mL. In some embodiments, the formulation for subcutaneous injection comprising R(−)-ketamine comprises R(−)-ketamine at a concentration between about 100 mg/mL and about 110 mg/mL. In some embodiments, the formulation for subcutaneous injection comprising R(−)-ketamine comprises R(−)-ketamine at a concentration between about 60 mg/mL and about 100 mg/mL. In some embodiments, the formulation for subcutaneous injection comprising R(−)-ketamine comprises R(−)-ketamine at a concentration between about 70 mg/mL and about 100 mg/mL. In some embodiments, the formulation for subcutaneous injection comprising R(−)-ketamine comprises R(−)-ketamine at a concentration between about 80 mg/mL and about 100 mg/mL. In some embodiments, the formulation for subcutaneous injection comprising R(−)-ketamine comprises R(−)-ketamine at a concentration between about 90 mg/mL and about 100 mg/mL. In some embodiments, the formulation for subcutaneous injection comprising R(−)-ketamine comprises R(−)-ketamine at a concentration between about 60 mg/mL and about 90 mg/mL. In some embodiments, the formulation for subcutaneous injection comprising R(−)-ketamine comprises R(−)-ketamine at a concentration between about 70 mg/mL and about 90 mg/mL. In some embodiments, the formulation for subcutaneous injection comprising R(−)-ketamine comprises R(−)-ketamine at a concentration between about 80 mg/mL and about 90 mg/mL. In some embodiments, the formulation for subcutaneous injection comprising R(−)-ketamine comprises R(−)-ketamine at a concentration between about 60 mg/mL and about 80 mg/mL. In some embodiments, the formulation for subcutaneous injection comprising R(−)-ketamine comprises R(−)-ketamine at a concentration between about 70 mg/mL and about 80 mg/mL. In some embodiments, the formulation for subcutaneous injection comprising R(−)-ketamine comprises R(−)-ketamine at a concentration between about 60 mg/mL and about 70 mg/mL. In some embodiments, the formulation for subcutaneous injection comprising R(−)-ketamine comprises R(−)-ketamine at a concentration between about 95 mg/mL and about 110 mg/mL. In some embodiments, the formulation for subcutaneous injection comprising R(−)-ketamine comprises R(−)-ketamine at a concentration between about 90 mg/mL and about 105 mg/mL. In some embodiments, the formulation for subcutaneous injection comprising R(−)-ketamine comprises R(−)-ketamine at a concentration between about 95 mg/mL and about 105 mg/mL. In some embodiments, the formulation for subcutaneous injection comprising R(−)-ketamine comprises R(−)-ketamine at a concentration of about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL or about 110 mg/mL.

In some embodiments, the formulation for subcutaneous injection comprising R(−)-ketamine comprises R(−)-ketamine at a concentration of about 60 mg/mL. In some embodiments, the formulation for subcutaneous injection comprising R(−)-ketamine comprises R(−)-ketamine at a concentration of about 65 mg/mL. In some embodiments, the formulation for subcutaneous injection comprising R(−)-ketamine comprises R(−)-ketamine at a concentration of about 70 mg/mL. In some embodiments, the formulation for subcutaneous injection comprising R(−)-ketamine comprises R(−)-ketamine at a concentration of about 75 mg/mL. In some embodiments, the formulation for subcutaneous injection comprising R(−)-ketamine comprises R(−)-ketamine at a concentration of about 80 mg/mL. In some embodiments, the formulation for subcutaneous injection comprising R(−)-ketamine comprises R(−)-ketamine at a concentration of about 85 mg/mL. In some embodiments, the formulation for subcutaneous injection comprising R(−)-ketamine comprises R(−)-ketamine at a concentration of about 90 mg/mL. In some embodiments, the formulation for subcutaneous injection comprising R(−)-ketamine comprises R(−)-ketamine at a concentration of about 95 mg/mL. In some embodiments, the formulation for subcutaneous injection comprising R(−)-ketamine comprises R(−)-ketamine at a concentration of about 100 mg/mL. In some embodiments, the formulation for subcutaneous injection comprising R(−)-ketamine comprises R(−)-ketamine at a concentration of about 105 mg/mL. In some embodiments, the formulation for subcutaneous injection comprising R(−)-ketamine comprises R(−)-ketamine at a concentration of about 110 mg/mL.

In some embodiments, the formulation comprises substantially pure R(−)-ketamine. In some embodiments, the formulation is substantially free of S-ketamine. In some embodiments, over 90%, over 95%, over 96%, over 97%, over 98%, or over 99% of the ketamine is R(−)-ketamine. Alternatively, or in addition, R(−)-ketamine that is substantially free of S-ketamine refers to R(−)-ketamine in which S-ketamine is below the limit of detection using conventional methods in the art. As a further alternative R(−)-ketamine that is substantially free of S-ketamine refers to R(−)-ketamine in which the amount of S-ketamine is such that the side effects associated with S(+)-ketamine are substantially reduced, or not present when the composition is administered to a subject. Exemplary side effects include, but are not limited to psychotomimetic effects, such as alteration of perception, mood, thought or mental state, for example anhedonia or negative affect. Additional side effects include, somnolence, dizziness, headache, dysarthria, paraesthesia, balance disorder, hypoaesthesia, lethargy, memory impairment, sedation, sensory disturbance, slow speech, derealization, confusional state, aversion, bruxism, dissociation, euphoria, logorrhoea and altered time perception.

In some embodiments, over 90% of the ketamine is R(−)-ketamine. In some embodiments, over 95% of the ketamine is R(−)-ketamine. In some embodiments, over 96% of the ketamine is R(−)-ketamine. In some embodiments, over 97% of the ketamine is R(−)-ketamine. In some embodiments, over 98% of the ketamine is R(−)-ketamine. In some embodiments, over 99% of the ketamine is R(−)-ketamine. In some embodiments, over 99.5% of the ketamine is R(−)-ketamine. In some embodiments, over 99.9% of the ketamine is R(−)-ketamine.

In some embodiments, the formulation is a liquid formulation for subcutaneous administration comprising R(−)-ketamine. In some embodiments, the formulation is a liquid formulation for subcutaneous administration comprising non-ionized R(−)-ketamine (i.e., free base). In some embodiments, the formulation is a liquid formulation for subcutaneous administration comprising R(−)-ketamine in a mixture of ionized and non-ionized (free base) forms. In some embodiments, the formulation is a liquid formulation for subcutaneous administration comprising R(−)-ketamine in an ionized form. In some embodiments, the formulation is a liquid formulation for subcutaneous administration comprising R(−)-ketamine in a salt form.

In some embodiments, the pH of the injectable formulation of the present disclosure may be adjusted to a suitable pH. In some embodiments, the formulation pH is adjusted using a weak base. In some embodiments, the formulation pH is adjusted using a dilute base. In some embodiments, the formulation pH is adjusted using a strong base. In some embodiments, the formulation pH is adjusted using a weak and dilute base. In some embodiments, the formulation pH is adjusted using a strong and dilute base. In some embodiments, the formulation pH is adjusted using NaOH. In some embodiments, the formulation pH is adjusted using KOH. In some embodiments, the formulation pH is adjusted using IN NaOH. In some embodiments, the formulation pH is adjusted using IN KOH. In some embodiments, the formulation pH is adjusted using 0.5N NaOH. In some embodiments, the formulation pH is adjusted using 0.5N KOH.

In some embodiments, the formulation pH is adjusted using a weak acid. In some embodiments, the formulation pH is adjusted using a dilute acid. In some embodiments, the formulation pH is adjusted using a strong acid. In some embodiments, the formulation pH is adjusted using a weak and dilute acid. In some embodiments, the formulation pH is adjusted using a strong and dilute acid.

In some embodiments, the method further comprises adjusting the pH of the pharmaceutical composition. In some embodiments, only a minimal adjustment of the pH is necessary. In some embodiments, the pH is adjusted with a strong base. In some embodiments, the pH is adjusted with sodium hydroxide, potassium hydroxide, barium hydroxide, cesium hydroxide, strontium hydroxide magnesium hydroxide, calcium hydroxide, lithium hydroxide, or rubidium hydroxide.

In some embodiments, the pH is adjusted with sodium hydroxide. In some embodiments, the pH is adjusted to a desired pH. In some embodiments, the formulation is adjusted to a pH of about 5.0. In some embodiments, the formulation is adjusted to a pH of about 5.1. In some embodiments, the formulation is adjusted to a pH of about 5.2. In some embodiments, the formulation is adjusted to a pH of about 5.3. In some embodiments, the formulation is adjusted to a pH of about 5.4. In some embodiments, the formulation is adjusted to a pH of about 5.5. In some embodiments, the formulation is adjusted to a pH of about 5.6. In some embodiments, the formulation is adjusted to a pH of about 5.70. In some embodiments, the formulation is adjusted to a pH of about 5.71. In some embodiments, the formulation is adjusted to a pH of about 5.72. In some embodiments, the formulation is adjusted to a pH of about 5.73. In some embodiments, the formulation is adjusted to a pH of about 5.74. In some embodiments, the formulation is adjusted to a pH of about 5.75. In some embodiments, the formulation is adjusted to a pH of about 5.76. In some embodiments, the formulation is adjusted to a pH of about 5.77. In some embodiments, the formulation is adjusted to a pH of about 5.78. In some embodiments, the formulation is adjusted to a pH of about 5.79. In some embodiments, the formulation is adjusted to a pH of about 5.8. In some embodiments, the formulation is adjusted to a pH of about 5.9. In some embodiments, the formulation is adjusted to a pH of about 6.0. In some embodiments, the formulation is adjusted to a pH of between 5.0 and 6.0.

In some embodiments, the pH of the formulation is between about 5.0 and about 6.0. In some embodiments, the pH of the formulation is between about 5.1 and about 6.0. In some embodiments, the pH of the formulation is between about 5.2 and about 6.0. In some embodiments, the pH of the formulation is between about 5.3 and about 6.0. In some embodiments, the pH of the formulation is between about 5.4 and about 6.0. In some embodiments, the pH of the formulation is between about 5.5 and about 6.0. In some embodiments, the pH of the formulation is between about 5.6 and about 6.0. In some embodiments, the pH of the formulation is between about 5.7 and about 6.0. In some embodiments, the pH of the formulation is between about 5.8 and about 6.0. In some embodiments, the pH of the formulation is between about 5.9 and about 6.0.

In some embodiments, the pH of the formulation is between about 5.0 and about 5.9. In some embodiments, the pH of the formulation is between about 5.1 and about 5.9. In some embodiments, the pH of the formulation is between about 5.2 and about 5.9. In some embodiments, the pH of the formulation is between about 5.3 and about 5.9. In some embodiments, the pH of the formulation is between about 5.4 and about 5.9. In some embodiments, the pH of the formulation is between about 5.5 and about 5.9. In some embodiments, the pH of the formulation is between about 5.6 and about 5.9. In some embodiments, the pH of the formulation is between about 5.7 and about 5.9. In some embodiments, the pH of the formulation is between about 5.8 and about 5.9.

In some embodiments, the pH of the formulation is between about 5.0 and about 5.8. In some embodiments, the pH of the formulation is between about 5.1 and about 5.8. In some embodiments, the pH of the formulation is between about 5.2 and about 5.8. In some embodiments, the pH of the formulation is between about 5.3 and about 5.8. In some embodiments, the pH of the formulation is between about 5.4 and about 5.8. In some embodiments, the pH of the formulation is between about 5.5 and about 5.8. In some embodiments, the pH of the formulation is between about 5.6 and about 5.8. In some embodiments, the pH of the formulation is between about 5.7 and about 5.8.

In some embodiments, the pH of the formulation is between about 5.0 and about 5.7. In some embodiments, the pH of the formulation is between about 5.1 and about 5.7. In some embodiments, the pH of the formulation is between about 5.2 and about 5.7. In some embodiments, the pH of the formulation is between about 5.3 and about 5.7. In some embodiments, the pH of the formulation is between about 5.4 and about 5.7. In some embodiments, the pH of the formulation is between about 5.5 and about 5.7. In some embodiments, the pH of the formulation is between about 5.6 and about 5.7.

In some embodiments, the pH of the formulation is between about 5.0 and about 5.6. In some embodiments, the pH of the formulation is between about 5.1 and about 5.6. In some embodiments, the pH of the formulation is between about 5.2 and about 5.6. In some embodiments, the pH of the formulation is between about 5.3 and about 5.6. In some embodiments, the pH of the formulation is between about 5.4 and about 5.6. In some embodiments, the pH of the formulation is between about 5.5 and about 5.6.

In some embodiments, the pH of the formulation is between about 5.0 and about 5.5. In some embodiments, the pH of the formulation is between about 5.1 and about 5.5. In some embodiments, the pH of the formulation is between about 5.2 and about 5.5. In some embodiments, the pH of the formulation is between about 5.3 and about 5.5. In some embodiments, the pH of the formulation is between about 5.4 and about 5.5.

In some embodiments, the pH of the formulation is between about 5.0 and about 5.4. In some embodiments, the pH of the formulation is between about 5.1 and about 5.4. In some embodiments, the pH of the formulation is between about 5.2 and about 5.4. In some embodiments, the pH of the formulation is between about 5.3 and about 5.4.

In some embodiments, the pH of the formulation is between about 5.0 and about 5.3. In some embodiments, the pH of the formulation is between about 5.1 and about 5.3. In some embodiments, the pH of the formulation is between about 5.2 and about 5.3.

In some embodiments, the pH of the formulation is between about 5.0 and about 5.2. In some embodiments, the pH of the formulation is between about 5.1 and about 5.2.

In some embodiments, the pH of the formulation is between about 5.0 and about 5.1.

In some embodiments, the pH of the formulation is about 5.0. In some embodiments, the pH of the formulation is about 5.1. In some embodiments, the pH of the formulation is about 5.2. In some embodiments, the pH of the formulation is about 5.3. In some embodiments, the pH of the formulation is about 5.4. In some embodiments, the pH of the formulation is about 5.5. In some embodiments, the pH of the formulation is about 5.6. In some embodiments, the pH of the formulation is about 5.70. In some embodiments, the pH of the formulation is about 5.71. In some embodiments, the pH of the formulation is about 5.72. In some embodiments, the pH of the formulation is about 5.73. In some embodiments, the pH of the formulation is about 5.74. In some embodiments, the pH of the formulation is about 5.75. In some embodiments, the pH of the formulation is about 5.76. In some embodiments, the pH of the formulation is about 5.77. In some embodiments, the pH of the formulation is about 5.78. In some embodiments, the pH of the formulation is about 5.79. In some embodiments, the pH of the formulation is about 5.8. In some embodiments, the pH of the formulation is about 5.9. In some embodiments, the pH of the formulation is about 6.0.

In some embodiments, the R(−)-ketamine formulation does not comprise a cyclodextrin. In some embodiments, the R(−)-ketamine formulation does not comprise a cyclodextrin or a cyclodextrin derivative.

In those embodiments where the composition is administered by injection, the injectable composition may be supplied in various delivery forms, e.g. ampoules, pre-filled syringes, needle or needle free auto-injectors, as a small volume infusion or in multi-dose containers with an added preservative.

In some embodiments of the present disclosure, the parenteral composition is formulated as a subcutaneous injection.

Each of the injection formulations of the disclosure (e.g. a subcutaneous injection formulation) also include water, which may be present as a saline solution, to further dilute the formulation to an appropriate volume suitable for injection.

A suitable volume of a R(−)-ketamine formulation of the disclosure for use in an injection device (e.g. for subcutaneous administration) is in the range of about 0.1 mL to about 10 mL. Suitable volumes may include, for example, 0.1 mL, 0.2 mL, 0.3 mL, 0.4 mL, 0.5 mL, 0.6 mL, 0.7 mL, 0.8 mL, 0.9 mL, 1.0 mL, 2.0, mL, 3.0 mL, 4.0 mL, 5.0 mL, 6.0 mL, 7.0 mL, 8.0 mL, 9.0 mL, 10.0 mL and all amounts therebetween.

In some embodiments, the formulation has an osmolality of from about 150 mOsm/kg to about 850 mOsm/kg. In some embodiments, the formulation has an osmolality of from about 175 mOsm/kg to about 850 mOsm/kg. In some embodiments, the formulation has an osmolality of from about 200 mOsm/kg to about 850 mOsm/kg. In some embodiments, the formulation has an osmolality of from about 225 mOsm/kg to about 850 mOsm/kg. In some embodiments, the formulation has an osmolality of from about 250 mOsm/kg to about 850 mOsm/kg. In some embodiments, the formulation has an osmolality of from about 275 mOsm/kg to about 850 mOsm/kg. In some embodiments, the formulation has an osmolality of from about 300 mOsm/kg to about 850 mOsm/kg. In some embodiments, the formulation has an osmolality of from about 325 mOsm/kg to about 850 mOsm/kg. In some embodiments, the formulation has an osmolality of from about 350 mOsm/kg to about 850 mOsm/kg. In some embodiments, the formulation has an osmolality of from about 375 mOsm/kg to about 850 mOsm/kg. In some embodiments, the formulation has an osmolality of from about 400 mOsm/kg to about 850 mOsm/kg. In some embodiments, the formulation has an osmolality of from about 300 mOsm/kg to about 450 mOsm/kg. In some embodiments, the formulation has an osmolality of from about 475 mOsm/kg to about 850 mOsm/kg. In some embodiments, the formulation has an osmolality of from about 500 mOsm/kg to about 850 mOsm/kg.

In some embodiments, the formulation has an osmolality of at least about 150 mOsm/kg. In some embodiments, the formulation has an osmolality of at least about 175 mOsm/kg. In some embodiments, the formulation has an osmolality of at least about 200 mOsm/kg. In some embodiments, the formulation has an osmolality of at least about 225 mOsm/kg. In some embodiments, the formulation has an osmolality of at least about 250 mOsm/kg. In some embodiments, the formulation has an osmolality of at least about 275 mOsm/kg. In some embodiments, the formulation has an osmolality of at least about 300 mOsm/kg. In some embodiments, the formulation has an osmolality of at least about 325 mOsm/kg. In some embodiments, the formulation has an osmolality of at least about 350 mOsm/kg. In some embodiments, the formulation has an osmolality of at least about 375 mOsm/kg. In some embodiments, the formulation has an osmolality of at least about 400 mOsm/kg. In some embodiments, the formulation has an osmolality of at least about 425 mOsm/kg. In some embodiments, the formulation has an osmolality of at least about 450 mOsm/kg. In some embodiments, the formulation has an osmolality of at least about 475 mOsm/kg. In some embodiments, the formulation has an osmolality of at least about 500 mOsm/kg.

In some embodiments, the formulation has an osmolality of about <850 mOsm/kg. In some embodiments, the formulation has an osmolality of about <825 mOsm/kg. In some embodiments, the formulation has an osmolality of about <800 mOsm/kg. In some embodiments, the formulation has an osmolality of about <775 mOsm/kg. In some embodiments, the formulation has an osmolality of about <750 mOsm/kg. In some embodiments, the formulation has an osmolality of about <725 mOsm/kg. In some embodiments, the formulation has an osmolality of about <700 mOsm/kg. In some embodiments, the formulation has an osmolality of about <675 mOsm/kg. In some embodiments, the formulation has an osmolality of about <650 mOsm/kg. In some embodiments, the formulation has an osmolality of about <625 mOsm/kg. In some embodiments, the formulation has an osmolality of about <600 mOsm/kg. In some embodiments, the formulation has an osmolality of about <575 mOsm/kg. In some embodiments, the formulation has an osmolality of about <550 mOsm/kg. In some embodiments, the formulation has an osmolality of about <525 mOsm/kg. In some embodiments, the formulation has an osmolality of about <500 mOsm/kg. In some embodiments, the formulation has an osmolality of about <450 mOsm/kg. In some embodiments, the formulation has an osmolality of about <400 mOsm/kg. In some embodiments, the formulation has an osmolality of about <350 mOsm/kg. In some embodiments, the formulation has an osmolality of about <300 mOsm/kg. In some embodiments, the formulation has an osmolality of about ≤250 mOsm/kg.

In some embodiments, the formulation has an osmolality of about 300 mOsm/kg to about 850 mOsm/kg. In some embodiments, the formulation has an osmolality of about 300 mOsm/kg to about 350 mOsm/kg, about 300 mOsm/kg to about 400 mOsm/kg, about 300 mOsm/kg to about 450 mOsm/kg, about 300 mOsm/kg to about 500 mOsm/kg, about 300 mOsm/kg to about 550 mOsm/kg, about 300 mOsm/kg to about 600 mOsm/kg, about 300 mOsm/kg to about 650 mOsm/kg, about 300 mOsm/kg to about 700 mOsm/kg, about 300 mOsm/kg to about 750 mOsm/kg, about 300 mOsm/kg to about 800 mOsm/kg, about 300 mOsm/kg to about 850 mOsm/kg, about 350 mOsm/kg to about 400 mOsm/kg, about 350 mOsm/kg to about 450 mOsm/kg, about 350 mOsm/kg to about 500 mOsm/kg, about 350 mOsm/kg to about 550 mOsm/kg, about 350 mOsm/kg to about 600 mOsm/kg, about 350 mOsm/kg to about 650 mOsm/kg, about 350 mOsm/kg to about 700 mOsm/kg, about 350 mOsm/kg to about 750 mOsm/kg, about 350 mOsm/kg to about 800 mOsm/kg, about 350 mOsm/kg to about 850 mOsm/kg, about 400 mOsm/kg to about 450 mOsm/kg, about 400 mOsm/kg to about 500 mOsm/kg, about 400 mOsm/kg to about 550 mOsm/kg, about 400 mOsm/kg to about 600 mOsm/kg, about 400 mOsm/kg to about 650 mOsm/kg, about 400 mOsm/kg to about 700 mOsm/kg, about 400 mOsm/kg to about 750 mOsm/kg, about 400 mOsm/kg to about 800 mOsm/kg, about 400 mOsm/kg to about 850 mOsm/kg, about 450 mOsm/kg to about 500 mOsm/kg, about 450 mOsm/kg to about 550 mOsm/kg, about 450 mOsm/kg to about 600 mOsm/kg, about 450 mOsm/kg to about 650 mOsm/kg, about 450 mOsm/kg to about 700 mOsm/kg, about 450 mOsm/kg to about 750 mOsm/kg, about 450 mOsm/kg to about 800 mOsm/kg, about 450 mOsm/kg to about 850 mOsm/kg, about 500 mOsm/kg to about 550 mOsm/kg, about 500 mOsm/kg to about 600 mOsm/kg, about 500 mOsm/kg to about 650 mOsm/kg, about 500 mOsm/kg to about 700 mOsm/kg, about 500 mOsm/kg to about 750 mOsm/kg, about 500 mOsm/kg to about 800 mOsm/kg, about 500 mOsm/kg to about 850 mOsm/kg, about 550 mOsm/kg to about 600 mOsm/kg, about 550 mOsm/kg to about 650 mOsm/kg, about 550 mOsm/kg to about 700 mOsm/kg, about 550 mOsm/kg to about 750 mOsm/kg, about 550 mOsm/kg to about 800 mOsm/kg, about 550 mOsm/kg to about 850 mOsm/kg, about 600 mOsm/kg to about 650 mOsm/kg, about 600 mOsm/kg to about 700 mOsm/kg, about 600 mOsm/kg to about 750 mOsm/kg, about 600 mOsm/kg to about 800 mOsm/kg, about 600 mOsm/kg to about 850 mOsm/kg, about 650 mOsm/kg to about 700 mOsm/kg, about 650 mOsm/kg to about 750 mOsm/kg, about 650 mOsm/kg to about 800 mOsm/kg, about 650 mOsm/kg to about 850 mOsm/kg, about 700 mOsm/kg to about 750 mOsm/kg, about 700 mOsm/kg to about 800 mOsm/kg, about 700 mOsm/kg to about 850 mOsm/kg, about 750 mOsm/kg to about 800 mOsm/kg, about 750 mOsm/kg to about 850 mOsm/kg, or about 800 mOsm/kg to about 850 mOsm/kg. In some embodiments, the formulation has an osmolality of about 300 mOsm/kg, about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, about 800 mOsm/kg, or about 850 mOsm/kg. In some embodiments, the formulation has an osmolality of at least about 300 mOsm/kg, about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, or about 800 mOsm/kg. In some embodiments, the formulation has an osmolality of at most about 350 mOsm/kg, about 400 mOsm/kg, about 450 mOsm/kg, about 500 mOsm/kg, about 550 mOsm/kg, about 600 mOsm/kg, about 650 mOsm/kg, about 700 mOsm/kg, about 750 mOsm/kg, about 800 mOsm/kg, or about 850 mOsm/kg.

In some embodiments, the formulation is isotonic.

In some embodiments, the formulation has an osmolality of about 500 mOsm/kg.

### Stability

In some embodiments, the formulation remains stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the formulation remains stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the concentration of R(−)-ketamine in the formulation is stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the concentration of R(−)-ketamine in the formulation is stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the impurities in the formulation are stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the impurities in the formulation are stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the osmolality in the formulation is stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the osmolality in the formulation is stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the pH of the formulation is stable for more than about four weeks, about five weeks, about six weeks, about seven weeks, about eight weeks, about nine weeks, about ten weeks, about eleven weeks, about twelve weeks, about thirteen weeks, about fourteen weeks, or about fifteen weeks.

In some embodiments, the pH of the formulation is stable for more than about 1 month, 2 months, 3 months, 4 months, 5 months, 6 months, 7 months, 8 months, 9 months, 10 months, 11 months, about 12 months, 13 months, 14 months, 15 months, 16 months, 17 months, 18 months, 19 months, 20 months, 21 months, 22 months, 23 months, or 24 months.

In some embodiments, the formulation is stable in the presence of light. In some embodiments, the formulation is stable in the presence of heat. In some embodiments, the formulation is stable at room temperature.

In some embodiments, the term stable means the percentage, volume, or concentration of impurities, pH, R(−)-ketamine, osmolality, etc. does not change more than about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% from the original percentage, volume, or concentration.

### Carrier Fluid

In some embodiments, the R(−)-ketamine subcutaneous formulation comprises a carrier liquid. In some embodiments, the R(−)-ketamine subcutaneous formulation comprises a carrier liquid, wherein the carrier fluid is water. In some embodiments, the R(−)-ketamine subcutaneous formulation comprises a carrier liquid, wherein the carrier fluid is water for injection (WFI). In some embodiments, the R(−)-ketamine subcutaneous formulation comprises a carrier liquid, wherein the carrier fluid is a buffering agent.

In some embodiments, the R(−)-ketamine subcutaneous formulation comprises water for injection. In some embodiments, the R(−)-ketamine subcutaneous formulation comprises water for injection and a buffer. In some embodiments, the R(−)-ketamine subcutaneous formulation comprises a buffer.

In some embodiments, the R(−)-ketamine subcutaneous formulation comprises a succinate buffer. In some embodiments, the R(−)-ketamine subcutaneous formulation comprises a tartrate buffer. In some embodiments, the R(−)-ketamine subcutaneous formulation comprises a maleate buffer. In some embodiments, the R(−)-ketamine subcutaneous formulation comprises a fumarate buffer. In some embodiments, the R(−)-ketamine subcutaneous formulation comprises a citrate buffer. In some embodiments, the R(−)-ketamine subcutaneous formulation comprises an acetate buffer. In some embodiments, the R(−)-ketamine subcutaneous formulation comprises water for injection and a maleate buffer. In some embodiments, the R(−)-ketamine subcutaneous formulation comprises water for injection and a fumarate buffer. In some embodiments, the R(−)-ketamine subcutaneous formulation comprises water for injection and a citrate buffer. In some embodiments, the R(−)-ketamine subcutaneous formulation comprises water for injection and an acetate buffer.

### Processes for Preparing the Formulation

In some embodiments, R(−)-ketamine, or pharmaceutically acceptable salt thereof, is added to water for injection and the pH is adjusted slowly using a dilute base such that the final concentration of R(−)-ketamine is between about 60 mg/mL and about 110 mg/mL. In some embodiments, the R(−)-ketamine used to prepare the formulation is selected from any of the salts or salt forms described in International Application No. PCT/US2022/046412. In some embodiments, a particular amount of the R(−)-ketamine, R(−)-ketamine salt, or R(−)-ketamine salt form is added to obtain the free base equivalents as disclosed herein.

In some embodiments, the pH of the R(−)-ketamine formulation is increased using an inorganic base. In some embodiments, the pH of the R(−)-ketamine formulation is increased using an inorganic base at a concentration of IN. In some embodiments, the pH of the R(−)-ketamine formulation is increased using an inorganic base at a concentration of 0.5N. In some embodiments, the pH of the R(−)-ketamine formulation is increased using an inorganic base at a concentration of 0.15N. In some embodiments, the pH of the R(−)-ketamine formulation is titrated.

In some embodiments, the process discussed herein for preparing a formulation for subcutaneous administration comprising R(−)-ketamine facilitates increased solubility of the R(−)-ketamine. In some embodiments, the pH is adjusted slowly. In some embodiments, the pH is adjusted slowly with a dilute acid or a dilute base. In some embodiments, the pH is adjusted in a way that minimizes localized fluctuations of pH. In some embodiments, the pH is adjusted such that micro-changes in pH in the solution is minimized. In some embodiments, the pH is adjusted slowly using a dilute acid or a dilute base such that micro-changes in pH in the solution is minimized. In some embodiments, the pH is adjusted slowly using a dilute acid or a dilute base such that micro-changes in pH in the solution is minimized which improves solubility of the R(−)-ketamine in the formulation.

The injectable compositions of the present disclosure can be manufactured by the skilled person by use of standard methods and conventional techniques appropriate to the desired formulation. The formulation for intramuscular or subcutaneous administration of the present disclosure can be packed and/or stored in a suitable container, including, without limitation, syringes, ampoules, vials, including sealed vials such as vials the openings of which are sealed with syringe pierceable septa or sure-seals caps, and the like. In some embodiments, the formulation is pre-filled in disposable syringes for self-administration by patients, with or without an auto-injector. Each container can contain R(−)-ketamine or a pharmaceutically acceptable salt thereof in desired dosage amounts.

To further minimize oxidative degradation of the active ingredients, the container may be filled with an inert gas, such as nitrogen and/or carbon dioxide, which is otherwise oxygen-free. It is further contemplated that the container(s) may be enclosed within a sealed package, from which oxygen has been excluded. This may be accomplished by vacuum packaging or by displacing oxygen with a blanket or purge of nitrogen, carbon dioxide, or other oxygen-free inert gas. After sealing the seal, the packaging materials themselves should be relatively impermeable to the diffusion of oxygen. Also, the packages should be opaque to ordinary light, as light may induce the decomposition of R(−)-ketamine. Standard methods for sealing and packaging the various containers described herein are well known in the art and can be used in conjunction with packaging and/or storing the dosage forms of the present disclosure.

In some embodiments, the R(−)-ketamine formulation is contained in a “unit dosage form.” The phrase “unit dosage form” refers to physically discrete units, each unit including a predetermined amount of R(−)-ketamine, sufficient to produce the desired effect. It will be appreciated that the parameters of a unit dosage form will depend on the concentration of R(−)-ketamine in the formulation, and the effect to be achieved.

R(−)-ketamine Formulations in Maleate Buffer

R(−)-ketamine formulations at various pH values were prepared in maleate buffer.

### Maleate Buffer Components:

Maleic acid, C₄H₄O₄, m.w. 116.07
NaOH, 1 N aqueous
Distilled H₂O
   The maleate buffer (100 mL at 20 mM) is prepared for example, by adding 0.232 g maleic acid, 2 mL NaOH, and by adding H₂O to 100 mL.
   R(−)-ketamine Formulation (40 mg mL) at pH 5.0 in Maleate Buffer 20 mM and R(−)-ketamine Formulation (40 mg mL) at pH 5.7 in Maleate Buffer 20 mM

The formulation is prepared by adding, for example, 40 mg/ml R(−)-ketamine free base equivalent (46 mg/ml R(−)-ketamine HCl salt) in 20 mM maleate buffer and the pH of the final formulation is adjusted using HCl or NaOH (below 0.2M or 0.15M) to equal a pH of 5.0 or a pH of 5.7.

R(−)-ketamine Formulation (80 mg mL) at pH 5.0 in Maleate Buffer 20 mM and R(−)-ketamine Formulation (80 mg mL) at pH 5.7 in Maleate Buffer 20 mM

The formulation is prepared by adding, for example, 80 mg/ml R(−)-ketamine free base equivalent (92 mg/ml R(−)-ketamine HCl salt) in 20 mM maleate buffer and the pH of the final formulation is adjusted using HCl or NaOH (below 0.2M or 0.15M) to equal a pH of 5.0 or a pH of 5.7.

R(−)-ketamine Formulations in Fumarate Buffer

R(−)-ketamine formulations at various pH values were prepared in fumarate buffer.

### Fumarate Buffer Components:

Fumaric acid, C₄H₄O₀₄, m.w. 116.07
NaOH, 1 N aqueous
Distilled H₂O
   The fumarate buffer (100 mL at 20 mM) is prepared for example, by adding 0.232 g fumaric acid, 2 mL NaOH, and by adding H₂O to 100 mL.
   R(−)-ketamine Formulation (40 mg mL) at pH 5.0 in Fumarate Buffer 20 mM and R(−)-ketamine Formulation (40 mg mL) at pH 5.7 in Fumarate Buffer 20 mM

The formulation is prepared by adding, for example, 40 mg/ml R(−)-ketamine free base equivalent (46 mg/ml R(−)-ketamine HCl salt) in 20 mM fumarate buffer and the pH of the final formulation is adjusted using HCl or NaOH (below 0.2M or 0.15M) to equal a pH of 5.0 or a pH of 5.7.

R(−)-ketamine Formulation (80 mg mL) at pH 5.0 in Fumarate Buffer 20 mM and R(−)-ketamine Formulation (80 mg mL) at pH 5.7 in Fumarate Buffer 20 mM

The formulation is prepared by adding, for example, 80 mg/ml R(−)-ketamine free base equivalent (92 mg/ml R(−)-ketamine HCl salt) in 20 mM fumarate buffer and the pH of the final formulation is adjusted using HCl or NaOH (below 0.2M or 0.15M) to equal a pH of 5.0 or a pH of 5.7.

R(−)-ketamine Formulations in Citrate Buffer

R(−)-ketamine formulations at various pH values were prepared in citrate buffer, and the citrate buffer was prepared accordingly.

### Prepare 1 Liter Citrate Buffer 20 mM pH 5

| Component | Amount | | Concentration |
|---|---|---|---|
| Sodium Citrate dihydrate (mw: 294.10 | 3.42 | g | 0.0116M |
| g/mol) | | | |
| Citric Acid (mw: 192.12 g/mol) | 1.608 | g | 0.0084M |

1. Prepare 800 mL of distilled water in a suitable container.
2. Add 3.42 g of sodium citrate dihydrate to the solution.
3. Add 1.608 g of citric acid to the solution.
4. Adjust solution to final desired pH using HCl or NaOH
5. Add distilled water until volume is 1 L.

### Prepare 1 Liter Citrate Buffer at pH 5.7

| Component | Amount | | Concentration |
|---|---|---|---|
| Sodium Citrate dihydrate (mw: 294.10 | 4.424 | g | 0.015M |
| g/mol) | | | |
| Citric Acid (mw: 192.12 g/mol) | 953 | mg | 0.005M |

1. Prepare 800 mL of distilled water in a suitable container.
2. Add 4.424 g of sodium citrate dihydrate to the solution.
3. Add 0.953 g of citric acid to the solution.
4. Adjust solution to final desired pH using HCl or NaOH
5. Add distilled water until volume is 1 L.
   R(−)-ketamine Formulation (40 mg mL) at pH 5.0 in Citrate Buffer 20 mM and R(−)-ketamine Formulation (40 mg mL) at pH 5.7 in Citrate Buffer 20 mM

The formulation is prepared by adding, for example, 40 mg/ml R(−)-ketamine free base equivalent (46 mg/ml R(−)-ketamine HCl salt) in 20 mM citrate buffer and the pH of the final formulation is adjusted using HCl or NaOH (below 0.2M or 0.15M) to equal a pH of 5.0 or a pH of 5.7.

R(−)-ketamine Formulation (80 mg mL) at pH 5.0 in Citrate Buffer 20 mM and R(−)-ketamine Formulation (80 mg mL) at pH 5.7 in Citrate Buffer 20 mM

The formulation is prepared by adding, for example, 80 mg/ml R(−)-ketamine free base equivalent (92 mg/ml R(−)-ketamine HCl salt) in 20 mM citrate buffer and the pH of the final formulation is adjusted using HCl or NaOH (below 0.2M or 0.15M) to equal a pH of 5.0 or a pH of 5.7.

R(−)-ketamine Formulations in Succinate Buffer

R(−)-ketamine formulations at various pH values were prepared in succinate buffer.

### Succinate Buffer Components:

Succinic acid, C₄H₄O₀₄, m.w. 118.09
NaOH, 1 N aqueous
Distilled H₂O
   The fumarate buffer (100 mL at 20 mM) is prepared for example, by adding 0.236 g succinic acid, 2 mL NaOH, and by adding H₂O to 100 mL.
   R(−)-ketamine Formulation (40 mg mL) at pH 5.0 in Succinate Buffer 20 mM and R(−)-ketamine Formulation (40 mg mL) at pH 5.7 in Succinate Buffer 20 mM

The formulation is prepared by adding, for example, 40 mg/ml R(−)-ketamine free base equivalent (46 mg/ml R(−)-ketamine HCl salt) in 20 mM succinate buffer and the pH of the final formulation is adjusted using HCl or NaOH (below 0.2M or 0.15M) to equal a pH of 5.0 or a pH of 5.7.

R(−)-ketamine Formulation (80 mg mL) at pH 5.0 in Succinate Buffer 20 mM and R(−)-ketamine Formulation (80 mg mL) at pH 5.7 in Succinate Buffer 20 mM

The formulation is prepared by adding, for example, 80 mg/ml R(−)-ketamine free base equivalent (92 mg/ml R(−)-ketamine HCl salt) in 20 mM succinate buffer and the pH of the final formulation is adjusted using HCl or NaOH (below 0.2M or 0.15M) to equal a pH of 5.0 or a pH of 5.7.

R(−)-ketamine Formulations in Tartrate Buffer

R(−)-ketamine formulations at various pH values were prepared in tartrate buffer.

### Tartrate Buffer Components:

Tartaric acid, C₄H₆O₆, m.w. 150.09
Sodium tartrate dihydrate, m.w. 230.08
NaOH, 1 N aqueous
Distilled H₂O

The tartrate buffer (1 L at 5 mM) is prepared for example, by adding 0.75 g tartaric acid, 2 mL NaOH, and by adding H₂O to 1 L. The tartrate buffer (1 L at 15 mM) is prepared for example, by adding 3.452 g sodium tartrate dihydrate, 2 mL dil. acid, and by adding H₂O to 1 L. The tartrate buffer (1 L at 20 mM) is prepared for example, by adding 4.6 g sodium tartrate dihydrate, 2 mL dil. acid, and by adding H₂O to 1 L.

R(−)-ketamine Formulation (40 mg/mL) at pH 5.0 in Tartrate Buffer 20 mM and R(−)-ketamine Formulation (40 mg/mL) at pH 5.7 in Tartrate Buffer 20 mM

The formulation is prepared by adding, for example, 40 mg/ml R(−)-ketamine free base equivalent (46 mg/ml R(−)-ketamine HCl salt) in 20 mM tartrate buffer and the pH of the final formulation is adjusted using HCl or NaOH (below 0.2M or 0.15M) to equal a pH of 5.0 or a pH of 5.7.

R(−)-ketamine Formulation (80 mg/mL) at pH 5.0 in Tartrate Buffer 20 mM and R(−)-ketamine Formulation (80 mg/mL) at pH 5.7 in Tartrate Buffer 20 mM

The formulation is prepared by adding, for example, 80 mg/ml R(−)-ketamine free base equivalent (92 mg/ml R(−)-ketamine HCl salt) in 20 mM tartrate buffer and the pH of the final formulation is adjusted using HCl or NaOH (below 0.2M or 0.15M) to equal a pH of 5.0 or a pH of 5.7.

### Methods of Treating Diseases and Disorders

The methods disclosed herein can be used to treat a variety of diseases and disorders. In some cases, the disease or disorder is associated with depressive symptoms, and the methods of administering R(−)-ketamine compositions described herein can be used to treat the depressive symptom.

In some embodiments, the depressive symptom is a symptom of a mood disorder in the subject. Exemplary mood disorders include depression, such as treatment resistant depression or major depressive disorder. Further exemplary mood disorders include bipolar disorder, post traumatic stress disorder, obsessive compulsive disorder, autism spectrum disorder, schizophrenia, or dementia. Additional diseases or disorders are also envisaged as within the scope of the instant disclosure, for example neurodegenerative disease or disorders, a neurodevelopmental disorders, and an inflammatory or bone diseases. Exemplary diseases that can be treated with the R(−)-ketamine compositions and methods of using same disclosed herein are described in WO/2015/037248, WO 2019/213551, WO 2019/065900, WO 2019/160057, and WO 20200138491, the contents of each of which are incorporated by reference herein.

In some embodiments, the neurodegenerative disease or disorder comprises Parkinson's disease, Parkinson's syndrome, Huntington's disease, spiny erythrocyte chorea, spinal cord cerebellar degeneration, amyotrophic lateral sclerosis, spinal muscular atrophy, primary lateral sclerosis, spinal and bulbar muscular atrophy, syringomyelia, neurospinous erythrocytosis, eating disorders, Alzheimer's disease, Lewy body dementia, basal ganglia degeneration, multiple sclerosis, traumatic brain injury, cerebral infarction, or cardiovascular disease.

In some embodiments, the neurodevelopmental disorder comprises schizophrenia, autism spectrum disorder, attention-deficit/hyperactivity disorder, or a learning disorder.

In some embodiments, the inflammatory disease comprises ulcerative colitis, Crohn's disease, rheumatoid arthritis, ankylosing spondylitis, insulin-dependent diabetes, Addison's disease, Goodpasture syndrome, IgA nephropathy, interstitial nephritis, Sjögren's syndrome, autoimmune pancreatitis, psoriasis, atopic dermatitis, pneumonia, chronic bronchitis, bronchial asthma, systemic lupus erythematosus (SLE), scleroderma, or delirium, and the bone disease comprises osteoporosis, osteolytic bone metastasis, or Paget's disease of bone.

### Mood Disorders

In some embodiments, the methods of the present disclosure further comprise treating a mood disorder in the subject. As use herein, “mood disorder” refers to a mental health disorder with a serious change in mood. Exemplary mood disorders include, but are not limited to, treatment-resistant depression (TRD), major depressive disorder, bipolar disorder, dementia, low motivation, anxiety, insomnia, anorexia, obsessive compulsive disorder, post traumatic stress disorder, persistent depressive disorder, cyclothymia and seasonal affective disorder. Symptoms of mood disorders include, but are not limited to, anhedonia or negative affect, mood depression, low motivation, anxiety, insomnia, anorexia, impulsivity and stress sensitivity. In some embodiments, the methods disclosed herein comprise treating or ameliorating TRD. TRD may be defined as a depressive disorder that does not respond to treatment with two or more antidepressant regimens.

Treatment of Substance Abuse with R(−)-ketamine

Racemic ketamine has been shown to have potential in helping mitigate withdrawal symptoms in patients that arise after chronic opioid use (Non-Patent Literature 38), and data from animal models have suggested that racemic ketamine might help dampen the addiction-related phenomena of drug tolerance and dependence for both opiates and ethanol (Non-Patent Literature 39, 40, 41). However, racemic ketamine also has a number of associated problems that make it a troubling choice for administration to patients, as described elsewhere herein. Racemic ketamine is also itself known to be potentially addictive, making it a troubling choice for the treatment of substance use disorders (Non-Patent Literature 42, 43, 44). In addition, racemic ketamine administration is associated with damage to the central nervous system, especially after chronic use (Non-Patent Literature 45). Therefore, safer alternatives to racemic ketamine are needed.

The inventors of the present disclosure conducted studies indicating that R(−)-ketamine offers a superior treatment for substance use disorders such as opioid and alcohol use disorders when compared to S(+)-ketamine and racemic ketamine. For instance, in a mouse model of morphine addiction, R(−)-ketamine significantly attenuated the addictive properties of morphine when co-administered to the mice and R(−)-ketamine administered to rats significantly ameliorated the symptoms of precipitated withdrawal from morphine. R(−)-ketamine also mitigated the effects of alcohol tolerance in rats. Surprisingly, R(−)-ketamine and its metabolite R-hydroxynorketamine, unlike S(+)-ketamine, also showed no anhedonic or negative affective side effects in rats.

### Substances of Abuse

The abuse of drugs is linked to their ability to produce specific subjective effects in humans (e.g., euphoria). There are many substances which can lead to a substance use disorder in a subject. As used herein, the term “drug” means a substance which may cause addiction or dependence upon continuous use. Both legal and illegal (illicit) substances can lead to substance use disorders. Included within this term are drugs such as alcohol, marijuana, synthetic cannabinoids, opioids, stimulants, barbiturates, benzodiazepines, dextromethorphan (DXM), sleep medications, khat, synthetic cathinones, cocaine, 3,4-methylenedioxymethamphetamine (MDMA), phencyclidine (PCP), lysergic acid diethylamide (LSD), psilocybin, inhalants, Rohypnol, gamma-hydroxybutyric acid (GHB), N,N-Dimethyltryptamine (DMT), ayahuasca, mescaline, salvia and nicotine. However, any substance which may cause addiction or dependence is envisaged as being within the scope of the invention.

Most substances fall within three major categories: stimulants, depressants and hallucinogens or dissociative substances. In some embodiments, in particular, but not limited to, those embodiments wherein the substance is a complex botanical product such as marijuana, the substance may have more than one active ingredient and fall into more than one of the three categories. In some embodiments, the substance may have a single active ingredient with multiple effects, and thus be classified in more than one of the three categories. In some embodiments, the substance may be classified in a single category as a stimulant, depressant, or a hallucinogenic or dissociative substance.

Substances of the disclosure can be naturally occurring, for example purified from a plant, animal or fungal source (marijuana, tobacco, e.g.), can be synthetic (synthetic cathinones, LSD, e.g.), or a combination thereof. A substance of the disclosure can be a synthetic version of substance originally purified from a natural source. A list of exemplary, but non-limiting substances of the disclosure is set forth in Table 1. All substances, and all biological mechanisms, capable of inducing a substance use disorder in a subject are envisaged as within the scope of the present invention.

TABLE 1
List of Substances that can lead to substance use disorders

| | | |
|---|---|---|
| Name | Brand Name ® | Additional Names/Information |

Depressants

| | | |
|---|---|---|
| alcohol | | Ethanol |
| phenobarbital | Luminal | (barbiturate) |
| pentobarbital | Nembutal | (barbiturate) |
| methohexital | Brevital, Brietal | (barbiturate) |
| butabarbital | Butisol | (barbiturate) |
| butalbital | Fioricet (with acetaminophen | (barbiturate) |
| | and caffeine) | |
| alprazolam | Xanax, Alprazolam Intensol | (benzodiazepine) |
| chlorodiazepoxide | Librium | (benzodiazepine) |
| diazepam | Valium, Diastat, Diazepam | (benzodiazepine) |
| | Intensol | |
| lorazepam | Ativan, Lorazepam Intensol | (benzodiazepine) |
| triazolam | Halcion | (benzodiazepine) |
| Rohypnol | Flunitrazepam, Rohypnol | (benzodiazepine) |
| eszopiclone | Lunesta | (sleep medication) |
| zaleplon | Sonata | (sleep medication) |
| zolpidem | Ambien, Edluar, Intermezzo, | (sleep medication) |
| | Zolpimist | |
| GHB, Gamma- | Xyrem | γ-Hydroxybutyric acid; 4- |
| hydroxybutyrate | | hydroxybutanoic acid; sodium |
| | | oxybate |

Hallucinogens and Dissociatives

| | | |
|---|---|---|
| ayahusca | | Psychotria viridis |
| DMT | | N,N-Dimethyltryptamine |
| LSD | | D-lysergic acid diethylamide |
| mescaline | | (3,4,5-trimethoxyphenethylamine; |
| | | Peyote - Cactaceae family |
| psilocybin | | psilocybin mushrooms |
| salvia | | Salvia divinorum |
| PCP | | Phencyclidine; 1-(1- |
| | | Phenylcyclohexyl)piperidine |
| DXM | Wal-Tussin Cough, | Dextromethorphan |
| | Creomulsion, Vicks DayQuil | |
| | Cough, Cough DM ER, Tussin | |
| | Cough, Cough Relief, | |
| | Robitussin ER, Delsym, Scot- | |
| | Tussin Diabetes CF | |
| Kratom | | Mitragyna speciosa |

Stimulants

| | | |
|---|---|---|
| cocaine | | from Erythroxylon coca |
| Amphetamine | Adderall, Benzedrine, Evekeo | Amphetamine sulfate |
| Methamphetamine | Desozyn | |
| Dextroamphetamine | ProCentra, Dexedrine Spansule, | |
| | Zenzedi, Adderall, Benzedrine, | |
| | Evekeo | |
| Levoamphetamine | Adderall, Benzedrine, Evekeo | |
| Lisdexamfetamine | Vyvanse | |
| Atomoxetine | Strattera | |
| Methylphenidate | Concerta, Ritalin, Daytrana, | |
| | Methylin, Metadate, Aptensio, | |
| | Quillivant | |
| Dexmethylphenidate | Focalin, FocalinXR | |
| Oxymetazoline | Afrin, Duamist, Mucinex, | |
| | Nasacon, Nasin, Ocuclear, | |
| | Drixine | |
| Pseudoephedrine | Sudafed, Valu-Tapp, Sinus 12 | |
| | Hour, Wal-phed, Suphedrin | |
| Phenylephrine | Preparation H, Vazculep | |

Opioids

| | | |
|---|---|---|
| Heroin | | from poppy, papaver |
| | | somniferum; aka diamorphine |
| mitragynine | | from Mitragyna speciosa |
| Codeine | | (5α,6α)-7,8-didehydro-4,5-epoxy- |
| | | 3-methoxy-17-methylmorphinan- |
| | | 6-ol |
| Fentanyl | Actiq, Duragesic, Sublimaze, | |
| | Subsys, Abstral, Ionsys | |
| Hydrocodone, | Vicodin, Norco, Zohydro, | |
| dihydrocodeinone | Lorcet, Hycet, Zamicet, Xodol | |
| Hydromorphone | Dilaudid, Exalgo ER | |
| Meperidine | Demerol | |
| Methadone | Dolophine, Methadose | |
| Morphine | Duramorph, MS Contin, | |
| | Infumorph P/F, Arymo, | |
| | Astramorph-PF | |
| Oxycodone | Oxycontin, Roxicodone, | |
| | Percodan, Percocet | |
| Oxymorphone | Opana | |

Inhalants

| | |
|---|---|
| aerosols | e.g. spray deodorants, hair spray, |
| | insect repellant |
| gases | e.g. nitrous oxide |
| solvents | e.g. toluene, gasoline, paint |
| | thinner, dry cleaning fluids |
| nitrites | e.g. isoamyl nitrite, isobutyl nitrite, |
| | cyclohexyl nitrite |

Other/Mixed Classification

| | | |
|---|---|---|
| Marijuana | | Cannabis sativa |
| synthetic marijuana | | |
| cannabicyclohexanol | | |
| THC | Marinol, Syndros | delta-9-tetrahydrocannabinol; |
| | | Dronabinol; from Cannabis sativa |
| MDMA | | 3,4-methylenedioxy- |
| | | methamphetamine |
| Loperamide | Immodium | 4-[4-(4-chlorophenyl)-4- |
| | | hydroxypiperidin-1-yl]-N,N- |
| | | dimethyl-2,2-diphenylbutanamide |
| Khat | | Catha edulis |
| mephedrone | | 4-methyl methcathinone or 4- |
| | | methyl ephedrone, |
| methylone | | 2-Methylamino-1-(3,4- |
| | | methylenedioxyphenyl)propan-1- |
| | | one; MDMC |
| MDPV | | 3,4-methylenedioxypyrovalerone |
| tobacco | | Nicotiana |
| Nicotine | Nioctrol, Nicoderm | 3-(1-methyl-2- |
| | | pyrrolidinyl)pyridine |

### Treatment of Substance Use Disorders

In some embodiments, the methods of the present disclosure further comprise treating a substance use disorder in the subject. Substance use disorders are characterized by progressively uncontrollable substance use that persists in spite of negative consequences (e.g., social, economic and/or medical consequences). Substance use disorders are marked by a transition from substance use that is well controlled, to a use that is unregulated and destructive. This transition can be abrupt or progressive in nature. Substance use disorders are characterized by addiction, or dependence, upon the substance. When a subject is dependent upon, or addicted to, a substance, this means that there is a physical, physiological or psychological reaction and/or interaction of the substance and the subject, which results in the subject exhibiting or having a forced or compulsive use of the substance without a recognized purpose or need for treating a disease. Rather, the purpose is that of achieving the desired effect, and/or avoiding withdrawal symptoms as defined hereinafter, which occur when the substance is discontinued or the amount used is reduced. Substance use disorders are sometimes referred to as “substance abuse”, and the substance or substances to which the subject is addicted or dependent upon are “abused”, for example opioid abuse or alcohol abuse.

In some embodiments, the substance use disorder comprises abuse of alcohol, marijuana, synthetic cannabinoids, opioids, stimulants, barbiturates, benzodiazepines, dextromethorphan (DXM), a sleep medication, khat, synthetic cathinones, cocaine, 3,4-methylenedioxymethamphetamine (MDMA), phencyclidine (PCP), lysergic acid diethylamide (LSD), psilocybin, an inhalant, Rohypnol, gamma-hydroxybutyric acid (GHB), N,N-Dimethyltryptamine (DMT), ayahuasca, mescaline, salvia, or nicotine.

In some embodiments, the opioid comprises Heroin, Codeine, Fentanyl, Hydrocodone (Dihydrocodeinone), Hydromorphone, Meperidine, Methadone, Morphine, Oxycodone or Oxymorphone. In some embodiments, the stimulant comprises Amphetamine, Amphetamine sulfate, Methamphetamine, Dextroamphetamine, Levoamphetamine, Lisdexamfetamine, Atomoxetine, Methylphenidate, Dexmethylphenidate, Oxymetazoline, Pseudoephedrine, Phenylephrine or a combination thereof. In some embodiments, the benzodiazepine comprises Aprazolam, Chlorodiazepoxide, Diazepam, Lorazepam or Triazolam. In some embodiments, the barbiturate comprises Phenobarbital, Pentobarbital, Methohexital, Secobarbital, Butabarbital or Butalbital. In some embodiments, the sleep medication comprises Eszopiclone, Zaleplon or Zolpidem.

In some embodiments, the therapeutically effective amount of the composition comprising R(−)-ketamine does not cause anhedonia or negative affect in the subject. Anhedonia and negative affect are symptoms associated with substance use disorders of the disclosure. As used herein, anhedonia, a clinical feature of mood disorders such as depression and bipolar disorder, refers to the reduction or loss of the capacity to experience pleasure. Negative affect refers to a preponderance of negative moods and emotions in a subject that are counter to well being. Anhedonia and negative affect are thought to be key factors involved in both relapse and the transition from recreation to excessive substance use. Without wishing to be bound by theory, anhedonia and negative affect are thought to originate in the dopaminergic mesolimbic and mesocortical reward circuit. Surprisingly, the R(−)-ketamine compositions and methods of the disclosure do not induce anhedonia or negative affect. Since subjects experience anhedonia and negative affect both while abusing substances in substance use disorders and while attempting to stop substance abuse, treatments that do not in and of themselves create or exacerbate anhedonia or negative affect are preferred and more effective treatments for substance use disorders.

In some embodiments, the substance use disorder comprises abuse of opioids. In some embodiments, the substance use disorder comprises abuse of alcohol.

In some embodiments, administering the composition reduces a symptom of withdrawal or prevents a relapse of the substance use disorder in the subject. In some embodiments, administering the composition reduces tolerance to a substance of the substance use disorder in the subject. In some embodiments, administering the composition reduces dependence on a substance of the substance use disorder in the subject. In some embodiments, administering the composition improves adherence to a treatment for the substance use disorder in the subject. In some embodiments, administering the composition reduces a preference for a substance of the substance use disorder or decreases liking for a substance the substance use disorder in the subject. In some embodiments, administering the composition increases abstinence from a substance of the substance use disorder in the subject.

In some embodiments, the method further comprises treating at least one substance use withdrawal symptom in the subject. Withdrawal occurs when a subject accustomed to a relatively stable level of a substance is suddenly deprived of that substance. Withdrawal symptoms may be physical or psychological in nature, or a combination thereof. The specific withdrawal symptoms may depend on the substance being withdrawn from, the amount and duration of substance use, and the individual. Withdrawal symptoms may be immediate or delayed in onset.

In some embodiments, the at least one substance use withdrawal symptom comprises a symptom of withdrawal from opioids. In some embodiments, the at least one substance use withdrawal symptom comprises a symptom of withdrawal from alcohol.

In some embodiments, the at least one substance use withdrawal symptom comprises a physical symptom of withdrawal, a psychological symptom of withdrawal or a combination thereof. In some embodiments, the physical symptom of withdrawal comprises tremors, insomnia, disturbed sleep, headache, sweating, nausea, vomiting, muscle pain, muscle stiffness, hypertension, irregular heart rate, elevated heart rate, heart palpitations, dizziness, shakiness, tremors, seizures, dehydration, shallow breathing, fatigue, loss of appetite, clammy skin, loss of color or a combination thereof. In some embodiments, the psychological symptom of withdrawal comprises anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing, a substance craving or a combination thereof.

In some embodiments, the method further comprises treating a psychological symptom associated with a substance use disorder in the subject. The symptoms of a substance use disorder depend upon the substance used, the duration and amount of use, and the subject. These symptoms can be physical or psychological in nature, or a combination thereof. Physical symptoms of substance use include, but are not limited to, tremors, insomnia, disturbed sleep, headache, sweating, nausea, vomiting, muscle pain, muscle stiffness, hypertension, irregular heart rate, elevated heart rate, heart palpitations, dizziness, shakiness, tremors, seizures, dehydration, shallow breathing, fatigue, loss of appetite, clammy skin, loss of color or a combination thereof. Psychological symptoms of substance use include, but are not limited to, anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing, craving or a combination thereof.

In some embodiments, the psychological symptom comprises a psychological symptom of a mood disorder that is comorbid with the substance use disorder. In some embodiments, the mood disorder comprises major depressive disorder, bipolar disorder, post traumatic stress disorder, obsessive compulsive disorder or dementia.

In some embodiments, the psychological symptom comprises anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing, a substance craving or a combination thereof.

In some embodiments, the composition is administered prior to the onset of the at least one psychological symptom in the subject. In some embodiments, the composition is administered at the same time as the onset of the at least one psychological symptom in the subject. In some embodiments, the composition is administered after the onset of the at least one psychological symptom in the subject. In some embodiments, the composition reduces or eliminates the at least one psychological symptom in the subject.

### Adverse Events

As used herein, the term “adverse event” is any untoward medical occurrence that occurs in the subject and is temporally associated with the use of the methods described herein. For example, an adverse event (AE) can be any unfavorable and unintended sign (e.g., an abnormal laboratory finding), symptom, or disease (new or exacerbated temporally associated with the use of the methods described herein. In some embodiments, the AE may be abnormal laboratory test results (hematology, clinical chemistry, or urinalysis) or other safety assessments (e.g., ECG, radiological scans, vital signs measurements); exacerbation of a chronic or intermittent pre-existing condition; new conditions detected or diagnosed after use of the methods described herein; signs, symptoms, or the clinical sequelae of a suspected drug-drug interaction; signs, symptoms, or the clinical sequelae of a suspected overdose of either the compositions described herein or a concomitant medication. As used herein, the term “serious adverse event” is any untoward medical occurrence that, at any dose: a) results in death; b) is life-threatening; c) requires inpatient hospitalization or prolongation of existing hospitalization; d) results in persistent disability/incapacity; or e) is a congenital anomaly/birth defect. In some embodiments, use of the methods described herein occurs in the absence of adverse events and serious adverse events. In some embodiments, the methods described herein reduce adverse events, compared to equivalent methods administering S(+)-ketamine or racemic ketamine.

### Kits and Articles of Manufacture

In another aspect, provided herein are kits comprising the R(−)-ketamine compositions of the embodiments described herein. In some embodiments, the kits further comprise packaging of the R(−)-ketamine compositions, and instructions for use.

The present description sets forth numerous exemplary configurations, methods, parameters, and the like. It should be recognized, however, that such description is not intended as a limitation on the scope of the present disclosure but is instead provided as a description of exemplary embodiments. Embodiments of the present subject matter described above may be beneficial alone or in combination, with one or more other aspects or embodiments. Without limiting the foregoing description, certain non-limiting embodiments of the disclosure are provided below. As will be apparent to those of skill in the art upon reading this disclosure, each of the embodiments may be used or combined with any of the preceding or following embodiments. This is intended to provide support for all such combinations of embodiments and is not limited to combinations of embodiments explicitly provided.

### EXAMPLES

### Example 1: Safety and Efficacy of R(−)-ketamine in Human Subjects

### A. Overview of Study

The present disclosure is a proof-of-concept study to assess efficacy and safety and provides evidence that R(−)-ketamine can be developed as a rapidly acting antidepressant for the potential treatment of treatment-resistant depression (TRD). To date, there has been one clinical trial that evaluated the safety of R(−)-ketamine. The primary objective of the study was to identify an acceptable tolerated dose of R(−)-ketamine in healthy subjects and compare this dose to the safety profile of 15 mg S(+)-ketamine, a dose which demonstrated a robust antidepressant effect in a published study of TRD patients (Non-Patent Literature 36). Dose ranging using single ascending doses of R(−)-ketamine demonstrated that doses of R(−)-ketamine≤150 mg had acceptable safety profiles as single 40-minute IV infusions. A comparison of the safety of dose levels of R(−)-ketamine and 15 mg S(+)-ketamine identified 60 mg R(−)-ketamine as the dose level most similar to 15 mg S(+)-ketamine in terms of safety profile. Both 30 mg and 60 mg doses of R(−)-ketamine resulted in fewer transient elevations in mean systemic blood pressure, less sedation, fewer psychological or psychotic and dissociative effects, and fewer episodes of altered consciousness than 100 mg or 150 mg doses of R(−)-ketamine.

The doses of 30 mg and 60 mg used in this study were selected based on the results from a Phase 1 single-ascending-dose study in healthy volunteers (Example 2, below). Due to its 4-fold lower affinity to the receptor, it is hypothesized that R(−)-ketamine may have fewer N-methyl-D-aspartate receptor (NMDAR)-related adverse effects compared with racemic ketamine or S(+)-ketamine, such as psychotomimetic effects and dissociation. R(−)-ketamine has a 15-fold higher affinity to the sigma receptor compared with S(+)-ketamine. Thus, ketamine's sigma-related adverse effects may be driven mostly by R(−)-ketamine. This action of R(−)-ketamine may play a role in the hallucinogenic effects of racemic ketamine, and it also may be responsible for the lowering of the seizure threshold seen with racemic ketamine.

Reported adverse events (AEs) from limited academic clinical studies in healthy volunteers evaluating R(−)-ketamine subanesthetic doses of up to 1 mg/kg IV as bolus or short infusion or 1.8 mg/kg intramuscular included transient increases in blood pressure, emotional changes, illusion, sedation, proprioceptive and sensory disturbances, decline in concentration capacity and primary memory, blurred vision, altered hearing, and dizziness. Overall, less pronounced psychotomimetic and dissociative-like effects were reported with R(−)-ketamine than with S(+)-ketamine (Non-Patent Literature 20, 21, 22, 23, 24, 25). Results from an open-label pilot study of a single IV infusion of R(−)-ketamine (0.5 mg/kg) administered to 7 subjects with TRD suggested that R-ketamine might produce fast-onset and sustained antidepressant effects with a favorable safety profile (Non-Patent Literature 35).

Ketamine has been reported being used as a drug of abuse. Ketamine dependence and tolerance are possible following prolonged administration. Although R(−)-ketamine may have a potential for abuse, nonclinical data suggest a lower abuse potential for R(−)-ketamine than that for racemic ketamine or S(+)-ketamine due to its pharmacological properties, such as lesser affinity to NMDAR and a lack of effects on the dopamine pathway (Non-Patent Literature 4, 30, 31, 32). The abuse and dependence potential of R(−)-ketamine in humans is unknown and has not yet been studied.

Multiple in vitro studies examining the in vitro drug metabolism and pharmacokinetics of R(−)-ketamine have been conducted. Multiple cytochrome P450 (CYP) isoforms are involved in the metabolism of R(−)-ketamine, primarily CYP2B6, CYP3A4/5, and CYP2C19. R(−)-ketamine is considered an inhibitor of CYP2B6 and CYP2C19, and an inducer of CYP1A2, CYP2B6, and CYP3A4. There is a potential for drug-drug interactions with CYP2B6 and CYP3A4 substrates. The clinical impact of these drug-drug interactions (DDIs) is thought to be minimal.

### B. Study Design

The Investigational Medicinal Product (IMP) R(−)-ketamine hydrochloride in sterile water for intravenous injection was investigated as an at-home treatment of TRD. The International Union of Pure and Applied Chemistry (IUPAC) name is of the IMP is (2R)-2-(2-chlorophenyl)-2-(methylamino)cyclohexan-1-one. Subjects received the solution at either 30 mg or 60 mg dose, or a placebo. The IMP or placebo was administered on Day 1 as a single IV infusion via an electronic infusion pump over 40 minutes. The duration of study participation for each subject was up to 29 days including a screening period, a 3-day in-clinic visit, and 2 follow-up visits. 101 subjects were enrolled in approximately 20 study centers in the EU and the US.

This was a double-blind, randomized, placebo-controlled, multicenter study comprised of 3 phases: screening (up to 2 weeks [day-15 to day-2]), in-clinic treatment (day-1 to day 2; including double-blind treatment [day 1]), and post-treatment follow-up (7 and 14 days after infusion on days 8 and 15, respectively). The study consisted of 3 arms: placebo, R(−)-ketamine solution (30 mg), and R(−)-ketamine solution (60 mg.) A total of 101 adult subjects with TRD were randomly allocated in cohorts of 33-35 subjects/arm to the 3 arms of the study in a blinded manner.

The subjects were randomized within 14 days of screening (Visit 1). Subjects were admitted to the clinic the evening prior to study treatment administration (day-1, Visit 2) and underwent baseline testing to ensure continued study eligibility. Study treatments were infused IV over 40 minutes the next morning (day 1, Visit 2). Starting immediately prior to dosing, subjects were monitored closely for safety.

Additionally, the subjects' alertness, mood, and other psychological parameters were assessed by clinician- and patient-completed scales and questionnaires. Subjects were discharged no earlier than 24 hours post-infusion and after the final in-clinic assessments were completed (day 2, Visit 2). Subjects were asked to return to the clinic approximately 6 days (day 8, Visit 3) and 13 days (day 15, Visit 4) after discharge to assess the safety and tolerability of the study treatments and to determine the durability of the antidepressant effect.

The study population included adult men and women, ages 18 to 65 years inclusive, who met the Diagnostic and Statistical Manual of Mental Disorders, Fifth Edition (DSM-V) diagnostic criteria for major depressive disorder (MDD) without psychotic features confirmed by the Mini-International Neuropsychiatric Interview (MINI). Based on published data from racemic ketamine, R(−)-ketamine may have the potential for adverse fetal effects if administered to pregnant women. Therefore, female subjects of childbearing potential were to be adequately protected from becoming pregnant and pregnant women were not to be enrolled.

### Primary Objectives and Estimands

To determine the efficacy of two doses (30 mg and 60 mg) of intravenous (IV) R(−)-ketamine hydrochloride, the doses were administered as an IV infusion over 40 minutes. These doses are within the range shown to be well-tolerated in a Phase 1 study in healthy volunteers. Two doses (30 mg and 60 mg) of IV R(−)-ketamine hydrochloride were compared with placebo to determine the most effective dose in improving depressive symptoms in subjects with TRD as assessed by change from baseline to 24 hours after the start of the infusion of R(−)-ketamine hydrochloride in the Montgomery Åsberg Depression Rating Scale (MADRS) total score. The 10-item clinician-administered MADRS was designed to be used in subjects with MDD to measure the overall severity of depressive symptoms. The MADRS scale is validated, reliable, and acceptable to regulatory health authorities as a primary scale to determine efficacy in major depression.

The primary estimand was defined as follows:

Population: Subjects with TRD in the full analysis set (FAS) and analyzed according to their randomized treatment. The analysis population includes all randomized subjects who receive any amount of study treatment and have at least one postbaseline assessment available. Variable: Change in total MADRS score from baseline at 24 hours after the start of infusion.

Intercurrent event (ICE): Incidents where a subject did not receive the full treatment infusion were to be considered an intercurrent event. The intercurrent event was handled using a treatment policy where all observed values were to be used regardless of occurrence of an intercurrent event. No imputation was performed in the primary efficacy analysis.

Population-level summary: Difference in mean change from baseline between each R(−)-ketamine group versus placebo.

Additional methodology will be specified in the Statistical Analysis Plan (SAP) for sensitivity and supplementary analyses for assessing the robustness of results. These sensitivity or supplementary analyses will explore different methods for handling intercurrent events and different assumptions for missing data.

### Secondary Objectives and Estimands:

To assess the proportion of subjects with a response (defined as ≥50% improvement in MADRS total score from pre-dose).

To assess the proportion of subjects with remission (defined as MADRS total score≤10).

To define changes in Hamilton Depression Rating Scale (HAM-D). The HAM-D is a multiple-item questionnaire designed to provide an indication of depression in adults and used as a guide to evaluate recovery. The questionnaire is designed to rate the severity of depression by probing mood, feelings of guilt, suicide ideation, insomnia, agitation or retardation, anxiety, weight loss, and somatic symptoms.

Generalized Anxiety Disorder 7-Item (GAD-7). The GAD-7, is a self-administered 7-item scale that is used to diagnose anxiety and has sensitivity and specificity as a screener for panic, social anxiety, and post-traumatic stress disorder.

Clinical Global Impression-Severity (CGI-S) and Clinical Global Impression-Improvement (CGI-I). The CGI-S and CGI-I scales allow the assessment of minimal clinically important differences using an anchor-based approach calculated from the global impressions of the clinician and the subject.

Quick Inventory of Depressive Symptomatology-16 Items (QIDS-SR-16). The QIDS-SR-16, is a 16-item, self-reported scale of depression that has very similar sensitivity to the Inventory of Depressive Symptomatology Self-Report-30 Items and the Hamilton Rating Scale for Depression-24 Items in detecting changes in symptoms of depression.

European Quality-5 Dimensions-3 Levels (EQ-5D-3 L). The EQ-5D-is a widely accepted, self-completion instrument to assess health-related quality of life for the domains of mobility, capacity for self-care, conduct of usual activities, pain/discomfort, and anxiety/depression.

To determine the safety and tolerability of two doses of R(−)-ketamine administered IV in subjects with TRD compared with placebo.

All secondary efficacy estimands were defined using the same population, ICE strategy, and population level summary (difference in means or proportions between each randomized treatment and placebo, as appropriate) as described above. The variables for the estimands were the following:

MADRS total score was assessed at 2 and 4 hours, 7 and 14 days after the start of the infusion (days 8 and 15, respectively). Note, for the 2-hour and 4-hour recall periods, the sleep and appetite items were not assessed.

Proportion of subjects with ≥50% improvement in MADRS total score at 24 hours, 7 days, and 14 days after start of infusion (days 8 and 15, respectively).

Proportion of subjects with a MADRS total score≤10 at 24 hours, 7 days, and 14 days after start of infusion (days 8 and 15, respectively).

Changes in HAM-D on day 8 and day 15 after start of infusion.

Change from baseline in GAD-7 by visit.

Change from baseline in CGI-S by visit and CGI-I (calculated from pre-dose CGI-S).

Change from baseline in QIDS-SR-16 by visit.

Change from baseline in EQ-5D-3 L by visit.

Safety as assessed by: Vital signs, 12-lead electrocardiogram (ECG), Oxygen saturation (SpO2), Clinical laboratory parameters, AEs, Modified Observer's Assessment of Alertness/Sedation (MOAA/S), Clinician-Administered Dissociative States Scale (CADSS), Brief Psychiatric Rating Scale-Modified 4 Components (BPRS+), 5-Dimensional Altered States of Consciousness Rating Scale (5D-ASC), Columbia Suicide Severity Rating Scale (C-SSRS).

### Inclusion Criteria

Subjects were eligible to be included in the study only if all of the following criteria applied:

Be capable of giving and give signed informed consent, which includes compliance with the requirements and restrictions listed in the Informed Consent Form (ICF) and in this protocol.

Be male or female 18 to 65 years of age inclusive at the time of signing the ICF.

Weigh ≥50 kg and have a body mass index (BMI) ≥18 and ≤35.

Have a diagnosis of recurrent major depressive disorder (MDD) without psychotic features per the Diagnostic and Statistical Manual of Mental Disorders, Fifth Edition (DSM-V), confirmed by the Mini-International Neuropsychiatric Interview (MINI).

Have an HAM-D total score >20 at screening and baseline (Day −1).

Have an inadequate response to at least 2 antidepressants in the current episode of depression that were each given for >6 weeks at an adequate dose as defined by the Massachusetts General Hospital Antidepressant Response Questionnaire (MGH-ATRQ).

Must be on stable oral antidepressant treatment without a dose change for at least 30 days before screening (a missed dose, or reasonable number of missed doses per Investigator's discretion, in that period does not exclude a subject).

A male subject must be medically confirmed sterile for at least 6 months prior to screening or agree to use highly effective contraception during the treatment period and for at least 3 months after the last dose of study treatment and refrain from donating sperm during this period. If a male with a partner who is of childbearing potential (OCBP) is included, his partner also needs to use highly effective birth control measures.

A female subject is eligible to participate if she is not pregnant, not breastfeeding, and at least 1 of the following conditions applies:

Not of childbearing potential. A subject is OCBP who agrees to follow the highly effective contraceptive guidance on highly effective birth control measures during the treatment period and for at least 3 months after the last dose of study treatment and refrain from donating eggs during this period.

Be medically stable on the basis of physical examination, medical history, vital signs, and 12-lead ECG performed at screening. If there are abnormalities, the subject may be included only if the Investigator judges the abnormalities not to be clinically important. This determination must be recorded in the subject's source documents and initialed and dated by the Investigator.

### Exclusion Criteria

Subjects were excluded from the study if any of the following criteria apply:

History of, or current signs and symptoms of, diseases or conditions that would make participation not be in the best interest (e.g., compromise the well-being) of the subject or that could prevent, limit, or confound the protocol-specified assessments.

History of moderate or severe head trauma (for example, loss of consciousness for more than 15 minutes) or other neurological disorders (including a diagnosis of epilepsy or has had a seizure in the last 6 months), neurodegenerative disorder (Alzheimer's disease, Parkinson's disease, multiple sclerosis, Huntington's disease, etc.) or systemic medical diseases that are, in the opinion of the Investigator, likely to interfere with the conduct of the study or confound the study assessments. A history of febrile seizures in childhood is not exclusionary.

Has a primary DSM-V diagnosis of current (active) MDD with psychotic features, panic disorder, obsessive compulsive disorder, posttraumatic stress disorder, anorexia nervosa, or bulimia nervosa. Comorbid anxiety or panic disorder that does not dominate the clinical presentation is acceptable.

Has a current or prior DSM-V diagnosis of a primary psychotic disorder (e.g., schizophrenia), bipolar or related disorders (confirmed by the MINI), intellectual or autism spectrum disorder, or borderline personality disorder.

Has any significant disease or disorder (e.g., cardiovascular, pulmonary, gastrointestinal, hepatic, renal, neurological, musculoskeletal, endocrine, metabolic, malignant, psychiatric, major physical impairment) that, in the opinion of the Investigator, may either put the subject at risk because of participation in the study, influence the results of the study, or affect the subject's ability to participate in the study.

Has uncontrolled hypertension, despite medication, at Screening (systolic blood pressure [SBP]>160 mm Hg or diastolic blood pressure [DBP]>90 mm Hg) or any past history of hypertensive crisis. An abnormal blood pressure value at screening may be repeated once after 10-15 minutes of relaxation to determine the subject's eligibility.

Has an abnormal ECG of clinical relevance at screening or baseline (Day-1) including, but not limited to, the following: QT interval corrected according to Fridericia's formula (QTcF) interval >450 msec for male subjects and >470 msec for female subjects; evidence of 2nd and 3rd degree atrioventricular block, complete left bundle branch block (LBBB), or complete right bundle branch block (RBBB); features of new ischaemia; arrhythmia (except premature atrial contractions [PACs] and premature ventricular contractions [PVCs]); or has a history of risk factors including hypokalemia or a family history of Long QT Syndrome.

Has known history of, or positive serology for, human immunodeficiency virus (HIV); has a positive hepatitis B surface antigen, and/or confirmed current hepatitis C infection (positive hepatitis C virus [HCV] antibody confirmed with reflex HCV ribonucleic acid [RNA] test). Subjects with a history of hepatitis B vaccination without a history of hepatitis B are allowed to enroll.

Has a history of malignancy within the 5 years prior to screening (exceptions are squamous and basal cell carcinomas of the skin and carcinoma in situ of the cervix, or a malignancy that is considered to have minimal risk of recurrence).

Has homicidal ideation/intent per the Investigator's clinical judgment; or has suicidal ideation with some intent to act within 1 month prior to the start of screening per the Investigator's clinical judgment or based on the C-SSRS, corresponding to a response of “Yes” on Item 4 (active suicidal ideation with some intent to act, without specific plan) or Item 5 (active suicidal ideation with specific plan and intent); or a history of suicidal behavior within the past year prior to the start of the screening/prospective observational phase.

Has had major surgery (e.g., requiring general or local anesthesia) within the 4 weeks before screening, or will not have fully recovered from surgery or planned surgery during the time the subject is expected to participate in the study.

Has moderately impaired hepatic function at screening, defined as serum alanine aminotransferase (ALT) or aspartate aminotransferase (AST)>2×upper limit of normal (ULN) or total bilirubin (TBL)>2×ULN.

Has received any disallowed therapies as follows: receipt of a known potent inhibitor of hepatic cytochrome P450 (CYP) 2B6, or CYP3A, activity within 1 week or within a period 5 times the drug's half-life, whichever is longer, before the first administration of study drug on Day 1; treatment with a disallowed antipsychotic within the past 30 days prior to screening, except subjects who are on stable doses of quetiapine, aripiprazole, brexpiprazole, or olanzapine prescribed as adjunct treatment for depression (without psychosis) may be included in the study; any changes in psychotropic medication type or dose within the past 30 days prior to screening; treatment with monoamine oxidase inhibitors (MAOIs) currently or within the past 30 days of screening; doses of oral contraception should not contain more than 30 micrograms of ethinyl estradiol per day.

Has initiated psychotherapy (e.g., Cognitive Behavior Therapy, Interpersonal Psychotherapy, Psychodynamic Psychotherapy other than psychoeducation, or acupuncture within the past 90 days of screening. Patients planning to initiate individual or group therapy during the study are also not eligible.

Has received electroconvulsive therapy, transcranial magnetic stimulation, vagal nerve stimulation, deep brain stimulation, or other brain stimulation treatment within the past 4 weeks or currently used as either an acute or maintenance treatment of depression.

Has received any investigational product (IP) within 30 days or 5 half-lives prior to dosing with R(−)-ketamine.

Has a history of substance abuse (drug or alcohol) or dependence (except nicotine or caffeine) within the previous 6 months prior to the screening visit.

Has a positive urine drug screen for ketamine, opiates, cocaine, barbiturates, and/or amphetamine/methamphetamine or positive alcohol screen at Screening or Day −1.

Subjects who have a positive test result at screening due to prescribed opiates or amphetamines may be permitted to continue the screening phase if the prohibited medication is discontinued at least 1 week or 5 half-lives, whichever is longer, before the first dose of study medication. Retesting is not permitted for positive test result(s) from nonprescription use of drugs of abuse.

Has a history of previous nonresponse to ketamine, R(−)-ketamine or S(+)-ketamine, or has received 8 or more doses of ketamine, R(−)-ketamine or S(+)-ketamine in their lifetime.

Has a previous history of intolerance to ketamine, R(−)-ketamine, or S(+)-ketamine.

History of abuse of ketamine, R(−)-ketamine, S(+)-ketamine, or phencyclidine.

Subjects should not consume grapefruit, grapefruit juice, or Seville orange related products for 72 hours before IP administration and throughout the study.

Has the presence of clinically relevant long-term COVID-19 symptoms. Has current signs or symptoms of COVID-19.

COVID-19 vaccination is allowed as long as the doses are administered ≥30 days before study drug administration; vaccination is not allowed during the course of the study.

### Abbreviations

5D-ASC 5-Dimensional Altered States of Consciousness Rating Scale
AE Adverse event
ALT Alanine aminotransferase
AST Aspartate aminotransferase
BMI Body mass index
BPRS+ Brief Psychiatric Rating Scale-Modified 4 Components
CADSS Clinician-Administered Dissociative States Scale
CFR Code of Federal Regulations
CGI-I Clinical Global Impression-Improvement
CGI-S Clinical Global Impression-Severity
CI Confidence interval
COVID-19 Coronavirus disease 2019
CRF Case report form
CRO Contract Research Organization
C-SSRS Columbia Suicide Severity Rating Scale
CYP Cytochrome P450
DBP Diastolic blood pressure
DSM-V Diagnostic and Statistical Manual of Mental Disorders, Fifth Edition
ECG Electrocardiogram
EDC Electronic data capture
EoI End-of-infusion
EQ-5D-3 L European Quality-5 Dimensions-3 Levels
EU European Union
EudraCT European Union Drug Regulating Authorities Clinical Trials (database)
FAS Full Analysis Set
GAD-7 Generalized Anxiety Disorder 7-Item Scale
GCP Good Clinical Practice
GGT Gamma-glutamyl transferase
HAM-D Hamilton Depression Rating Scale
HCV Hepatitis C virus
HIV Human immunodeficiency virus
ICE Intercurrent Event
ICF Informed Consent Form
ICH International Council for Harmonisation
IEC Independent Ethics Committee
IMP Investigational medicinal product
IRB Independent Review Board
IUPAC International Union of Pure and Applied Chemistry
IV Intravenous
IWRS Interactive Web Response System
LBBB Left bundle branch block
LS Least squares
LDH Lactate dehydrogenase
MADRS Montgomery Åsberg Depression Rating Scale
MAOI Monoamine oxidase inhibitor
MAR Missing-at-random
MDD Major depressive disorder
MedDRA Medical Dictionary for Regulatory Activities, Version 23.0 or higher
MGH-ATRQ Massachusetts General Hospital Antidepressant Response Questionnaire
MINI Mini-International Neuropsychiatric Interview
MMRM Mixed effects model for repeated measure
MOAA/S Modified Observer's Assessment of Alertness/Sedation n Number of subjects
NMDA N-methyl-D-aspartate
NMDAR N-methyl-D-aspartate receptor
NPL Non-Patent Literature
OCBP Of childbearing potential
PAC Premature atrial contractions
PK Pharmacokinetic(s)
PTL Patent Literature
PVC Premature ventricular contractions
QIDS-SR-14 Quick Inventory of Depressive Symptomatology-14 Items
QIDS-SR-16 Quick Inventory of Depressive Symptomatology-16 Items
QTc Corrected QT interval
QTcF QT interval corrected according to Fridericia's formula
RBBB Right bundle branch block
SAE Serious adverse event
SAP Statistical Analysis Plan
SBP Systolic blood pressure
SD Standard deviation
SpO2 Oxygen saturation
TBL Total bilirubin
TEAE Treatment-emergent adverse event
TRD Treatment-resistant depression
ULN Upper limit of normal
US United States
VAS Visual analog scale

### C. Schedule of Activities

Activities of the study included Visit 1 for screening between days −15 to −2; Visit 2 for baseline assessments on day −1, in-clinic dosing on day 1, and discharge on day 2; Visit 3 for follow-up on day 8 (+1 day); and Visit 4 for follow-up on day 15 (+1 day) ª, as shown in Tables 2, 3 and 4.

TABLE 2

| | | | Visit 2 | | | | | | | Visit 2 | Visit | Visit |
|---|---|---|---|---|---|---|---|---|---|---|---|---|
| | | | Day 1 | | | | | | | Day 2 | 3 | 4 |
| | | | After Start of Infusion^{b} | | | | | | | | | |
| | | | Pre- | 15 | 40 min | 60 | 2 | 4 | 6 | 24 | 7 | 14 |
| | | | dose | min | (EoI) | min | hr | hr | hr | hr | days | days |
| | Visit | Visit | Notes | | | | | | | | | |
| Procedure | 1 | 2 | c | c | b, c, d | c | c | c | c | c | c | c |
| Informed consent | X | | | | | | | | | | | |
| Inclusion/exclusion | X^{e} | X^{e} | | | | | | | | | | |
| criteria | | | | | | | | | | | | |
| Demography | X | | | | | | | | | | | |
| Physical examination | X | X | | | | | | | | X | | X |
| Height, weight, and | X | | | | | | | | | | | |
| BMI | | | | | | | | | | | | |
| Medical history | X | | | | | | | | | | | |
| Prior/concomitant | X | X | X | z | | | | | | X | X | X |
| medications | | | | | | | | | | | | |
| Serum pregnancy test | X | | | | | | | | | | | |
| (subject OCBP) | | | | | | | | | | | | |
| Urine pregnancy test | | X | | | | | | | | | X | X |
| (subject OCBP) | | | | | | | | | | | | |
| HIV, hepatitis B and C | X | | | | | | | | | | | |
| test | | | | | | | | | | | | |
| Laboratory assessments | X | X | | | | | | | | | X | X |
| 12-Lead ECG^{g} | X | X | X | | X | z | X | X | X | | X | X |
| Vital signs^{h} | X | X | X | | X | | X | X | X | X | X | X |

| | | | | | | |
|---|---|---|---|---|---|---|
| Pulse oximetry (SpO₂) | | | Continuous from pre-dose | | | |
| | | | to 4 hour post-dose | | | |
| Urine drug/alcohol | X | X | | | | |
| screenⁱ | | | | | | |
| Ketamine screen | X | X | | | | |
| AE review | X | X | X | X | X | X |

TABLE 3

| | | | Visit 2 | | | | | | | Visit 2 | Visit | Visit |
|---|---|---|---|---|---|---|---|---|---|---|---|---|
| | | | Day 1 | | | | | | | Day 2 | 3 | 4 |
| | | | After Start of Infusion^{b} | | | | | | | | | |
| | | | Pre- | 15 | 40 min | 60 | 2 | 4 | 6 | 24 | 7 | 14 |
| | | | dose | min | (EoI) | min | hr | hr | hr | hr | days | days |
| | Visit | Visit | Notes | | | | | | | | | |
| Procedure | 1 | 2 | c | c | b, c, d | c | c | c | c | c | c | c |

Scales and Questionnaires

| MINI | X | | | | | | | | | | |
|---|---|---|---|---|---|---|---|---|---|---|---|
| MGH-ATRQ | X | | | | | | | | | | |
| HAM-Dⁱ | X | X | X | z | | | | | X | X | X |
| MADRS^{j, k} | X | | X | | | X | X | z | X | X | X |
| QIDS-SR-16 | | | X | | | | | | X^{l} | X | X |
| CGI-Sⁱ | | | X^{m} | | | | | | X | X | X |
| CGI-I^{j} | | | | | | | | | X | X | X |
| GAD-7^{j} | | | X | | | | | | X | X | X |
| EQ-5D-3L^{j} | | | X | | | | | | X | X | X |
| MOAA/S | | | X | Every 20 min | | | X | X | | | |
| | | | | for first 2 hr | | | | | | | |
| CADSS | | | X | | X | X | X | | | | |
| BPRS+ | | | X | | X | X | X | | | | |
| 5D-ASC | | | | | | | | X | | | |
| C-SSRSⁿ | X | X | | | | | | | X | X | X |

TABLE 4

| | | Visit 2 Day 1 | | | | | | |
|---|---|---|---|---|---|---|---|---|
| | | After Start of Infusion^{b} | | | | | | |
| | Visit | Pre- | 15 | 40 min | 60 | 2 | 4 | 6 |
| | 2 | dose | min | (EoI) | min | hr | hr | hr |
| | Day | Notes | | | | | | |
| Procedure | −1 | c | c | b, c, d | c | c | c | c |

| Study | | | |
|---|---|---|---|
| Administration | | | |
| Admission | X | | |
| Randomization | | X | z |
| Treatment | | | 40 min |
| Administration | | | |

### Notes Applicable to Tables 2, 3 and 4.

a. Subjects who discontinue the study early will complete Visit 4 assessments and procedures as early termination procedures.
b. All post-dose timepoints are relative to the start of infusion except for the 40-minute (EoI) timepoint. EoI procedures should occur 40 minutes after the start of infusion or at the end of infusion if the infusion continues beyond 40 minutes.
c. Where activities at a given timepoint coincide, consideration should be given to ensure that the following order of activities is maintained: Efficacy rating scales (where more than one efficacy scale is assessed at a timepoint, the order of assessments should be MADRS, CGI-I and CGI-S, QIDS-SR-14/16, HAM-D, GAD-7 and EQ5D-3 L); 12-lead ECGs; vital signs and pulse oximetry; safety rating scales (order: MOAA/S, CADSS and BPRS+); AE review, concomitant medication review, physical exam, and clinical laboratory assessment (including urine pregnancy test and drug/alcohol screen).
d. Assessments and procedures should be started as close as possible after the EoI.
e. Check eligibility.
f. Assess clinical hematology and chemistry tests (non-fasting blood samples will be requested, but both fasting and non-fasting samples are acceptable).
g. To be collected prior to clinical laboratory assessments. An ECG will be performed after 5 minutes in a supine position, if possible.
h. To be collected prior to clinical laboratory assessments. Vital signs will include SBP/DBP, heart rate, respiratory rate, and temperature (tympanic/temporal). Blood pressure and pulse measurements should be taken after the subject has been in a supine or semi-supine position in a rested and calm state for at least 5 minutes. Blood pressure must be recorded using the same arm and position (supine or semi-supine) throughout the study, where possible. Three consecutive blood pressure readings will be recorded at intervals of at least 1 minute and the average of the 3 blood pressure readings will be recorded in the CRF. An abnormal blood pressure value at screening may be repeated once after 10-15 minutes of relaxation to determine the subject's eligibility.
i. Urine drug screen will be conducted by a local laboratory and include alcohol, ketamine, opiates, cocaine, barbiturates, and/or amphetamine/methamphetamine. Benzodiazepines are allowed up to 6 hours before the start of the infusion, and if needed as rescue medication.
j. The HAM-D, MADRS, CGI-S, CGI-I, GAD-7, and EQ-5D-3 L should be evaluated by a separate Investigator who is not involved with the assessment of safety to avoid functional unblinding.
k. There are 3 versions of MADRS: 7-day recall, 24-hour recall, and 2-hour recall. For the 2 hour and 4 hour periods, the sleep and appetite items will not be assessed; pre-dose scores for these items obtained on the same day will be carried forward.
l. The QIDS-SR-14, with a 24 hour recall period, will be used on Visit 2 Day 2 assessment due to recall period.
m. Note: The pre-dose CGI-S will be used as the basis for calculating the CGI-I at Visit 2.
n. Note: C-SSRS may be evaluated by either efficacy or safety rater.

### Study Treatment Compliance

Any departures from the prescribed dosage, timing, and mode of administration was recorded in the appropriate CRFs. Noncompliance is defined as receiving less than the full infusion or a deviation from the 40-minute infusion time. Any medication or vaccine (including over-the-counter or prescription medicines, vitamins, and/or herbal supplements) that the subject received at the time of enrollment (within 30 days before the screening visit) or during the study was recorded on the CRF. The following medications were prohibited for the duration of the study beginning from 1 week (or 5 half-lives, whichever is longer) prior to Baseline (Day-1). Receipt of a known potent inhibitor of hepatic cytochrome P450 (CYP) 2B6, or CYP3A, activity within 1 week or within a period 5 times the drug's half-life, whichever was longer, before the first administration of study drug on Day 1. Treatment with a disallowed antipsychotic within the past 30 days prior to screening, except subjects who were on stable doses of quetiapine, aripiprazole, brexpiprazole, or olanzapine prescribed as adjunct treatment for depression (without psychosis) may be included in the study. Any changes in psychotropic medication type or dose within the past 30 days prior to screening. Treatment with monoamine oxidase inhibitors (MAOIs) currently or within the past 30 days of screening. The following medications were restricted for the duration of the study beginning from the date of the screening visit until the completion of assessments at Visit 4 (Day 15) or the discontinuation visit: daily doses of benzodiazepine receptor agonists will be limited to 2 mg/day or less of lorazepam or equivalents. Benzodiazepines were prohibited within 6 hours prior to dosing. Short acting benzodiazepines except clobazam may be used as rescue medication to treat intolerable hallucinations/dissociative effects or any other symptoms in the judgement of the Investigator. Rescue medications may be used for other symptoms including anxiety/agitation, nausea/vomiting, dissociation, and blood pressure/hypertension according to the judgement of the Investigator. Doses of oral contraception should not contain more than 30 micrograms of ethinyl estradiol per day. If applicable, the possible effects of these medications on the efficacy endpoints will be considered during the assessment of the evaluable period. IMP dose modification is not permitted during the study.

### D. Study Assessments and Procedures

The Investigator or his/her qualified designee completed the sponsor specified training for each rating scale (where appropriate), before any rating scales were completed, and some additional training certification, if required, prior to site activation. To maintain the study blind, the staff member that completed the efficacy scales did not complete any safety scales, except for C-SSRS, or assessment of adverse events for any given subject. Similarly, the staff member that completed the safety scales did not complete any efficacy scales for any given subject. To ensure consistency, the same staff member (where possible) completed the questionnaires for each subject throughout the study. All post-dose timepoints in the Schedule of Activities (Section C) were relative to the start of the infusion, except for the 40-minute (end-of-infusion [EoI]) timepoint. EoI procedures which occurred 40 minutes after the start of infusion or at the end of infusion if the infusion continued beyond 40 minutes. EoI procedures included 12-lead ECG, vital signs, pulse oximetry, CADSS, and BPRS+. MOAA/S continued on 20 minute intervals from the start-of-infusion and AE review was continuous from pre-dose to 4 hours post-dose. For the EoI, a time window of ±2 minutes after the 40-minute total time was allowed. Assessments and procedures, however, were started as close as possible after EoI. Study procedures and their timing is summarized in the Schedule of Activities.

Protocol waivers or exemptions were not allowed. Adherence to the study design requirements, including those specified in the Schedule of Activities, was essential and required for study conduct. All screening evaluations were completed and reviewed to confirm that potential subjects met all eligibility criteria. The Investigator maintained a screening log to record details of all subjects screened and to confirm eligibility or record reasons for screening failure, as applicable. Procedures conducted as part of the subject's routine clinical management (e.g., blood count) and obtained before signing of the ICF may be utilized for screening or baseline purposes provided the procedures met the protocol-specified criteria and were performed within the time frame defined in the Schedule of Activities.

Screening and/or baseline procedures included: obtaining informed consent; confirming that the subject met all eligibility criteria; recording the subject's demographic information; performing a physical exam (include recording of the subject's height, weight, and BMI); recording the subject's medical history; conducting screening tests for HIV, hepatitis B, and hepatitis C; recording the subject's prior and concomitant medications; for subjects OCBP, performing a serum pregnancy test at screening and a urine pregnancy test at baseline; conducting laboratory assessments; conducting a 12-lead ECG, vital signs, pulse oximetry; conducting a urine alcohol and drug screen (at a minimum including ketamine, opiates, cocaine, barbiturates, and/or amphetamine/methamphetamine); reviewing AEs; performing an MGH-ATRQ, HAM-D, MADRS, and C-SSRS; and performing a MINI version 7.0.2 to assess the 17 most common psychiatric disorders in DSM-III-R, DSM-IV, DSM-V, and ICD-10. The MINI is designed as a brief structured diagnostic interview to meet the need for a short but accurate structured psychiatric interview for multicenter clinical trials and epidemiology studies and to be used as a first step in outcome tracking in non-research clinical settings. The MINI is a structured interview in which patients are asked to answer questions “Yes” or “No” (e.g., “Were you ever depressed or down, or felt sad, empty or hopeless most of the day, nearly every day, for two weeks?”). The MINI is designed to map onto diagnoses defined by the DSM-5 (Non-Patent Literature 37).

### D.1 Efficacy Assessments

Efficacy assessments were conducted at the time points listed in the Schedule of Activities in Section C.

D.1.1 Montgomery Åsberg Depression Rating Scale

The 10-item MADRS was administered by the study staff to measure the overall severity of depressive symptoms. The MADRS total score was assessed at screening and baseline (Day 1 pre-dose). The primary efficacy endpoint of improvement in the MADRS total score was evaluated prior to discharge on Day 2 of Visit 2, 24 hours after the start of the infusion of study treatment. The MADRS total score was also assessed at 2 hours, 4 hours, 7 days (on Day 8), and 14 days (on Day 15) after the start of the infusion to evaluate the secondary efficacy endpoints of response (defined as ≥50% improvement in the MADRS total score from pre-dose) and remission (defined as MADRS total score≤10) in the improvement of depressive symptoms in the subjects. While the MADRS is typically used with a 7-day recall period, versions of the MADRS including 2-hour and 24-hour recall periods were used in this study. (For the 2 hour and 4 hour periods, the sleep and appetite items were be assessed; pre-dose scores for these items obtained on the same day were carried forward).

### D.1.2 Hamilton Depression Rating Scale

The Hamilton Depression Rating Scale is a 17-item questionnaire designed to assess a subject's level of depression before, during, and after treatment. The survey is used as a guide to evaluate recovery and is based on the clinician's interview with the subject. The HAM-D rates the severity of depression by probing mood, feelings of guilt, suicide ideation, insomnia, agitation or retardation, anxiety, weight loss, and somatic symptoms. Scoring take about 15 minutes. The rater enters a number for each symptom that ranges from 0 (not present) to 4 (extreme symptoms). During this study, the questionnaire was completed at screening, baseline, predose on Day 1, at Day 2 discharge, and both follow-up visits.

### D.1.3 Quick Inventory of Depressive Symptomatology—Self Report

Subjects answered the questions in the QIDS-SR-16, an instrument that is sensitive in detecting changes in the symptoms of depression. The 16 items correlate with the 9 DSM-V symptom criterion domains that include: sleep disturbance-initial, middle, and late insomnia or hypersomnia (Questions 1 to 4); sad mood (Question 5); decrease/increase in appetite/weight (Questions 6 to 9); concentration (Question 10); self-criticism (Question 11); suicidal ideation (Question 12); interest (Question 13); energy/fatigue (Question 14); and psychomotor agitation/retardation (Questions 15 and 16). While the QIDS-SR-16 is typically used with a 7-day recall period, the QIDS-SR-14, with a 24-hour recall period was used on Day 2. For the 24 hour recall periods, the weight and appetite items were assessed; pre-dose scores for these items obtained on Day 1 pre-dose were carried forward. The QIDS-SR-16 item version was used on Day 1 pre-dose and Day 8 and Day 15 follow-up.

### D.1.4 Generalized Anxiety Disorder 7-Item Scale

The GAD-7 is a self-reported questionnaire for the screening and measurement of the severity of generalized anxiety disorder. It has sensitivity and specificity as a screener for panic, social anxiety, and posttraumatic stress disorder. The GAD-7 is comprised of 7 items that measure the severity of various signs of GAD according to reported response categories with assigned points. The scores for all 7 items are added together to obtain the total score on which the assessment of anxiety is made.

### D.1.5 Clinical Global Impression

The CGI rating scales measure symptom severity, treatment response, and the efficacy of treatments in studies of patients with mental disorders. The questionnaires require the study staff to compare the subjects to typical patients in the clinician's experience. The CGI-S assesses minimal clinically important differences in treatments with respect to the severity of depression symptoms. The CGI-I assesses minimal clinically important differences in treatments with respect to the improvement in depression symptoms.

### D.1.6 European Quality—5 Dimensions—3 Levels

EQ-5D-3 L (a questionnaire to assess the health-related quality of life for the domains of mobility, capacity for self-care, conduct of usual activities, pain/discomfort, and anxiety/depression). The EQ-5D-3 L is a questionnaire that essentially consists of 2 pages—the EQ-5D descriptive system and a visual analog scale (VAS). The descriptive system is comprised of 5 dimensions: mobility, self-care, usual activities, pain/discomfort, and anxiety/depression. Each dimension has 3 levels: no problems, some problems, and extreme problems. The subject will be asked to indicate his/her health state by marking the box for the most appropriate statement in each of the 5 dimensions. The VAS records the subject's self-rated health on a vertical VAS where the endpoints are labeled “best imaginable health state” and “worst imaginable health state.” This information was used as a quantitative measure of health outcome.

### D.2 Safety Assessments

The planned time points for all safety assessments are provided in the Schedule of Activities in Section C.

### D.2.1 Physical Examinations

A complete physical examination included, at a minimum, assessments of the cardiovascular, respiratory, gastrointestinal, and neurological systems. Height and weight were also measured and recorded and BMI will be calculated and recorded at screening.

### D.2.2 Vital Signs

Tympanic or temporal temperature, heart rate, blood pressure (SBP/DBP), respiratory rate, and SpO2 were assessed. Blood pressure and pulse measurements will be assessed with a completely automated device while the subject is supine or semi-supine. Manual techniques will be used only if an automated device is not available. Blood pressure and pulse measurements should be preceded by at least 5 minutes of rest for the subject in a quiet setting without distractions (e.g., television, cell phones). An abnormal blood pressure value at screening may be repeated once after 5 minutes of relaxation to determine the subject's eligibility. Blood pressure was recorded using the same arm and position (supine or semi-supine) throughout the study, where possible. Vital signs (to be taken before blood collection for laboratory tests) consisted of 1 pulse and 3 blood pressure measurements (3 consecutive blood pressure readings will be recorded at intervals of at least 1 minute). The average of the 3 blood pressure readings was recorded in the CRF. A pulse oximeter was used to measure peripheral capillary oxygen saturation (SpO2) continuously from pre-dose until 4-hours post-dose. A pulse oximeter was clipped onto the finger or foot of the subject, and light will be sent through the finger or foot and measured on the other side.

### D.2.3 Concomitant Medication Review

Concomitant medications were recorded at the timepoints indicated in Section C and throughout the study until the second follow up visit.

### D.2.4 Electrocardiograms

12-Lead ECGs were obtained as outlined in the Schedule of Activities (see Section C) using an ECG machine that automatically calculates the heart rate and measures PR, QRS, QT, and corrected QT (QTc) intervals. If abnormal, the ECG was repeated. All ECGs were centrally reviewed by an independent cardiologist blinded to treatment allocation; however, central review of the baseline (Day −1) ECG was not required prior to dosing on Day 1.

### D.2.5 Clinical Safety Laboratory Assessments

See Section H for the list of clinical laboratory tests performed and to the Schedule of Activities (Section C) for the timing and frequency. The Investigator reviewed the laboratory report, documented this review, and recorded any clinically relevant changes occurring during the study in the AE section of the CRF. The laboratory reports were filed with the source documents. Clinically significant abnormal laboratory findings are those that are not associated with the underlying disease, unless judged by the Investigator to be more severe than expected for the subject's condition. All laboratory tests with values considered clinically significantly abnormal during participation in the study or within 15 days after the last dose of study treatment were repeated until the values return to normal or baseline or were no longer considered clinically significant by the Investigator or Medical Monitor. If such values did not return to normal/baseline within a period of time judged reasonable by the Investigator, the etiology was identified, and the Sponsor notified. All protocol-required laboratory assessments, as defined in Section H, were conducted in accordance with the laboratory manual and the Schedule of Activities. If laboratory values from non-protocol specified laboratory assessments performed at the institution's local laboratory required a change in subject management or were considered clinically significant by the Investigator (e.g., SAE, AE, or dose modification), then the results were recorded in the CRF.

### D.2.6 Modified Observer's Assessment of Alertness Sedation

The MOAA/S will were to determine if R(−)-ketamine causes the side effect of sedation and to what degree. Sedation was evaluated by the study staff using the MOAA/S. The observer recorded the subject's alertness pre-dose, every 20 minutes for first 2 hours post-dose, and at 4 and 6 hours after the start of the infusion.

### D.2.7 Clinician-Administered Dissociative States Scale

The CADSS was to assess the alertness or dissociative state of subjects administered R(−)-ketamine. The study staff evaluated the subject's degree of dissociation by administering the CADSS questionnaire pre-dose; at 40 minutes post-dose (at EoI); and at 2 and 4 hours post-dose.

### D.2.8 Brief Psychiatric Rating Scale—Modified 4 Components

The BPRS+ is one of the oldest, most widely used rating scales to measure psychotic symptoms and was used by the study staff to measure psychiatric symptoms such as depression, anxiety, hallucinations, and unusual behavior. Only the four-item positive symptom subscale (consisting of: suspiciousness, hallucinations, unusual thought content, and conceptual disorganization) was used in the study to assess treatment emergent psychotic symptoms. The questionnaire was administered pre-dose; at 40 minutes post-dose (at EoI); and at 2 and 4 hours post-dose.

### D.2.9 5-Dimensional Altered States of Consciousness Rating Scale

The 5D-ASC wase used to assess the quality of any acute psychological effects of the R(−)-ketamine infusion. It is a retrospectively assessed questionnaire to measure subjective experiences of altered states of consciousness and contains 94 items that are formulated as a visual analog scale. The test was performed 6 hours after the start of the infusion of R(−)-ketamine.

### D.2.10 Suicidal Risk Monitoring

R(−)-ketamine is being investigated as an antidepressant/central nervous system-active study treatment. Although there has been some concern that these types of treatments may be associated with an increased risk of suicidal ideation or behavior when given to some subjects with MDD, other studies have indicated that ketamine may be associated with a decrease in suicidal ideation or behavior. Therefore, the Sponsor considers it important to monitor suicidal ideation and behavior and treatment-emergent suicidal ideation and behavior during the study. The definitions of behavioral suicidal events used in this scale are based on those used in the Columbia-Suicide History Form. Questions are asked on suicidal behavior, suicidal ideation, and intensity of ideation. At screening (Visit 1), questions will be in relation to lifetime experiences. Questioning at all subsequent visits (see Section C, Schedule of Activities) will be in relation to the last assessment (since last visit). The adult C-SSRS was used for all subjects. The C-SSRS was administered to subjects at screening, at baseline, and prior to discharge from the clinic 24 hours after the infusion of study treatment. The C-SSRS was administered to subjects at follow-up Visit 3 (Day 8) and Visit 4 (Day 15). The C-SSRS was evaluated by either efficacy or safety rater.

### D.3 Adverse Events

The definitions of an AE and SAE can be found in Section F. AEs were reported by the subject (or, when appropriate, by a caregiver, surrogate, or the subject's legally authorized representative). The Investigator and any designees were responsible for detecting, documenting, and recording events that meet the definition of an AE or SAE and remained responsible for following up AEs that were serious, considered related to the study treatment or study procedures, or that caused the subject to discontinue from the study.

### D.3.1 Time Period and Frequency for Collecting Adverse Event and Serious Adverse Event Information

All AEs were collected from the signing of the ICF until the second follow-up visit (Day 15) at the time points specified in the Schedule of Activities (Section C). Medical occurrences that began before the start of study treatment but after obtaining informed consent were recorded on the Medical History/Current Medical Conditions section of the CRF, not the AE section. All SAEs were recorded and reported to the Sponsor or designee within 24 hours as indicated in Section F. The Investigator submitted any updated SAE data to the Sponsor or designee within 24 hours of it being available. Investigators were not obligated to actively seek AE or SAE information after the conclusion of study participation. However, if the Investigator learns of any SAE, including a death, at any time after a subject has been discharged from the study, and he/she considers the event to be reasonably related to the study treatment or study participation, the Investigator must promptly notify the Sponsor or designee. The method of recording, evaluating, and assessing the causality of AEs and SAEs and the procedures for completing and transmitting SAE reports are provided in Section F.

### D.3.2 Method of Detecting AEs and SAEs

Care was taken not to introduce bias when detecting AEs and/or SAEs. Open-ended and non-leading verbal questioning of the subject was the preferred method to inquire about AE occurrences.

### D.3.3 Follow-Up of Adverse Events and Serious Adverse Events

After the initial AE/SAE report, the Investigator was required to proactively follow each subject at subsequent visits/contacts. All SAEs were followed until resolution, stabilization, the event was otherwise explained, or the subject was lost to follow-up. Further information on follow-up procedures is given in Section F.

### D.3.4 Regulatory Reporting Requirements for Serious Adverse Events

Prompt notification by the Investigator to the Sponsor or designee of an SAE was essential so that legal obligations and ethical responsibilities toward the safety of subjects and the safety of a study treatment under clinical investigation are met. An Investigator who receives an Investigator safety report describing an SAE or other specific safety information (e.g., summary or listing of SAEs) from the Sponsor will review and then file it along with the Investigator's Brochure and will notify the IEC/IRB, if appropriate according to local requirements.

### D.3.5 Pregnancy

Details of all pregnancies in female subjects and, if indicated, female partners of male subjects were collected after the start of study treatment and until the second follow-up visit. If a pregnancy was reported, the Investigator informed the Sponsor within 24 hours of learning of the pregnancy. Information on the status of the mother and child was forwarded to the Sponsor. Generally, the follow-up will be no longer than 6 to 8 weeks following the estimated delivery date. Any termination of the pregnancy will be reported regardless of fetal status (presence or absence of anomalies) or indication for the procedure. Abnormal pregnancy outcomes (e.g., spontaneous abortion, fetal death, stillbirth, congenital anomalies, ectopic pregnancy) are considered SAEs.

### D.3.6 COVID-19

If a subject tests positive for COVID-19, this was recorded as either an AE of “symptomatic COVID-19 disease” or “asymptomatic COVID-19 disease.” Symptoms, signs, and sequelae of COVID-19 were reported as an AE per the Definitions and Procedures for Recording, Evaluating, Follow-up, and Reporting (Section F). Medications for the prevention or treatment of COVID-19 were entered as concomitant medications. (Please refer to the EDC Completion Guidelines for further details.)

### D.4 Treatment of Overdose

An overdose was unlikely during this study since all treatment will be administered in a clinic setting using an infusion pump. However, if the study treatment was administered in less than 40 minutes due to a pump error, the Investigator did the following: contact the Medical Monitor immediately; closely monitor the subject for any AE/SAE and laboratory abnormalities for at least 24 hours; obtain a plasma sample for pharmacokinetic (PK) analysis as soon as possible and note the time from the start of the infusion of study treatment if requested by the Medical Monitor (determined on a case-by-case basis); and document the quantity of the excess dose as well as the duration of the overdose in the CRF.

### E. Statistical Considerations

The intent of the primary efficacy analysis was to demonstrate superiority of at least 1 therapeutic dose of R(−)-ketamine solution for injection (30 mg or 60 mg) versus placebo based on the change in the MADRS total score from pre-dose to 24 hours post-dose. For the primary analysis, a sample size of 101 randomized subjects (33:35:33) provided 80% power to detect an 8-point difference between each R(−)-ketamine solution for injection dose and placebo in the mean change from baseline MADRS total score at 24 hours post-dose, using a t-test with a =0.05 (2-tailed) and assuming a common SD of 11.

All statistical analyses, including summary tables and data listings were performed using SAS® software (version 9.4 or higher). Continuous endpoints were summarized using descriptive statistics (number of subjects [n], mean, standard deviation [SD], median, minimum and maximum). Categorical endpoints were summarized using frequency counts and percentages. All individual subject data were presented in listings.

The Full Analysis Set (FAS) consisted of all randomized subjects who received at least one dose of study treatment and had at least one postbaseline assessment available. This population served as the basis for efficacy analysis. Subjects were analyzed according to their randomized treatment. The Safety Analysis Set consisted of all randomized subjects who received any study treatment, even a partial dose. This population was used for all summaries of subject disposition, demographic and baseline data, and safety information including AE incidence. Subjects were analyzed according to the treatment they actually received.

### Primary Endpoint

The change from baseline in MADRS scores was summarized separately by treatment arm for each visit. Associated baseline scores were taken as the last corresponding measurement prior to the first dose of IMP in the treatment period (i.e., Visit 2, pre-dose). The primary endpoint of change from baseline MADRS total score at 24 hours post start of infusion was evaluated using a mixed effects model for repeated measure (MMRM) analysis with observed cases only. The MMRM model includes fixed effects for treatment group, region, visit, study site and treatment group by visit interaction, with subject as a random effect and baseline score as a covariate. An unstructured covariance matrix was used to estimate the variance-covariance structure within subjects across time points. If convergence was not obtained, then other covariance structures to be used will be specified in the SAP. Objective criteria for assessing normality assumptions and proposed alternative analyses were specified in the SAP. From this analysis the least squares (LS) mean estimates for each treatment arm at each visit, along with the standard error and 95% confidence intervals (CIs) were presented separately. In addition, estimates of the treatment difference at each visit were presented along with standard errors of the difference and 95% CIs. The primary comparison for the MADRS is the estimate of the treatment difference at 24 hours after the start of the infusion.

### Secondary Endpoints

The change from baseline to Day 14 in MADRS, HAM-D, GAD-7 and QIDS-SR scores were analyzed using similar model approaches as for MADRS. Observed and change from baseline values in continuous secondary endpoints were summarized descriptively by treatment group. Continuous secondary efficacy endpoints assessed at more than one post-baseline visit were analyzed using an MMRM analysis. The MMRM model includes fixed effects for treatment group, region, visit, study site and treatment group by visit interaction, with subject as a random effect and baseline score as a covariate. From this analysis the LS mean estimates for each treatment arm at each visit, along with the standard error and 95% CIs was presented separately. In addition, estimates of the treatment difference at each visit were presented along with standard errors of the difference and 95% CIs. The numeric values of CGI-S and CGI-I assessments were analyzed separately using similar model approaches as for MADRS. For CGI-S, baseline scores were included as a covariate. Both values for the original categorical scale and the converted numerical scale at each visit (including change from baseline for the CGI-S numerical scale) were summarized using standard summary statistics. Binary exploratory endpoints were analyzed separately and odds ratios, 95% CI and p-value will be presented. Full details of the efficacy analyses and any further supplementary analyses deemed appropriate are provided in the SAP.

### F. Adverse Events and Serious Adverse Events

### AE Definition

An AE is any untoward medical occurrence in a patient or subject, temporally associated with the use of study treatment, whether or not considered related to the study treatment. An AE can therefore be any unfavorable and unintended sign (including an abnormal laboratory finding), symptom, or disease (new or exacerbated) temporally associated with the use of study treatment.

### Events Meeting the AE Definition

Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the Investigator (i.e., not related to progression of underlying disease).

Exacerbation of a chronic or intermittent pre-existing condition including either an increase in frequency and/or intensity of the condition.

New conditions detected or diagnosed after study treatment administration even though it may have been present before the start of the study.

Signs, symptoms, or the clinical sequelae of a suspected drug-drug interaction.

Signs, symptoms, or the clinical sequelae of a suspected overdose of either study treatment or a concomitant medication. Overdose per se will not be reported as an AE/SAE unless it is an intentional overdose taken with possible suicidal/self-harming intent. Such overdoses should be reported regardless of sequelae.

“Lack of efficacy” or “failure of expected pharmacological action” per se will not be reported as an AE or SAE. Such instances will be captured in the efficacy assessments. However, the signs, symptoms, and/or clinical sequelae resulting from lack of efficacy will be reported as AEs or SAEs if they fulfill the definition of an AE or SAE.

### Events NOT Meeting the AE Definition

Any clinically significant abnormal laboratory findings or other abnormal safety assessments which are associated with the underlying disease, unless judged by the Investigator to be more severe than expected for the subject's condition. The disease/disorder being studied or expected progression, signs, or symptoms of the disease/disorder being studied, unless more severe than expected for the subject's condition. Medical or surgical procedure (e.g., endoscopy, appendectomy): the condition that leads to the procedure is the AE. Situations in which an untoward medical occurrence did not occur (social and/or convenience admission to a hospital, including planned hospitalization for this study). Anticipated day-to-day fluctuations of pre-existing disease(s) or condition(s) present or detected at the start of the study that do not worsen.

### Definition of SAE

If an event is not an AE per the definition above, then it cannot be an SAE even if serious conditions are met (e.g., hospitalization for signs/symptoms of the disease under study). A Serious Adverse Event (SAE) is defined as any untoward medical occurrence that, at any dose: a) results in death; b) is life-threatening; c) requires inpatient hospitalization or prolongation of existing hospitalization; d) results in persistent disability/incapacity; or e) is a congenital anomaly/birth defect. The term “life-threatening” in the definition of “serious” refers to an event in which the subject was at risk of death at the time of the event. It does not refer to an event, which hypothetically might have caused death, if it were more severe. In general, hospitalization signifies that the subject has been detained (usually involving at least an overnight stay) at the hospital or emergency ward for observation and/or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious. Hospitalization for elective treatment of a pre-existing condition that did not worsen from baseline is not considered an AE, nor is planned hospitalization for this study. The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) which may interfere with or prevent everyday life functions but do not constitute a substantial disruption.

### Other Situations Related to SAEs

Medical or scientific judgment should be exercised in deciding whether SAE reporting is appropriate in other situations such as important medical events that may not be immediately life-threatening or result in death or hospitalization but may jeopardize the subject or may require medical or surgical intervention to prevent one of the other outcomes listed in the above definition. These events should usually be considered serious. Examples of such events include invasive or malignant cancers, intensive treatment in an emergency room or at home for allergic bronchospasm, blood dyscrasias, or convulsions that do not result in hospitalization, or development of drug dependency or drug abuse.

### AE and SAE Recording

When an AE/SAE occurs, it is the responsibility of the Investigator to review all documentation (e.g., hospital progress notes, laboratory reports, and diagnostics reports) related to the event. The Investigator will then record all relevant AE/SAE information in the CRF. Each event must be recorded separately. The Investigator will attempt to establish a diagnosis of the event based on signs, symptoms, and/or other clinical information. Whenever possible, the diagnosis (not the individual signs/symptoms) will be documented as the AE/SAE.

### Assessment of Intensity

The Investigator will make an assessment of intensity for each AE and SAE reported during the study and assign it to 1 of the following categories:

Mild: An event that is easily tolerated by the subject, causing minimal discomfort and not interfering with everyday activities.

Moderate: An event that causes sufficient discomfort and interferes with normal everyday activities.

Severe: An event that prevents normal everyday activities. An AE that is assessed as severe should not be confused with a SAE. Severe is a category utilized for rating the intensity of an event; and both AEs and SAEs can be assessed as severe.

An event is defined as ‘serious’ when it meets at least one of the predefined outcomes as described in the definition of an SAE, NOT when it is rated as severe.

### Assessment of Causality

The Investigator is obligated to assess the relationship between study treatment and each occurrence of each AE/SAE. The AE must be characterized as unrelated, unlikely to be related, possibly related, probably related, or definitely related. “Definitely related” suggests that the AE has a timely relationship to administration of study treatment, and there is no apparent potential alternate etiology. “Probably related” conveys that there are facts, evidence, and/or arguments to suggest a causal relationship, rather than a relationship cannot be ruled out. “Possibly related” suggests that the association of the AE with the study treatment is unknown; however, the AE is not reasonably supported by other conditions. “Unlikely to be related” suggests that only a remote connection exists between the study treatment and the AE. Other conditions, including chronic illness, progression or expression of the disease state or reaction to concomitant therapy, appear to explain the reported AE. “Unrelated” is used if there is not a reasonable possibility that the study treatment caused the AE.

The Investigator will use clinical judgment to determine the relationship. Alternative causes, such as underlying disease(s), concomitant therapy, and other risk factors, as well as the temporal relationship of the event to study treatment administration will be considered and investigated. The Investigator will also consult the IB and/or Product Information, for marketed products, in his/her assessment. For each AE/SAE, the Investigator must document in the medical notes that he/she has reviewed the AE/SAE and has provided an assessment of causality. There may be situations in which an SAE has occurred, and the Investigator has minimal information to include in the initial report to Safety Management. However, it is very important that the Investigator always makes an assessment of causality for every event before the initial transmission of the SAE data to Safety Management. The Investigator may change his/her opinion of causality in light of follow-up information and send an SAE follow-up report with the updated causality assessment. The causality assessment is one of the criteria used when determining regulatory reporting requirements.

### Follow-Up of AEs and SAEs

The Investigator is obligated to perform or arrange for the conduct of supplemental measurements and/or evaluations as medically indicated or as requested by Safety Management to elucidate the nature and/or causality of the AE or SAE as fully as possible. This may include additional laboratory tests or investigations, histopathological examinations, or consultation with other health care professionals. If a subject dies during participation in the study or during a recognized follow-up period, the Investigator will provide Safety

Management with a copy of any postmortem findings including histopathology. New or updated information will be recorded in the originally completed CRF. The Investigator will submit any updated SAE data to the Sponsor or designee within 24 hours of receipt of the information.

Adverse events will be coded using the Medical Dictionary for Regulatory Activities

(MedDRA; Version 23.0 or higher). Treatment-emergent adverse events (TEAEs) are AEs with onset or worsening after the start of study treatment. The AE summaries will be primarily based on TEAEs. The number and percentage of subjects with TEAEs will be summarized by treatment group, system organ class, and preferred term for all TEAEs, treatment-related TEAEs, SAEs, serious TEAEs, severe TEAEs, TEAEs of special interest, and all TEAEs leading to study drug discontinuation. All TEAEs will be further summarized by maximum severity and causality. The TEAEs of special interest will be identified in the SAP. All AEs will be presented in a by-subject listing. Serious AEs, severe AEs, and AEs leading to study discontinuation or death will be presented in separate listings.

### G. Permitted and Prohibited Concomitant Medications

A list of permitted and prohibited concomitant medications throughout the study is provided in Table 5. Permitted episodic or continuous use of a concomitant medication is indicated with a “Y” (yes). Prohibited episodic or continuous use of a concomitant medication is indicated with an “N” (no).

TABLE 5

| | Episodic | Continuous | |
|---|---|---|---|
| Drug | Use | Use | Comments |
| Amantadine | N | N | |
| Analgesics (except opiates) | Y | Y | |
| Anti-anginal drugs | N | N | Angina is exclusion |
| Anti-arrhythymics | N | N | Arrhythmia is exclusionary |
| Anticholenergics | N | N | |
| Anticholinesterase inhibitors | N | N | |
| Anticoagulants | N | N | |
| Anticonvulsants | N | N | |
| Antidepressants | N | Y | MAOIs are always excluded |
| Antidiarrheal | Y | N | |
| Anti-emetics | Y | N | |
| Anti-inflammatory | Y | Y | |
| Antipsychotics | N | Y | Stable doses of quetiapine, aripiprazole, |
| | | | olanzapine, and brexpiprazole are permitted |
| | | | for continuous use |
| Antifungals | N | N | No ketoconazole, fluconazole due to |
| | | | CYP3A interaction |
| Artemisinin | | | |
| Aspirin | Y | Y | |
| Benzodiazepines | Y | Y | Except clobazam |
| | | | Daily doses of benzodiazepine receptor |
| | | | agonists will be limited to 2 mg/day or less |
| | | | of lorazepam or equivalents. |
| | | | Benzodiazepines will be prohibited within |
| | | | 6 hours prior to dosing |
| Clarithromycin | N | N | CYP3A interaction |
| Erythromycin | N | N | CYP3A interaction |
| Elagolix | N | N | CYP3A interaction |
| Fish oils | Y | Y | |
| HIV drugs | N | N | HIV excluded |
| Hormones | N | Y | Glucocorticoids excluded due to CYP3A |
| | | | interaction |
| Itraconazole | N | N | CYP3A interaction |
| Ketoconazole | N | N | CYP3A interaction |
| Lithium | N | N | |
| Methyldopa | N | N | |
| Modafinil | N | N | |
| Opiates | N | N | |
| Oritavancin | N | N | CYP3A interaction |
| Pioglitazone | N | N | CYP3A interaction |
| Rifabutin | N | N | CYP3A interaction |
| Rifampin | N | N | CYP interaction |
| St. john's wort | N | N | CYP3A interaction |
| Stimulants | N | N | |
| Steroids (oral) | N | N | |
| Steroids (inhaled, topical, | Y | Y | |
| ophthalmic) | | | |
| Telithromycin | N | N | CYP3A interaction |
| Telotristat | N | N | CYP3A interaction |
| Ticlopidine | N | N | CYP2B6 interaction |
| Troglitazone | N | N | CYP3A interaction |
| Troleandomycin | N | N | CYP3A interaction |
| Tryptophan | N | N | |
| Voriconazole | N | N | CYP3A interaction |

### H. Clinical Laboratory Tests

Pregnancy test: Serum testing was performed at screening and to confirm a positive urine test. Local urine pregnancy testing was performed at both follow-up visits. Hepatitis B and hepatitis C screening: hepatitis B surface antigen and hepatitis C virus (HCV antibody) testing will be required. The tests detailed in Table 6 will be performed by a local laboratory. Urine drug screen may be performed on site. Protocol-specific requirements for inclusion or exclusion of subjects are detailed in Section B herein. Additional tests may be performed at any time during the study as determined necessary by the Investigator or required by local regulations. Investigators documented their review of each laboratory safety report. Laboratory/analyte results that could unblind the study were reported to study centers or other blinded personnel until the study was unblinded.

TABLE 6

| Laboratory | | | | |
|---|---|---|---|---|
| Assessments | Parameters | | | |
| Hematology | Platelet count | RBC indices: | | White blood cell |
| | Red blood cell | Mean corpuscular volume | | count with |
| | (RBC) count | (MCV) Mean corpuscular | | differential: |
| | Hemoglobin | hemoglobin (MCH) | | Neutrophils |
| | Hematocrit | % Reticulocytes | | Lymphocytes |
| | | | | Monocytes |
| | | | | Eosinophils |
| | | | | Basophils |
| Clinical | Blood urea | Potassium | Aspartate aminotransferase | Total and direct |
| chemistry | nitrogen or urea | | (AST)/serum glutamic- | bilirubin |
| | Lactate | | oxaloacetic transaminase | |
| | dehydrogenase | | (SGOT) | |
| | (LDH) | | | |
| | Creatinine | Sodium | Alanine aminotransferase | Total protein |
| | | Chloride | (ALT)/serum glutamic- | |
| | | | pyruvic transaminase | |
| | | | (SGPT) | |
| | Glucose | Calcium | Alkaline phosphatase | |
| | [non-fasting^{a}] | | Gamma-glutamyl | |
| | | | transferase (GGT) | |
| | | | Albumin | |

| | |
|---|---|
| Routine | Specific gravity |
| urinalysis | pH, glucose, protein, blood, ketones, bilirubin, urobilinogen, nitrite, |
| | leukocyte esterase by dipstick |
| | Microscopic examination (if blood or protein is abnormal) |
| Other | Follicle stimulating hormone and estradiol (as needed in women of |
| screening | non-childbearing potential only) |
| tests | Urine alcohol and drug screen (to include at minimum: |
| | amphetamines/methamphetamines, ketamine, barbiturates, cocaine, and |
| | opiates) |
| | Serum or urine human chorionic gonadotropin (hCG) pregnancy test |
| | (as needed for women of childbearing potential)^{b} |
| | Serology (HIV antibody, hepatitis B surface antigen, and HCV antibody) |
| | All study-required laboratory assessments will be performed by a local |
| | laboratory. Urine drug screen may be done on site. The results of each test must |
| | be entered into the CRF. |

^{a}Non-fasting blood samples will be requested, but both fasting and non-fasting samples are acceptable.
^{b}Local urine testing will be standard for the protocol unless serum testing is required by local regulation or IEC/IRB.

### I. Demographics and Baseline Characteristics of Study Subjects

The demographics and baseline characteristics of the study subjects are provided in Table 7, below.

TABLE 7
Demographics

| | | 30 mg | | 60 mg | | Placebo | | Total | |
|---|---|---|---|---|---|---|---|---|---|
| | Statistic | (n = 33) | | (n = 35) | | (n = 33) | | (n = 101) | |
| Age | n | 33 | | 35 | | 33 | | 101 | |
| (years) | Mean | 47.2 | | 43.3 | | 44.3 | | 44.9 | |
| | Median | 50.0 | | 47.0 | | 44.0 | | 47.0 | |
| | SD | 11.36 | | 12.28 | | 11.44 | | 11.71 | |
| | Min, Max | 24, 65 | | 24, 65 | | 22, 64 | | 22, 65 | |
| Age | 18 to <44 years | 15 | (45) | 17 | (49) | 17 | (52) | 49 | (49) |
| category | [n(%)] | | | | | | | | |
| | 45 to 65 years | 18 | (55) | 18 | (51) | 16 | (48) | 52 | (51) |
| | [n(%)] | | | | | | | | |
| Sex | Male [n(%)] | 11 | (33) | 16 | (46) | 13 | (39) | 40 | (40) |
| | Female [n(%)] | 22 | (67) | 19 | (54) | 20 | (61) | 61 | (60) |
| | Not Reported [n(%)] | 0 | (0) | 0 | (0) | 0 | (0) | 0 | (0) |
| Region | United States [n(%)] | 9 | (27) | 10 | (29) | 8 | (24) | 27 | (27) |
| | Europe [n(%)] | 24 | (73) | 25 | (71) | 25 | (76) | 74 | (73) |
| Baseline | n | 33 | | 35 | | 33 | | 101 | |
| MADRS | Mean | 29.5 | | 29.7 | | 29.9 | | 29.7 | |
| total score | Median | 30.0 | | 30.0 | | 30.0 | | 30.0 | |
| | SD | 4.74 | | 4.45 | | 5.44 | | 4.84 | |
| | Min, Max | 20, 41 | | 22, 42 | | 9, 39 | | 9, 42 | |
| Baseline | <35 [n(%)] | 29 | (88) | 31 | (89) | 28 | (85) | 88 | (87) |
| MADRS | >=35 [n(%)] | 4 | (12) | 4 | (11) | 5 | (15) | 13 | (13) |
| total score | | | | | | | | | |
| Baseline | n | 33 | | 35 | | 33 | | 101 | |
| HAM-D | Mean | 24.8 | | 24.1 | | 24.5 | | 24.4 | |
| total score | Median | 24.0 | | 24.0 | | 26.0 | | 25.0 | |
| | SD | 3.06 | | 3.09 | | 4.79 | | 3.70 | |
| | Min, Max | 16, 31 | | 18, 31 | | 12, 33 | | 12, 33 | |
| Body Mass | n | 33 | | 35 | | 33 | | 101 | |
| Index | Mean | 25.2 | | 27.8 | | 26.6 | | 26.5 | |
| (kg/m²) | Median | 25.3 | | 26.9 | | 27.1 | | 26.3 | |
| | SD | 3.50 | | 3.89 | | 3.66 | | 3.81 | |
| | Min, Max | 18, 34 | | 23, 35 | | 19, 33 | | 18, 35 | |
| Duration | n | 33 | | 35 | | 33 | | 101 | |
| of | Mean | 15.6 | | 11.9 | | 13.7 | | 13.7 | |
| depression | Median | 14.0 | | 10.0 | | 12.0 | | 11.0 | |
| (years) | SD | 9.11 | | 7.62 | | 9.55 | | 8.82 | |
| | Min, Max | 3, 36 | | 1, 34 | | 1, 39 | | 1, 39 | |
| Duration | <5 years [n(%)] | 4 | (12) | 2 | (6) | 4 | (12) | 10 | (10) |
| of | 5 to 10 years [n(%)] | 8 | (24) | 17 | (49) | 11 | (33) | 36 | (36) |
| depression | >10 years [n(%)] | 21 | (64) | 16 | (46) | 18 | (55) | 55 | (54) |
| Total | n | 33 | | 35 | | 33 | | 101 | |
| number of | Mean | 5.5 | | 4.9 | | 5.7 | | 5.4 | |
| depressive | Median | 5.0 | | 3.0 | | 4.0 | | 4.0 | |
| episodes to | SD | 3.61 | | 4.14 | | 4.98 | | 4.25 | |
| date | Min, Max | 2, 15 | | 1, 20 | | 2, 25 | | 1, 25 | |
| Duration | n | 33 | | 35 | | 33 | | 101 | |
| of current | Mean | 50.8 | | 53.3 | | 43.8 | | 49.4 | |
| episode | Median | 28.0 | | 31.0 | | 31.0 | | 30.0 | |
| (weeks) | SD | 47.48 | | 48.00 | | 32.79 | | 43.19 | |
| | Min, Max | 20, 242 | | 15, 208 | | 12, 156 | | 12, 242 | |
| Family | Yes¹ [n(%)] | 8 | (24) | 13 | (37) | 12 | (36) | 33 | (33) |
| history of | Alcohol abuse | 0 | (0) | 2 | (6) | 3 | (9) | 5 | (5) |
| psychiatric | Anxiety disorder | 0 | (0) | 0 | 0) | 3 | (9) | 3 | (3) |
| disorder | Bipolar disorder | 2 | (6) | 0 | (0) | 1 | (3) | 3 | (3) |
| | Depression | 7 | (21) | 10 | (29) | 8 | (24) | 25 | (25) |
| | Schizophrenia | 1 | (3) | 1 | (3) | 1 | (3) | 3 | (3) |
| | Substance abuse | 0 | (0) | 0 | (0) | 0 | (0) | 0 | (0) |
| | Other | 0 | (0) | 1 | (3) | 1 | (3) | 2 | (2) |
| | No [n(%)] | 23 | (70) | 20 | (57) | 19 | (58) | 62 | (61) |
| | Unknown [n(%)] | 2 | (6) | 2 | (6) | 2 | (6) | 6 | (6) |
| Number of | <3 [n(%)] | 24 | (73) | 24 | (69) | 22 | (67) | 70 | (69) |
| previous | >=3 [n(%)] | 9 | (27) | 11 | (31) | 11 | (33) | 31 | (31) |
| treatment | | | | | | | | | |
| failures in | | | | | | | | | |
| the current | | | | | | | | | |
| episode | | | | | | | | | |
| Baseline | Selective Serotonin | 12 | (36) | 13 | (37) | 9 | (27) | 34 | (34) |
| medication | reuptake inhibitor | | | | | | | | |
| use¹ | (SSRI) [n(%)] | | | | | | | | |
| | Serotonin and | 23 | (70) | 24 | (69) | 27 | (82) | 74 | (73) |
| | norepinephrine | | | | | | | | |
| | reuptake inhibitor | | | | | | | | |
| | (SNRI) [n(%)] | | | | | | | | |
| | Antidepressant with | 0 | (0) | 2 | (6) | 1 | (3) | 3 | (3) |
| | adjunctive | | | | | | | | |
| | medications [n(%)] | | | | | | | | |
| | None [(n %)] | 0 | (0) | 0 | (0) | 0 | (0) | 0 | (0) |

¹Multiple selections may be reported for each subject.
Note:
HAM-D = Hamilton Depression Rating Scale; MADRS = Montgomery Åsberg Depression Rating Scale.

Table 8 provides a summary of subject disposition at the start of the study, and the number of subjects who terminated early, were lost to follow up, or withdrew.

TABLE 8
Subject Disposition (Screened Analysis Set)

| | 30 mg | | 60 mg | | Placebo | | Total | |
|---|---|---|---|---|---|---|---|---|
| Subjects screened [n] | | | | | | | 132 | |
| Subjects randomized [n] | 33 | | 35 | | 34 | | 102 | |
| Subjects in the full analysis set | 33 | (100) | 35 | (100) | 33 | (97) | 101 | (99) |
| [n(%)] | | | | | | | | |
| Subjects in the safety analysis set | 33 | (100) | 35 | (100) | 34 | (100) | 102 | (100) |
| [n(%)] | | | | | | | | |
| Subjects completed study [n(%)] | 31 | (94) | 35 | (100) | 31 | (91) | 97 | (95) |
| Subjects terminated early from | 2 | (6) | 0 | (0) | 3 | (9) | 5 | (5) |
| the study [n(%)] | | | | | | | | |
| Adverse event | 0 | (0) | 0 | (0) | 1 | (3) | 1 | (1) |
| Death | 0 | (0) | 0 | (0) | 0 | (0) | 0 | (0) |
| Lost to follow-up | 0 | (0) | 0 | (0) | 1 | (3) | 1 | (1) |
| Physician decision | 0 | (0) | 0 | (0) | 0 | (0) | 0 | (0) |
| Pregnancy | 0 | (0) | 0 | (0) | 0 | (0) | 0 | (0) |
| Terminated by Sponsor | 0 | (0) | 0 | (0) | 0 | (0) | 0 | (0) |
| Non-compliance/Protocol | 1 | (3) | 0 | (0) | 0 | (0) | 1 | (1) |
| Violation | | | | | | | | |
| Withdrawal by subject | 1 | (3) | 0 | (0) | 1 | (3) | 2 | (2) |

Percentages are based on the number of subjects randomized.

### H. Results

Results of the study are provided in the tables below.

Tables 9, 10 and 11 show MADRS results, described in Section D.1.1. Analysis methods are described in Section E.

TABLE 9
Summary of Montgomery Åsberg Depression
Rating Scale (MADRS) Total Score.

| | | 30 mg | 60 mg | Placebo |
|---|---|---|---|---|
| | Statistic | (N = 33) | (N = 35) | (N = 33) |
| Baseline¹ | n | 33 | 35 | 32 |
| | Mean | 29.5 | 29.7 | 30.0 |
| | Median | 30.0 | 30.0 | 30.0 |
| | SD | 4.74 | 4.45 | 5.50 |
| | Min, Max | 20, 41 | 22, 42 | 9, 39 |
| 2 hours | n | 33 | 35 | 33 |
| | Mean | 20.7 | 18.4 | 21.6 |
| | Median | 21.0 | 20.0 | 24.0 |
| | SD | 8.59 | 8.34 | 8.56 |
| | Min, Max | 8, 40 | 3, 34 | 4, 37 |
| Change from baseline | n | 33 | 35 | 33 |
| to 2 hours | Mean | −8.8 | −11.3 | −8.3 |
| | Median | −7.0 | −10.0 | −6.0 |
| | SD | 7.82 | 8.35 | 7.15 |
| | Min, Max | −23, 1 | −32, 0 | −24, 3 |
| 4 hours | n | 33 | 35 | 33 |
| | Mean | 19.2 | 17.6 | 19.6 |
| | Median | 19.0 | 18.0 | 21.0 |
| | SD | 8.53 | 8.60 | 7.75 |
| | Min, Max | 6, 39 | 3, 35 | 4, 34 |
| Change from baseline | n | 33 | 35 | 33 |
| to 4 hours | Mean | −10.4 | −12.1 | −10.3 |
| | Median | −10.0 | −12.0 | −11.0 |
| | SD | 8.03 | 8.44 | 7.23 |
| | Min, Max | −26, 1 | −32, 2 | −24, 1 |
| 24 hours (Day 2) | n | 33 | 35 | 33 |
| | Mean | 16.7 | 15.1 | 17.1 |
| | Median | 13.0 | 16.0 | 17.0 |
| | SD | 10.92 | 10.72 | 9.15 |
| | Min, Max | 0, 40 | 0, 35 | 2, 34 |
| Change from baseline | n | 33 | 35 | 33 |
| to 24 hours | Mean | −12.8 | −14.5 | −12.9 |
| | Median | −15.0 | −14.0 | −14.0 |
| | SD | 10.81 | 10.71 | 9.43 |
| | Min, Max | −34, 9 | −42, 3 | −28, 7 |
| Day 8 | n | 30 | 35 | 31 |
| | Mean | 19.9 | 17.7 | 19.4 |
| | Median | 22.0 | 21.0 | 20.0 |
| | SD | 10.00 | 10.51 | 8.91 |
| | Min, Max | 0, 40 | 0, 37 | 1, 35 |
| Change from baseline | n | 30 | 35 | 31 |
| to Day 8 | Mean | −9.8 | −12.0 | −10.8 |
| | Median | −7.0 | −10.0 | −11.0 |
| | SD | 11.53 | 10.62 | 9.65 |
| | Min, Max | −35, 10 | −42, 2 | −29, 4 |
| Day 15 | n | 31 | 35 | 31 |
| | Mean | 22.8 | 19.3 | 20.8 |
| | Median | 24.0 | 22.0 | 21.0 |
| | SD | 9.76 | 11.62 | 8.98 |
| | Min, Max | 2, 43 | 0, 38 | 2, 37 |
| Change from baseline | n | 31 | 35 | 31 |
| to Day 15 | Mean | −6.6 | −10.4 | −9.3 |
| | Median | −5.0 | −6.0 | −9.0 |
| | SD | 10.14 | 11.62 | 9.04 |
| | Min, Max | −31, 14 | −42, 4 | −30, 7 |

For Table 9, the total score is computed as the sum of scores across all 10 items. Each item yields a score of 0 to 6, hence the total score ranges from 0 to 60. Higher score indicates more severe depression.
¹ Baseline value is defined as the last non-missing value prior to study drug administration.

TABLE 10
Primary Analysis of Change from Baseline MADRS Total Score

| | | | | Diff. Adjusted mean (SE) | |
|---|---|---|---|---|---|
| Analysis | | | Adjusted mean | R(−)ketamine - Placebo | |
| timepoint | Treatment | n | (SE) | [95% CI] | p-value |
| Day 1, 2 hours | 30 mg | 33 | −9.7 (1.35) | −0.6 (1.85) [−4.24, 3.08] | 0.7540 |
| | 60 mg | 35 | −12.0 (1.31) | −2.9 (1.82) [−6.55, 0.67] | 0.1096 |
| | Placebo | 33 | −9.1 (1.36) | — | — |
| Day 1, 4 hours | 30 mg | 33 | −11.2 (1.36) | −0.1 (1.86) [−3.81, 3.56] | 0.9462 |
| | 60 mg | 35 | −12.8 (1.32) | −1.7 (1.83) [−5.34, 1.92] | 0.3526 |
| | Placebo | 33 | −11.1 (1.37) | — | — |
| Day 1, 24 hours | 30 mg | 33 | −13.7 (1.75) | −0.0 (2.42) [−4.84, 4.77] | 0.9886 |
| | 60 mg | 35 | −15.3 (1.69) | −1.6 (2.38) [−6.35, 3.11] | 0.4988 |
| | Placebo | 33 | −13.7 (1.76) | — | — |
| Day 8 | 30 mg | 30 | −10.4 (1.81) | 1.0 (2.51) [−4.01, 5.94] | 0.7009 |
| | 60 mg | 35 | −12.7 (1.72) | −1.4 (2.44) [−6.26, 3.45] | 0.5673 |
| | Placebo | 31 | −11.3 (1.81) | — | — |
| Day 15 | 30 mg | 31 | −7.5 (1.82) | 2.4 (2.53) [−2.62, 7.40] | 0.3460 |
| | 60 mg | 35 | −11.1 (1.73) | −1.2 (2.46) [−6.09, 3.69] | 0.6277 |
| | Placebo | 31 | −9.9 (1.83) | — | — |

CI = confidence interval, SE = standard error.

The analysis in Table 10 was performed using a mixed model for repeated measurements (MMRM) with fixed effects for treatment group, analysis visit, region and treatment group-by-visit interaction. The baseline value is included as a covariate. An unstructured covariance matrix is used to model the within-subject variance-covariance errors. The total score is computed as the sum of scores across all 10 items. Each item yields a score of 0 to 6, hence the total score ranges from 0 to 60. Higher score indicates more severe depression.

TABLE 11
Secondary Analysis of Proportion of Subjects with >=50% Improvement of
Montgomery Åsberg Depression Rating Scale (MADRS) Total Score.

| | | | | | Not- | adjusted odds ratio | |
|---|---|---|---|---|---|---|---|
| analysis | | | Responder | | responder | R-ket/placebo | |
| timepoint | treatment | n | [n (%)] | | [n (%)] | (95% CI) | p-value |
| Day 1, 2 | 30 mg | 33 | 9 | (27.3) | 24 (72.7) | 1.667 (0.503, 5.520) | 0.4031 |
| hours | 60 mg | 35 | 11 | (31.4) | 24 (68.6) | 2.031 (0.634, 6.504) | 0.2327 |
| | Placebo | 33 | 6 | (18.2) | 27 (81.8) | — | |
| Day 1, 4 | 30 mg | 33 | 9 | (27.3) | 24 (72.7) | 0.963 (0.317, 2.928) | 0.9472 |
| hours | 60 mg | 35 | 13 | (37.1) | 22 (62.9) | 1.536 (0.534, 4.416) | 0.4259 |
| | Placebo | 33 | 9 | (27.3) | 24 (72.7) | — | — |
| Day 1, | 30 mg | 33 | 19 | (57.6) | 14 (42.4) | 1.418 (0.524, 3.837) | 0.4916 |
| 24 hours | 60 mg | 35 | 16 | (45.7) | 19 (54.3) | 0.847 (0.318, 2.254) | 0.7390 |
| | Placebo | 33 | 16 | (48.5) | 17 (51.5) | — | — |
| Day 8 | 30 mg | 33 | 9 | (30.0) | 21 (70.0) | 1.040 (0.336, 3.212) | 0.9463 |
| | 60 mg | 35 | 12 | (34.3) | 23 (65.7) | 1.202 (0.412, 3.511) | 0.7361 |
| | Placebo | 33 | 9 | (29.0) | 22 (71.0) | — | — |
| Day 15 | 30 mg | 33 | 5 | (16.1) | 26 (83.9) | 0.621 (0.169, 2.282) | 0.4727 |
| | 60 mg | 35 | 12 | (34.3) | 23 (65.7) | 1.715 (0.559, 5.263) | 0.3459 |
| | Placebo | 33 | 7 | (22.6) | 24 (77.4) | — | — |

For Table 11, the total score is computed as the sum of scores across all 10 items. Each item yields a score of 0 to 6, hence the total score ranges from 0 to 60. Higher score indicates more severe depression. Responder status was defined as subjects with at least 50% reduction in

MADRS Total Score at the corresponding timepoint. This analysis was based on a logistic regression using fixed effects of treatment group, analysis visit, region and treatment group-by-visit interaction. The baseline value is included as a covariate. MADRS scores showed a trend towards a positive response to R(−)-ketamine at the higher, 60 mg dose at later time points, with a ˜2 point difference seen at day 15. This difference from placebo was not statistically significant. This observed effect may be low due to the single dose administered, and be greater with repeated dosing.

Tables 13-18 provides a summary of vital signs and pulse oximetry at the indicated time points and R(−)-ketamine dosages. In these tables, baseline value is defined as the last non-missing value prior to study drug administration.

TABLE 13
Systolic blood pressure (mmHg).

| | | 30 mg | 60 mg | Placebo |
|---|---|---|---|---|
| Time point | Statistic | (n = 33) | (n = 35) | (n = 34) |
| Baseline | n | 33 | 35 | 34 |
| | Mean | 118.1 | 118.1 | 119.1 |
| | Median | 120.0 | 118.0 | 121.5 |
| | SD | 9.88 | 10.53 | 9.42 |
| | Min, Max | 94, 140 | 93, 138 | 85, 136 |
| Day 1, 15 minutes | n | 13 | 12 | 16 |
| | Mean | 116.8 | 123.6 | 121.3 |
| | Median | 119.0 | 124.5 | 122.5 |
| | SD | 8.93 | 8.63 | 5.02 |
| | Min, Max | 93, 125 | 111, 143 | 110, 129 |
| Change from Baseline | n | 13 | 12 | 16 |
| to Day 1, 15 minutes | Mean | 1.2 | 4.4 | −0.4 |
| | Median | −1.0 | 1.5 | −1.0 |
| | SD | 5.93 | 9.24 | 4.66 |
| | Min, Max | −7, 14 | −9, 26 | −7, 11 |
| Day 1, 40 minutes | n | 33 | 35 | 33 |
| | Mean | 120.1 | 123.0 | 119.1 |
| | Median | 122.0 | 122.0 | 121.0 |
| | SD | 12.14 | 11.55 | 9.82 |
| | Min, Max | 82, 144 | 100, 148 | 99, 138 |
| Change from Baseline | n | 33 | 35 | 33 |
| to Day 1, 40 minutes | Mean | 2.0 | 4.9 | 0.5 |
| | Median | 0.0 | 3.0 | −2.0 |
| | SD | 9.74 | 8.69 | 8.13 |
| | Min, Max | −18, 25 | −11, 24 | −11, 23 |
| Day 1, 60 minutes | n | 14 | 13 | 17 |
| | Mean | 117.7 | 123.9 | 122.9 |
| | Median | 119.5 | 124.0 | 122.0 |
| | SD | 6.82 | 7.97 | 6.76 |
| | Min, Max | 105, 126 | 110, 138 | 110, 135 |
| Change from Baseline | n | 14 | 13 | 17 |
| to Day 1, 60 minutes | Mean | 2.4 | 5.3 | 1.0 |
| | Median | 2.5 | 5.0 | −1.0 |
| | SD | 7.06 | 8.32 | 6.16 |
| | Min, Max | −8, 14 | −5, 21 | −6, 15 |
| Day 1, 2 hours | n | 33 | 35 | 33 |
| | Mean | 118.7 | 119.0 | 118.7 |
| | Median | 122.0 | 120.0 | 120.0 |
| | SD | 12.39 | 10.40 | 7.76 |
| | Min, Max | 88, 146 | 99, 138 | 98, 134 |
| Change from Baseline | n | 33 | 35 | 33 |
| to Day 1, 2 hours | Mean | 0.6 | 0.9 | 0.1 |
| | Median | 0.0 | 1.0 | 0.0 |
| | SD | 8.25 | 7.30 | 5.49 |
| | Min, Max | −13, 25 | −11, 15 | −12, 13 |
| Day 1, 4 hours | n | 33 | 35 | 33 |
| | Mean | 119.0 | 119.1 | 117.5 |
| | Median | 120.0 | 121.0 | 119.0 |
| | SD | 11.58 | 9.35 | 8.76 |
| | Min, Max | 93, 145 | 102, 135 | 94, 131 |
| Change from Baseline | n | 33 | 35 | 33 |
| to Day 1, 4 hours | Mean | 0.9 | 1.0 | −1.2 |
| | Median | −1.0 | 0.0 | −1.0 |
| | SD | 8.48 | 8.22 | 8.14 |
| | Min, Max | −12, 21 | −15, 23 | −19, 22 |
| Day 1, 6 hours | n | 33 | 34 | 31 |
| | Mean | 118.0 | 119.7 | 118.0 |
| | Median | 120.0 | 119.5 | 121.0 |
| | SD | 8.94 | 9.90 | 8.22 |
| | Min, Max | 100, 132 | 103, 141 | 92, 130 |
| Change from Baseline | n | 33 | 34 | 31 |
| to Day 1, 6 hours | Mean | −0.1 | 1.4 | −0.7 |
| | Median | −2.0 | 2.0 | −1.0 |
| | SD | 6.83 | 7.85 | 6.58 |
| | Min, Max | −12, 14 | −13, 25 | −16, 14 |
| Day 2 - Discharge | n | 33 | 35 | 33 |
| | Mean | 118.0 | 120.8 | 118.3 |
| | Median | 120.0 | 122.0 | 120.0 |
| | SD | 8.09 | 10.50 | 8.20 |
| | Min, Max | 100, 132 | 100, 144 | 102, 135 |
| Change from Baseline | n | 33 | 35 | 33 |
| to Day 2 - Discharge | Mean | −0.1 | 2.7 | −0.3 |
| | Median | −1.0 | 1.0 | −1.0 |
| | SD | 6.90 | 9.54 | 8.30 |
| | Min, Max | −12, 17 | −13, 34 | −17, 20 |
| Day 8 | n | 29 | 35 | 31 |
| | Mean | 119.2 | 123.2 | 118.7 |
| | Median | 121.0 | 122.0 | 120.0 |
| | SD | 11.19 | 12.30 | 6.93 |
| | Min, Max | 90, 141 | 104, 170 | 101, 129 |
| Change from Baseline | n | 29 | 35 | 31 |
| to Day 8 | Mean | 1.2 | 5.0 | −0.4 |
| | Median | 1.0 | 2.0 | −1.0 |
| | SD | 7.57 | 12.21 | 6.93 |
| | Min, Max | −12, 20 | −15, 46 | −17, 16 |
| Day 15 | n | 31 | 35 | 31 |
| | Mean | 123.4 | 120.3 | 119.5 |
| | Median | 122.0 | 121.0 | 121.0 |
| | SD | 11.08 | 11.20 | 8.11 |
| | Min, Max | 98, 147 | 99, 141 | 98, 134 |
| Change from Baseline | n | 31 | 35 | 31 |
| to Day 15 | Mean | 5.2 | 2.1 | 0.4 |
| | Median | 3.0 | 0.0 | −2.0 |
| | SD | 10.29 | 10.09 | 8.56 |
| | Min, Max | −14, 26 | −16, 29 | −23, 16 |

TABLE 14
Diastolic Blood Pressure (mmHg).

| | | 30 mg | 60 mg | Placebo |
|---|---|---|---|---|
| Time point | Statistic | (n = 33) | (n = 35) | (n = 34) |
| Baseline | n | 33 | 35 | 34 |
| | Mean | 76.7 | 75.7 | 75.8 |
| | Median | 77.0 | 78.0 | 75.0 |
| | SD | 7.71 | 7.35 | 6.80 |
| | Min, Max | 56, 92 | 65, 87 | 60, 88 |
| Day 1, 15 minutes | n | 13 | 12 | 16 |
| | Mean | 77.4 | 78.8 | 77.8 |
| | Median | 79.0 | 79.5 | 80.0 |
| | SD | 7.05 | 5.17 | 4.58 |
| | Min, Max | 62, 88 | 70, 85 | 70, 83 |
| Change from Baseline | n | 13 | 12 | 16 |
| to Day 1, 15 minutes | Mean | 0.5 | 2.2 | −0.8 |
| | Median | 2.0 | 1.5 | −1.0 |
| | SD | 3.55 | 3.90 | 2.17 |
| | Min, Max | −5, 8 | −2, 10 | −4, 3 |
| Day 1, 40 minutes | n | 33 | 35 | 33 |
| | Mean | 77.7 | 78.1 | 75.6 |
| | Median | 81.0 | 78.0 | 77.0 |
| | SD | 9.33 | 8.17 | 7.68 |
| | Min, Max | 52, 93 | 65, 98 | 57, 96 |
| Change from Baseline | n | 33 | 35 | 33 |
| to Day 1, 40 minutes | Mean | 1.0 | 2.4 | 0.0 |
| | Median | 1.0 | 2.0 | 0.0 |
| | SD | 6.84 | 5.61 | 3.89 |
| | Min, Max | −15, 17 | −6, 17 | −8, 8 |
| Day 1, 60 minutes | n | 14 | 13 | 17 |
| | Mean | 75.4 | 79.5 | 78.2 |
| | Median | 75.0 | 77.0 | 80.0 |
| | SD | 6.70 | 5.58 | 5.64 |
| | Min, Max | 63, 85 | 71, 88 | 68, 87 |
| Change from Baseline | n | 14 | 13 | 17 |
| to Day 1, 60 minutes | Mean | −1.2 | 3.2 | −0.1 |
| | Median | −1.5 | 4.0 | −1.0 |
| | SD | 4.23 | 4.23 | 3.89 |
| | Min, Max | −7, 7 | −4, 11 | −5, 9 |
| Day 1, 2 hours | n | 33 | 35 | 33 |
| | Mean | 77.3 | 75.7 | 75.1 |
| | Median | 78.0 | 75.0 | 75.0 |
| | SD | 7.42 | 6.17 | 6.64 |
| | Min, Max | 66, 96 | 61, 87 | 57, 94 |
| Change from Baseline | n | 33 | 35 | 33 |
| to Day 1, 2 hours | Mean | 0.5 | −0.1 | −0.5 |
| | Median | −1.0 | 0.0 | −1.0 |
| | SD | 5.60 | 4.54 | 5.66 |
| | Min, Max | −9, 13 | −7, 11 | −14, 14 |
| Day 1, 4 hours | n | 33 | 35 | 33 |
| | Mean | 76.1 | 75.1 | 74.5 |
| | Median | 76.0 | 74.0 | 75.0 |
| | SD | 7.74 | 6.43 | 8.58 |
| | Min, Max | 62, 92 | 65, 89 | 55, 103 |
| Change from Baseline | n | 33 | 35 | 33 |
| to Day 1, 4 hours | Mean | −0.6 | −0.6 | −1.1 |
| | Median | −1.0 | 0.0 | −1.0 |
| | SD | 5.39 | 6.63 | 6.43 |
| | Min, Max | −12, 14 | −15, 13 | −16, 15 |
| Day 1, 6 hours | n | 33 | 34 | 31 |
| | Mean | 75.2 | 75.8 | 74.6 |
| | Median | 75.0 | 76.5 | 76.0 |
| | SD | 7.34 | 5.92 | 6.44 |
| | Min, Max | 56, 88 | 63, 85 | 62, 86 |
| Change from Baseline | n | 33 | 34 | 31 |
| to Day 1, 6 hours | Mean | −1.5 | −0.0 | −1.2 |
| | Median | −3.0 | −1.0 | −2.0 |
| | SD | 4.87 | 4.99 | 4.85 |
| | Min, Max | −10, 11 | −10, 10 | −8, 12 |
| Day 2 - Discharge | n | 33 | 35 | 33 |
| | Mean | 74.7 | 77.2 | 76.6 |
| | Median | 75.0 | 77.0 | 76.0 |
| | SD | 7.09 | 7.42 | 6.89 |
| | Min, Max | 56, 87 | 65, 92 | 55, 93 |
| Change from Baseline | n | 33 | 35 | 33 |
| to Day 2 - Discharge | Mean | −2.0 | 1.5 | 1.0 |
| | Median | −2.0 | 1.0 | 0.0 |
| | SD | 6.68 | 8.52 | 5.98 |
| | Min, Max | −21, 16 | −19, 27 | −17, 15 |
| Day 8 | n | 29 | 35 | 31 |
| | Mean | 75.8 | 78.7 | 75.6 |
| | Median | 78.0 | 78.0 | 76.0 |
| | SD | 7.78 | 7.63 | 5.71 |
| | Min, Max | 60, 92 | 62, 102 | 63, 89 |
| Change from Baseline | n | 29 | 35 | 31 |
| to Day 8 | Mean | −1.0 | 3.0 | −0.0 |
| | Median | −1.0 | 3.0 | −1.0 |
| | SD | 4.70 | 7.89 | 5.36 |
| | Min, Max | −7, 7 | −12, 26 | −8, 13 |
| Day 15 | n | 31 | 35 | 31 |
| | Mean | 78.9 | 77.5 | 76.6 |
| | Median | 79.0 | 78.0 | 76.0 |
| | SD | 9.09 | 5.77 | 6.88 |
| | Min, Max | 62, 106 | 67, 88 | 58, 98 |
| Change from Baseline | n | 31 | 35 | 31 |
| to Day 15 | Mean | 2.1 | 1.7 | 1.0 |
| | Median | 1.0 | 1.0 | 0.0 |
| | SD | 9.21 | 4.88 | 6.01 |
| | Min, Max | −22, 30 | −9, 14 | −10, 15 |

TABLE 15
Pulse (beats/minute).

| | | 30 mg | 60 mg | Placebo |
|---|---|---|---|---|
| Time point | Statistic | (n = 33) | (n = 35) | (n = 34) |
| Baseline | n | 33 | 35 | 34 |
| | Mean | 70.5 | 72.7 | 71.6 |
| | Median | 70.0 | 71.0 | 70.0 |
| | SD | 10.12 | 11.81 | 9.47 |
| | Min, Max | 50, 88 | 49, 100 | 50, 93 |
| Day 1, 15 minutes | n | 13 | 12 | 16 |
| | Mean | 69.4 | 67.3 | 72.6 |
| | Median | 70.0 | 66.5 | 72.0 |
| | SD | 7.51 | 8.54 | 5.25 |
| | Min, Max | 55, 82 | 52, 83 | 65, 84 |
| Change from Baseline | n | 13 | 12 | 16 |
| to Day 1, 15 minutes | Mean | −0.4 | 0.0 | −0.5 |
| | Median | 0.0 | 0.5 | 0.5 |
| | SD | 6.19 | 5.70 | 5.56 |
| | Min, Max | −12, 10 | −11, 11 | −14, 6 |
| Day 1, 40 minutes | n | 33 | 35 | 33 |
| | Mean | 69.9 | 68.0 | 70.2 |
| | Median | 70.0 | 68.0 | 70.0 |
| | SD | 10.19 | 11.37 | 8.37 |
| | Min, Max | 52, 86 | 49, 102 | 46, 83 |
| Change from Baseline | n | 33 | 35 | 33 |
| to Day 1, 40 minutes | Mean | −0.6 | −4.7 | −1.9 |
| | Median | −1.0 | −5.0 | −2.0 |
| | SD | 7.13 | 6.29 | 6.01 |
| | Min, Max | −15, 17 | −19, 5 | −17, 7 |
| Day 1, 60 minutes | n | 14 | 13 | 17 |
| | Mean | 67.2 | 64.9 | 72.1 |
| | Median | 68.5 | 65.0 | 72.0 |
| | SD | 8.67 | 7.53 | 4.32 |
| | Min, Max | 53, 82 | 53, 76 | 64, 80 |
| Change from Baseline | n | 14 | 13 | 17 |
| to Day 1, 60 minutes | Mean | −2.5 | −2.3 | −0.7 |
| | Median | −3.5 | −1.0 | 0.0 |
| | SD | 6.27 | 4.89 | 7.45 |
| | Min, Max | −13, 8 | −12, 4 | −19, 9 |
| Day 1, 2 hours | n | 33 | 35 | 33 |
| | Mean | 70.7 | 68.2 | 70.8 |
| | Median | 70.0 | 70.0 | 69.0 |
| | SD | 11.87 | 10.64 | 8.88 |
| | Min, Max | 53, 100 | 44, 100 | 50, 96 |
| Change from Baseline | n | 33 | 35 | 33 |
| to Day 1, 2 hours | Mean | 0.2 | −4.5 | −1.3 |
| | Median | −2.0 | −5.0 | 0.0 |
| | SD | 8.59 | 6.58 | 7.44 |
| | Min, Max | −16, 18 | −21, 13 | −22, 13 |
| Day 1, 4 hours | n | 33 | 35 | 33 |
| | Mean | 72.1 | 70.7 | 71.8 |
| | Median | 72.0 | 72.0 | 71.0 |
| | SD | 10.85 | 11.08 | 8.74 |
| | Min, Max | 52, 95 | 48, 108 | 55, 93 |
| Change from Baseline | n | 33 | 35 | 33 |
| to Day 1, 4 hours | Mean | 1.6 | −2.0 | −0.3 |
| | Median | 2.0 | −1.0 | 0.0 |
| | SD | 6.41 | 6.82 | 6.31 |
| | Min, Max | −13, 14 | −20, 15 | −22, 10 |
| Day 1, 6 hours | n | 33 | 34 | 31 |
| | Mean | 70.7 | 72.6 | 72.8 |
| | Median | 72.0 | 71.0 | 73.0 |
| | SD | 9.41 | 12.09 | 8.90 |
| | Min, Max | 53, 86 | 52, 119 | 50, 92 |
| Change from Baseline | n | 33 | 34 | 31 |
| to Day 1, 6 hours | Mean | 0.2 | −0.3 | 0.5 |
| | Median | 1.0 | 0.5 | 1.0 |
| | SD | 7.77 | 7.27 | 5.78 |
| | Min, Max | −20, 16 | −18, 19 | −15, 11 |
| Day 2 - Discharge | n | 33 | 35 | 33 |
| | Mean | 73.5 | 72.2 | 74.3 |
| | Median | 75.0 | 70.0 | 72.0 |
| | SD | 10.87 | 12.61 | 10.35 |
| | Min, Max | 52, 109 | 47, 121 | 45, 98 |
| Change from Baseline | n | 33 | 35 | 33 |
| to Day 2 - Discharge | Mean | 3.1 | −0.6 | 2.2 |
| | Median | 2.0 | −1.0 | 2.0 |
| | SD | 7.20 | 8.47 | 7.91 |
| | Min, Max | −11, 24 | −22, 21 | −14, 21 |
| Day 8 | n | 29 | 35 | 31 |
| | Mean | 74.1 | 73.3 | 72.5 |
| | Median | 74.0 | 70.0 | 72.0 |
| | SD | 10.48 | 12.17 | 10.97 |
| | Min, Max | 54, 93 | 56, 118 | 47, 99 |
| Change from Baseline | n | 29 | 35 | 31 |
| to Day 8 | Mean | 2.1 | 0.6 | 0.0 |
| | Median | 1.0 | 0.0 | −1.0 |
| | SD | 10.45 | 11.33 | 9.65 |
| | Min, Max | −16, 29 | −23, 21 | −17, 22 |
| Day 15 | n | 31 | 35 | 31 |
| | Mean | 74.4 | 71.0 | 71.1 |
| | Median | 75.0 | 69.0 | 70.0 |
| | SD | 11.01 | 9.38 | 9.70 |
| | Min, Max | 52, 94 | 56, 97 | 53, 92 |
| Change from Baseline | n | 31 | 35 | 31 |
| to Day 15 | Mean | 3.3 | −1.7 | −1.4 |
| | Median | 2.0 | 0.0 | −2.0 |
| | SD | 10.26 | 8.75 | 8.75 |
| | Min, Max | −16, 26 | −21, 15 | −16, 19 |

TABLE 16
Temperature (° C.).

| | | 30 mg | 60 mg | Placebo |
|---|---|---|---|---|
| Time point | Statistic | (n = 33) | (n = 35) | (n = 34) |
| Baseline | n | 33 | 35 | 34 |
| | Mean | 36.54 | 36.53 | 36.56 |
| | Median | 36.50 | 36.50 | 36.50 |
| | SD | 0.212 | 0.217 | 0.222 |
| | Min, Max | 36.0, 37.2 | 35.8, 37.0 | 35.8, 37.0 |
| Day 1, 15 minutes | n | 13 | 12 | 16 |
| | Mean | 36.57 | 36.58 | 36.48 |
| | Median | 36.50 | 36.50 | 36.50 |
| | SD | 0.210 | 0.147 | 0.083 |
| | Min, Max | 36.4, 37.2 | 36.4, 36.9 | 36.4, 36.7 |
| Change from Baseline | n | 13 | 12 | 16 |
| to Day 1, 15 minutes | Mean | 0.05 | 0.04 | −0.02 |
| | Median | 0.10 | 0.00 | 0.00 |
| | SD | 0.151 | 0.131 | 0.058 |
| | Min, Max | −0.3, 0.4 | −0.2, 0.3 | −0.1, 0.1 |
| Day 1, 40 minutes | n | 33 | 35 | 33 |
| | Mean | 36.58 | 36.58 | 36.60 |
| | Median | 36.60 | 36.60 | 36.50 |
| | SD | 0.205 | 0.134 | 0.180 |
| | Min, Max | 36.2, 37.2 | 36.3, 36.8 | 36.3, 37.1 |
| Change from Baseline | n | 33 | 35 | 33 |
| to Day 1, 40 minutes | Mean | 0.05 | 0.05 | 0.06 |
| | Median | 0.00 | 0.00 | 0.00 |
| | SD | 0.139 | 0.211 | 0.255 |
| | Min, Max | −0.2, 0.5 | −0.4, 0.7 | −0.3, 1.0 |
| Day 1, 60 minutes | n | 14 | 13 | 17 |
| | Mean | 36.57 | 36.55 | 36.51 |
| | Median | 36.50 | 36.50 | 36.50 |
| | SD | 0.213 | 0.133 | 0.090 |
| | Min, Max | 36.3, 37.2 | 36.4, 36.8 | 36.3, 36.6 |
| Change from Baseline | n | 14 | 13 | 17 |
| to Day 1, 60 minutes | Mean | 0.05 | 0.01 | 0.00 |
| | Median | 0.00 | 0.00 | 0.00 |
| | SD | 0.183 | 0.155 | 0.106 |
| | Min, Max | −0.3, 0.5 | −0.3, 0.3 | −0.2, 0.2 |
| Day 1, 2 hours | n | 33 | 35 | 33 |
| | Mean | 36.62 | 36.60 | 36.61 |
| | Median | 36.60 | 36.60 | 36.60 |
| | SD | 0.153 | 0.150 | 0.204 |
| | Min, Max | 36.4, 37.2 | 36.2, 37.0 | 36.3, 37.3 |
| Change from Baseline | n | 33 | 35 | 33 |
| to Day 1, 2 hours | Mean | 0.08 | 0.07 | 0.07 |
| | Median | 0.10 | 0.00 | 0.00 |
| | SD | 0.182 | 0.248 | 0.243 |
| | Min, Max | −0.2, 0.7 | −0.5, 0.8 | −0.2, 0.8 |
| Day 1, 4 hours | n | 33 | 35 | 33 |
| | Mean | 36.57 | 36.60 | 36.60 |
| | Median | 36.60 | 36.60 | 36.60 |
| | SD | 0.173 | 0.153 | 0.203 |
| | Min, Max | 36.3, 37.1 | 36.3, 37.0 | 36.3, 37.3 |
| Change from Baseline | n | 33 | 35 | 33 |
| to Day 1, 4 hours | Mean | 0.03 | 0.07 | 0.06 |
| | Median | 0.00 | 0.00 | 0.00 |
| | SD | 0.166 | 0.245 | 0.263 |
| | Min, Max | −0.4, 0.5 | −0.2, 0.9 | −0.4, 1.1 |
| Day 1, 6 hours | n | 33 | 34 | 31 |
| | Mean | 36.63 | 36.61 | 36.68 |
| | Median | 36.60 | 36.60 | 36.60 |
| | SD | 0.208 | 0.200 | 0.272 |
| | Min, Max | 36.3, 37.2 | 36.1, 37.1 | 36.2, 37.5 |
| Change from Baseline | n | 33 | 34 | 31 |
| to Day 1, 6 hours | Mean | 0.09 | 0.07 | 0.13 |
| | Median | 0.10 | 0.00 | 0.10 |
| | SD | 0.260 | 0.234 | 0.364 |
| | Min, Max | −0.4, 0.9 | −0.4, 1.0 | −0.6, 1.2 |
| Day 2 - Discharge | n | 33 | 35 | 33 |
| | Mean | 36.59 | 36.60 | 36.60 |
| | Median | 36.60 | 36.60 | 36.60 |
| | SD | 0.240 | 0.194 | 0.207 |
| | Min, Max | 35.9, 37.1 | 36.1, 36.9 | 36.1, 37.3 |
| Change from Baseline | n | 33 | 35 | 33 |
| to Day 2 - Discharge | Mean | 0.05 | 0.07 | 0.06 |
| | Median | 0.00 | 0.10 | 0.00 |
| | SD | 0.189 | 0.224 | 0.329 |
| | Min, Max | −0.3, 0.5 | −0.7, 0.6 | −0.7, 1.0 |
| Day 8 | n | 29 | 35 | 31 |
| | Mean | 36.50 | 36.55 | 36.59 |
| | Median | 36.50 | 36.60 | 36.60 |
| | SD | 0.148 | 0.274 | 0.175 |
| | Min, Max | 36.1, 36.8 | 35.8, 37.2 | 36.3, 37.1 |
| Change from Baseline | n | 29 | 35 | 31 |
| to Day 8 | Mean | −0.03 | 0.01 | 0.05 |
| | Median | 0.00 | 0.00 | 0.00 |
| | SD | 0.233 | 0.364 | 0.295 |
| | Min, Max | −0.7, 0.6 | −0.9, 1.2 | −0.5, 1.0 |
| Day 15 | n | 31 | 35 | 31 |
| | Mean | 36.60 | 36.62 | 36.58 |
| | Median | 36.60 | 36.60 | 36.60 |
| | SD | 0.211 | 0.181 | 0.171 |
| | Min, Max | 36.2, 37.1 | 36.3, 37.0 | 36.4, 37.0 |
| Change from Baseline | n | 31 | 35 | 31 |
| to Day 15 | Mean | 0.05 | 0.08 | 0.04 |
| | Median | 0.00 | 0.00 | 0.00 |
| | SD | 0.241 | 0.313 | 0.263 |
| | Min, Max | −0.4, 0.9 | −0.5, 1.2 | −0.4, 1.2 |

TABLE 17
Respiratory Rate (breaths/minute).

| | | 30 mg | 60 mg | Placebo |
|---|---|---|---|---|
| Time point | Statistic | (n = 33) | (n = 35) | (n = 34) |
| Baseline | n | 33 | 35 | 34 |
| | Mean | 15.9 | 15.9 | 16.0 |
| | Median | 16.0 | 16.0 | 16.0 |
| | SD | 1.41 | 1.94 | 1.18 |
| | Min, Max | 14, 20 | 12, 20 | 14, 19 |
| Day 1, 15 minutes | n | 13 | 12 | 16 |
| | Mean | 16.0 | 15.5 | 16.2 |
| | Median | 16.0 | 15.0 | 16.0 |
| | SD | 2.04 | 1.31 | 1.05 |
| | Min, Max | 14, 22 | 14, 18 | 14, 18 |
| Change from Baseline | n | 13 | 12 | 16 |
| to Day 1, 15 minutes | Mean | 0.1 | −0.6 | 0.1 |
| | Median | 0.0 | −1.0 | 0.0 |
| | SD | 1.38 | 1.56 | 0.96 |
| | Min, Max | −3, 3 | −4, 2 | −1, 2 |
| Day 1, 40 minutes | n | 33 | 35 | 33 |
| | Mean | 15.7 | 15.9 | 15.7 |
| | Median | 16.0 | 16.0 | 16.0 |
| | SD | 1.07 | 1.90 | 0.99 |
| | Min, Max | 13, 18 | 12, 23 | 14, 18 |
| Change from Baseline | n | 33 | 35 | 33 |
| to Day 1, 40 minutes | Mean | −0.2 | −0.0 | −0.3 |
| | Median | 0.0 | 0.0 | 0.0 |
| | SD | 1.54 | 1.58 | 1.16 |
| | Min, Max | −5, 2 | −4, 4 | −4, 1 |
| Day 1, 60 minutes | n | 14 | 13 | 17 |
| | Mean | 15.4 | 15.2 | 15.5 |
| | Median | 15.5 | 16.0 | 16.0 |
| | SD | 1.02 | 1.21 | 1.37 |
| | Min, Max | 14, 17 | 12, 16 | 14, 18 |
| Change from Baseline | n | 14 | 13 | 17 |
| to Day 1, 60 minutes | Mean | −0.4 | −0.8 | −0.5 |
| | Median | 0.0 | −1.0 | 0.0 |
| | SD | 1.83 | 1.28 | 1.55 |
| | Min, Max | −5, 3 | −4, 1 | −3, 1 |
| Day 1, 2 hours | n | 33 | 35 | 33 |
| | Mean | 15.9 | 15.5 | 15.8 |
| | Median | 16.0 | 16.0 | 16.0 |
| | SD | 1.24 | 1.48 | 1.23 |
| | Min, Max | 13, 18 | 11, 19 | 14, 18 |
| Change from Baseline | n | 33 | 35 | 33 |
| to Day 1, 2 hours | Mean | −0.1 | −0.5 | −0.1 |
| | Median | 0.0 | 0.0 | 0.0 |
| | SD | 1.25 | 1.44 | 1.24 |
| | Min, Max | −2, 3 | −4, 2 | −4, 2 |
| Day 1, 4 hours | n | 33 | 35 | 33 |
| | Mean | 16.1 | 15.9 | 16.1 |
| | Median | 16.0 | 16.0 | 16.0 |
| | SD | 1.49 | 1.33 | 1.22 |
| | Min, Max | 14, 21 | 12, 20 | 14, 20 |
| Change from Baseline | n | 33 | 35 | 33 |
| to Day 1, 4 hours | Mean | 0.2 | −0.1 | 0.2 |
| | Median | 0.0 | 0.0 | 0.0 |
| | SD | 1.13 | 1.42 | 1.18 |
| | Min, Max | −2, 2 | −4, 3 | −3, 4 |
| Day 1, 6 hours | n | 33 | 34 | 31 |
| | Mean | 15.9 | 15.7 | 16.0 |
| | Median | 16.0 | 16.0 | 16.0 |
| | SD | 1.23 | 1.85 | 1.35 |
| | Min, Max | 14, 18 | 12, 20 | 14, 20 |
| Change from Baseline | n | 33 | 34 | 31 |
| to Day 1, 6 hours | Mean | −0.0 | −0.2 | 0.0 |
| | Median | 0.0 | 0.0 | 0.0 |
| | SD | 1.24 | 2.05 | 1.39 |
| | Min, Max | −3, 2 | −8, 6 | −2, 4 |
| Day 2 - Discharge | n | 33 | 35 | 33 |
| | Mean | 15.9 | 15.9 | 16.0 |
| | Median | 16.0 | 16.0 | 16.0 |
| | SD | 1.36 | 1.36 | 0.94 |
| | Min, Max | 14, 21 | 12, 20 | 15, 18 |
| Change from Baseline | n | 33 | 35 | 33 |
| to Day 2 - Discharge | Mean | −0.1 | −0.0 | 0.0 |
| | Median | 0.0 | 0.0 | 0.0 |
| | SD | 1.09 | 1.34 | 1.02 |
| | Min, Max | −4, 1 | −4, 4 | −2, 2 |
| Day 8 | n | 29 | 35 | 31 |
| | Mean | 15.9 | 15.7 | 15.8 |
| | Median | 16.0 | 16.0 | 16.0 |
| | SD | 1.39 | 1.58 | 1.36 |
| | Min, Max | 14, 18 | 12, 18 | 12, 18 |
| Change from Baseline | n | 29 | 35 | 31 |
| to Day 8 | Mean | −0.1 | −0.2 | −0.3 |
| | Median | 0.0 | 0.0 | 0.0 |
| | SD | 1.60 | 1.93 | 1.24 |
| | Min, Max | −5, 3 | −6, 4 | −4, 2 |
| Day 15 | n | 31 | 35 | 31 |
| | Mean | 15.7 | 15.8 | 15.6 |
| | Median | 16.0 | 16.0 | 15.0 |
| | SD | 1.10 | 1.60 | 1.17 |
| | Min, Max | 14, 18 | 12, 20 | 13, 18 |
| Change from Baseline | n | 31 | 35 | 31 |
| to Day 15 | Mean | −0.3 | −0.1 | −0.4 |
| | Median | 0.0 | 0.0 | 0.0 |
| | SD | 1.75 | 1.76 | 1.09 |
| | Min, Max | −6, 1 | −3, 6 | −2, 2 |

TABLE 18
Oxygen Saturation (%).

| | | 30 mg | 60 mg | Placebo |
|---|---|---|---|---|
| Time point | Statistic | (n = 33) | (n = 35) | (n = 34) |
| Baseline | n | 33 | 35 | 33 |
| | Mean | 98.6 | 98.2 | 98.3 |
| | Median | 99.0 | 98.0 | 99.0 |
| | SD | 0.82 | 1.10 | 1.02 |
| | Min, Max | 96, 100 | 95, 100 | 95, 99 |
| Day 1, 15 minutes | n | 27 | 23 | 26 |
| | Mean | 98.3 | 98.2 | 98.2 |
| | Median | 98.0 | 98.0 | 98.5 |
| | SD | 0.91 | 1.07 | 1.03 |
| | Min, Max | 96, 100 | 96, 100 | 96, 100 |
| Change from Baseline | n | 27 | 23 | 25 |
| to Day 1, 15 minutes | Mean | −0.3 | 0.0 | −0.1 |
| | Median | 0.0 | 0.0 | 0.0 |
| | SD | 0.96 | 1.17 | 0.91 |
| | Min, Max | −3, 2 | −2, 4 | −2, 2 |
| Day 1, 40 minutes | n | 33 | 35 | 33 |
| | Mean | 98.3 | 98.1 | 97.8 |
| | Median | 98.0 | 98.0 | 98.0 |
| | SD | 0.76 | 0.97 | 1.18 |
| | Min, Max | 96, 99 | 95, 100 | 95, 99 |
| Change from Baseline | n | 33 | 35 | 32 |
| to Day 1, 40 minutes | Mean | −0.4 | −0.1 | −0.4 |
| | Median | 0.0 | 0.0 | 0.0 |
| | SD | 0.90 | 1.08 | 1.27 |
| | Min, Max | −2, 2 | −2, 2 | −3, 3 |
| Day 1, 60 minutes | n | 28 | 26 | 27 |
| | Mean | 98.2 | 98.1 | 97.6 |
| | Median | 98.0 | 98.0 | 98.0 |
| | SD | 0.98 | 1.06 | 1.21 |
| | Min, Max | 96, 100 | 95, 100 | 95, 99 |
| Change from Baseline | n | 28 | 26 | 26 |
| to Day 1, 60 minutes | Mean | −0.5 | −0.1 | −0.7 |
| | Median | 0.0 | 0.0 | −1.0 |
| | SD | 1.04 | 1.40 | 1.02 |
| | Min, Max | −3, 1 | −4, 3 | −3, 1 |
| Day 1, 2 hours | n | 33 | 35 | 33 |
| | Mean | 98.4 | 98.2 | 98.1 |
| | Median | 99.0 | 98.0 | 98.0 |
| | SD | 0.83 | 0.71 | 1.17 |
| | Min, Max | 97, 100 | 97, 100 | 95, 100 |
| Change from Baseline | n | 33 | 35 | 32 |
| to Day 1, 2 hours | Mean | −0.2 | 0.0 | −0.2 |
| | Median | 0.0 | 0.0 | 0.0 |
| | SD | 1.02 | 1.06 | 0.91 |
| | Min, Max | −2, 3 | −2, 3 | −2, 3 |
| Day 1, 4 hours | n | 33 | 32 | 30 |
| | Mean | 98.3 | 98.3 | 98.0 |
| | Median | 98.0 | 99.0 | 99.0 |
| | SD | 0.91 | 1.09 | 1.45 |
| | Min, Max | 96, 100 | 96, 100 | 94, 100 |
| Change from Baseline | n | 33 | 32 | 29 |
| to Day 1, 4 hours | Mean | −0.4 | 0.2 | −0.4 |
| | Median | 0.0 | 0.0 | 0.0 |
| | SD | 1.06 | 0.88 | 1.21 |
| | Min, Max | −3, 3 | −2, 1 | −3, 2 |

Tables 19-24 summarize adverse events that occurred during the study. There were no serious adverse events (SAEs).

TABLE 19
Overall Summary of Adverse Events.

| | 30 mg | | 60 mg | | Placebo | | Total | |
|---|---|---|---|---|---|---|---|---|
| Number of subjects with | (n = 33) | | (n = 35) | | (n = 34) | | (n = 102) | |
| Any adverse events (AE) | 11 | (33) | 19 | (54) | 18 | (53) | 48 | (47) |
| Treatment-emergent adverse | 11 | (33) | 18 | (51) | 18 | (53) | 47 | (46) |
| events (TEAE) | | | | | | | | |
| Mild | 7 | (21) | 9 | (26) | 14 | (41) | 30 | (29) |
| Moderate | 4 | (12) | 7 | (20) | 4 | (12) | 15 | (15) |
| Severe | 0 | (0) | 2 | (6) | 0 | (0) | 2 | (2) |
| Treatment-emergent serious | 0 | (0) | 0 | (0) | 0 | (0) | 0 | (0) |
| adverse events (SAE) | | | | | | | | |
| Mild | 0 | (0) | 0 | (0) | 0 | (0) | 0 | (0) |
| Moderate | 0 | (0) | 0 | (0) | 0 | (0) | 0 | (0) |
| Severe | 0 | (0) | 0 | (0) | 0 | (0) | 0 | (0) |
| Treatment-related TEAEs | 10 | (30) | 16 | (46) | 16 | (47) | 42 | (41) |
| Treatment-related treatment- | 0 | (0) | 0 | (0) | 0 | (0) | 0 | (0) |
| emergent SAEs | | | | | | | | |
| TEAE with outcome of death | 0 | (0) | 0 | (0) | 0 | (0) | 0 | (0) |
| Treatment-related and severe | 0 | (0) | 2 | (6) | 0 | (0) | 2 | (2) |
| TEAEs | | | | | | | | |
| TEAEs resulting in treatment | 0 | (0) | 0 | (0) | 1 | (3) | 1 | (1) |
| discontinuation [n(%)] | | | | | | | | |
| Serious TEAEs resulting in | 0 | (0) | 0 | (0) | 0 | (0) | 0 | (0) |
| treatment discontinuation [n(%)] | | | | | | | | |
| TEAEs resulting in discontinuation | 0 | (0) | 0 | (0) | 1 | (3) | 1 | (1) |
| from study [n(%)] | | | | | | | | |
| TEAEs of COVID-19 [n(%)] | 0 | (0) | 0 | (0) | 0 | (0) | 0 | (0) |

In Table 19, frequencies are numbers of subjects experiencing at least one adverse event in that category. Subjects experiencing more than one adverse event in each category are counted only once for that category. Adverse events which are possibly related, probably related, definitely related or with unknown relationship with study drug are considered ‘Related’ for analysis. Any treatment-emergent adverse events which were not graded are assumed to be ‘Severe’. Subjects experiencing a treatment-emergent adverse event at multiple times will be counted only once at the maximum severity.

TABLE 20
Incidence of MOASS and CADSS Categories.

| | 30 mg | | 60 mg | | Placebo | | Total |
|---|---|---|---|---|---|---|---|
| | (n = 33) | | (n = 35) | | (n = 33) | | (n = 101) |
| Number of subjects with at | 33 | | 35 | | 33 | | 101 |
| least one post-baseline | | | | | | | |
| MOASS or CADSS assessment | | | | | | | |
| Subjects with post-baseline | 9 | (27.3) | 12 | (34.3) | 11 | (33.3) | 32 (31.7) |
| MOASS <5 [n(%)] | | | | | | | |
| Subjects with post-baseline | 12 | (36.4) | 8 | (22.9) | 9 | (27.3) | 29 (28.7) |
| CADSS >4 and change from | | | | | | | |
| baseline >0 [n(%)] | | | | | | | |

TABLE 21
Incidence of Clinically Important Blood Pressure Categories.

| | 30 mg | 60 mg | Placebo | Total |
|---|---|---|---|---|
| | (n = 33) | (n = 35) | (n = 34) | (n = 102) |
| Number of subjects with at least | 33 | 35 | 33 | 101 |
| one post-baseline systolic or | | | | |
| diastolic blood pressure assessment | | | | |

Subjects with clinically important blood pressure categories: [n(%)]

| | | | | |
|---|---|---|---|---|
| Post-baseline systolic blood | 0 (0.0) | 0 (0.0) | 0 (0.0) | 0 (0.0) |
| pressure >180 mmHg | | | | |
| Change from baseline systolic | 0 (0.0) | 1 (2.9) | 0 (0.0) | 1 (1.0) |
| blood pressure >40 mmHg | | | | |
| Post-baseline diastolic blood | 0 (0.0) | 0 (0.0) | 0 (0.0) | 0 (0.0) |
| pressure >110 mmHg | | | | |
| Change from baseline diastolic | 1 (3.0) | 1 (2.9) | 0 (0.0) | 2 (2.0) |
| blood pressure >25 mmHg | | | | |

TABLE 22
Summary of Treatment-Emergent Adverse Events
by System Organ Class and Preferred Term.

| | 30 mg | | 60 mg | | Placebo | | Total | |
|---|---|---|---|---|---|---|---|---|
| | (n = 33) | | (n = 35) | | (n = 34) | | (n = 102) | |
| Number of treatment-emergent | 22 | | 53 | | 44 | | 119 | |
| adverse events | | | | | | | | |
| Number of subjects with any | 11 | (33) | 18 | (51) | 18 | (53) | 47 | (46) |
| treatment-emergent adverse | | | | | | | | |
| events | | | | | | | | |
| Nervous system disorders | 9 | (27) | 14 | (40) | 15 | (44) | 38 | (37) |
| Somnolence | 4 | (12) | 4 | (11) | 9 | (26) | 17 | (17) |
| Dizziness | 2 | (6) | 7 | (20) | 5 | (15) | 14 | (14) |
| Headache | 3 | (9) | 2 | (6) | 3 | (9) | 8 | (8) |
| Dysarthria | 0 | (0) | 2 | (6) | 0 | (0) | 2 | (2) |
| Paraesthesia | 0 | (0) | 2 | (6) | 0 | (0) | 2 | (2) |
| Balance disorder | 0 | (0) | 0 | (0) | 1 | (3) | 1 | (1) |
| Hypoaesthesia | 0 | (0) | 0 | (0) | 1 | (3) | 1 | (1) |
| Lethargy | 0 | (0) | 1 | (3) | 0 | (0) | 1 | (1) |
| Memory impairment | 0 | (0) | 0 | (0) | 1 | (3) | 1 | (1) |
| Sedation | 0 | (0) | 1 | (3) | 0 | (0) | 1 | (1) |
| Sensory disturbance | 1 | (3) | 0 | (0) | 0 | (0) | 1 | (1) |
| Slow speech | 0 | (0) | 0 | 0) | 1 | (3) | 1 | (1) |
| Psychiatric disorders | 4 | (12) | 5 | (14) | 5 | (15) | 14 | (14) |
| Derealisation | 4 | (12) | 3 | (9) | 4 | (12) | 11 | (11) |
| Confusional state | 0 | (0) | 1 | (3) | 1 | (3) | 2 | (2) |
| Aversion | 0 | (0) | 0 | (0) | 1 | (3) | 1 | (1) |
| Bruxism | 0 | (0) | 0 | (0) | 1 | (3) | 1 | (1) |
| Dissociation | 0 | (0) | 0 | (0) | 1 | (3) | 1 | (1) |
| Euphoric mood | 0 | (0) | 1 | (3) | 0 | (0) | 1 | (1) |
| Logorrhoea | 0 | (0) | 0 | (0) | 1 | (3) | 1 | (1) |
| Time perception altered | 0 | (0) | 1 | (3) | 0 | (0) | 1 | (1) |
| Gastrointestinal disorders | 0 | (0) | 5 | (14) | 2 | (6) | 7 | (7) |
| Nausea | 0 | (0) | 3 | (9) | 1 | (3) | 4 | (4) |
| Dry mouth | 0 | (0) | 1 | (3) | 1 | (3) | 2 | (2) |
| Diarrhoea | 0 | (0) | 0 | (0) | 1 | (3) | 1 | (1) |
| Hypoaesthesia oral | 0 | (0) | 1 | (3) | 0 | (0) | 1 | (1) |
| General disorders and | 1 | (3) | 4 | (11) | 1 | (3) | 6 | (6) |
| administration site conditions | | | | | | | | |
| Feeling drunk | 0 | (0) | 2 | (6) | 0 | (0) | 2 | (2) |
| Energy increased | 0 | (0) | 1 | (3) | 0 | (0) | 1 | (1) |
| Fatigue | 0 | (0) | 1 | (3) | 0 | (0) | 1 | (1) |
| Feeling abnormal | 0 | (0) | 1 | (3) | 0 | (0) | 1 | (1) |
| Influenza like illness | 0 | (0) | 0 | (0) | 1 | (3) | 1 | (1) |
| Infusion site hypoaesthesia | 0 | (0) | 1 | (3) | 0 | (0) | 1 | (1) |
| Vessel puncture site erythema | 1 | (3) | 0 | (0) | 0 | (0) | 1 | (1) |
| Cardiac disorders | 0 | (0) | 2 | (6) | 1 | (3) | 3 | (3) |
| Defect conduction intraventricular | 0 | (0) | 1 | (3) | 0 | (0) | 1 | (1) |
| Left ventricular hypertrophy | 0 | (0) | 1 | (3) | 0 | (0) | 1 | (1) |
| Tachycardia | 0 | (0) | 0 | (0) | 1 | (3) | 1 | (1) |
| Injury, poisoning and | 1 | (3) | 1 | (3) | 1 | (3) | 3 | (3) |
| procedural complications | | | | | | | | |
| Muscle strain | 1 | (3) | 0 | (0) | 0 | (0) | 1 | (1) |
| Skin abrasion | 0 | (0) | 1 | (3) | 0 | (0) | 1 | (1) |
| Venous injury | 0 | (0) | 0 | (0) | 1 | (3) | 1 | (1) |
| Respiratory, thoracic and | 1 | (3) | 1 | (3) | 1 | (3) | 3 | (3) |
| mediastinal disorders | | | | | | | | |
| Dyspnoea exertional | 1 | (3) | 0 | (0) | 0 | (0) | 1 | (1) |
| Rhinorrhoea | 0 | (0) | 0 | (0) | 1 | (3) | 1 | (1) |
| Throat tightness | 0 | (0) | 1 | (3) | 0 | (0) | 1 | (1) |
| Ear and labyrinth disorders | 1 | (3) | 0 | (0) | 1 | (3) | 2 | (2) |
| Hyperacusis | 0 | (0) | 0 | (0) | 1 | (3) | 1 | (1) |
| Vertigo | 1 | (3) | 0 | (0) | 0 | (0) | 1 | (1) |
| Eye disorders | 0 | (0) | 1 | (3) | 1 | (3) | 2 | (2) |
| Vision blurred | 0 | (0) | 1 | (3) | 0 | (0) | 1 | (1) |
| Visual impairment | 0 | (0) | 0 | (0) | 1 | (3) | 1 | (1) |
| Infections and infestations | 1 | (3) | 0 | (0) | 1 | (3) | 2 | (2) |
| Sinusitis | 0 | (0) | 0 | (0) | 1 | (3) | 1 | (1) |
| Urinary tract infection | 1 | (3) | 0 | (0) | 0 | (0) | 1 | (1) |
| Investigations | 0 | (0) | 1 | (3) | 1 | (3) | 2 | (2) |
| Blood pressure increased | 0 | (0) | 1 | (3) | 1 | (3) | 2 | (2) |
| Musculoskeletal and connective | 0 | (0) | 1 | (3) | 1 | (3) | 2 | (2) |
| tissue disorders | | | | | | | | |
| Limb discomfort | 0 | (0) | 0 | (0) | 1 | (3) | 1 | (1) |
| Muscular weakness | 0 | (0) | 1 | (3) | 0 | (0) | 1 | (1) |
| Skin and subcutaneous | 1 | (3) | 1 | (3) | 0 | (0) | 2 | (2) |
| tissue disorders | | | | | | | | |
| Alopecia | 0 | (0) | 1 | (3) | 0 | (0) | 1 | (1) |
| Hyperhidrosis | 1 | (3) | 0 | (0) | 0 | (0) | 1 | (1) |
| Immune system disorders | 0 | (0) | 1 | (3) | 0 | (0) | 1 | (1) |
| Smoke sensitivity | 0 | (0) | 1 | (3) | 0 | (0) | 1 | (1) |
| Metabolism and nutrition disorders | 0 | (0) | 1 | (3) | 0 | (0) | 1 | (1) |
| Hyperphagia | 0 | (0) | 1 | (3) | 0 | (0) | 1 | (1) |
| Increased appetite | 0 | (0) | 1 | (3) | 0 | (0) | 1 | (1) |
| Renal and urinary disorders | 1 | (3) | 0 | (0) | 0 | (0) | 1 | (1) |
| Haematuria | 1 | (3) | 0 | (0) | 0 | (0) | 1 | (1) |

In Table 22, adverse events were coded using MedDRA Dictionary v24.0. Frequencies are numbers of subjects experiencing at least one AE in that category. Subjects experiencing more than one adverse event in each category are counted only once for that category.

TABLE 23
Summary of Modified Observer's Assessment
of Alertness/Sedation (MOAA/S).

| | | 30 mg | 60 mg | Placebo |
|---|---|---|---|---|
| Time point | Statistic | (n = 33) | (n = 35) | (n = 34) |
| Baseline¹ | n | 33 | 35 | 33 |
| | Mean | 5.0 | 5.0 | 5.0 |
| | Median | 5.0 | 5.0 | 5.0 |
| | SD | 0.00 | 0.00 | 0.17 |
| | Min, Max | 5, 5 | 5, 5 | 4, 5 |
| 10 min post start | n | 10 | 11 | 11 |
| of infusion | Mean | 5.0 | 4.9 | 4.9 |
| | Median | 5.0 | 5.0 | 5.0 |
| | SD | 0.00 | 0.30 | 0.30 |
| | Min, Max | 5, 5 | 4, 5 | 4, 5 |
| Change from baseline | n | 10 | 11 | 11 |
| to 10 min post-start | Mean | 0.0 | −0.1 | 0.0 |
| | Median | 0.0 | 0.0 | 0.0 |
| | SD | 0.00 | 0.30 | 0.00 |
| | Min, Max | 0, 0 | −1, 0 | 0, 0 |
| 20 min post start | n | 33 | 35 | 33 |
| of infusion | Mean | 4.9 | 4.8 | 4.9 |
| | Median | 5.0 | 5.0 | 5.0 |
| | SD | 0.33 | 0.41 | 0.24 |
| | Min, Max | 4, 5 | 4, 5 | 4, 5 |
| Change from baseline | n | 33 | 35 | 32 |
| to 20 min post-start | Mean | −0.1 | −0.2 | −0.0 |
| | Median | 0.0 | 0.0 | 0.0 |
| | SD | 0.33 | 0.41 | 0.31 |
| | Min, Max | −1, 0 | −1, 0 | −11 |
| 30 min post start | n | 10 | 11 | 10 |
| of infusion | Mean | 4.9 | 4.9 | 4.8 |
| | Median | 5.0 | 5.0 | 5.0 |
| | SD | 0.32 | 0.30 | 0.42 |
| | Min, Max | 4, 5 | 4, 5 | 4, 5 |
| Change from baseline | n | 10 | 11 | 10 |
| to 30 min post-start | Mean | −0.1 | −0.1 | −0.1 |
| | Median | 0.0 | 0.0 | 0.0 |
| | SD | 0.32 | 0.30 | 0.57 |
| | Min, Max | −1, 0 | −1, 0 | −1, 1 |
| 40 min post start | n | 33 | 35 | 33 |
| of infusion | Mean | 4.8 | 4.8 | 4.8 |
| | Median | 5.0 | 5.0 | 5.0 |
| | SD | 0.44 | 0.43 | 0.50 |
| | Min, Max | 4, 5 | 4, 5 | 3, 5 |
| Change from baseline | n | 33 | 35 | 32 |
| to 40 min post-start | Mean | −0.2 | −0.2 | −0.2 |
| | Median | 0.0 | 0.0 | 0.0 |
| | SD | 0.44 | 0.43 | 0.55 |
| | Min, Max | −1, 0 | −1, 0 | −2, 1 |
| 50 min post start | n | 10 | 11 | 10 |
| of infusion | Mean | 4.9 | 4.9 | 4.6 |
| | Median | 5.0 | 5.0 | 5.0 |
| | SD | 0.32 | 0.30 | 0.52 |
| | Min, Max | 4, 5 | 4, 5 | 4, 5 |
| Change from baseline | n | 10 | 11 | 10 |
| to 50 min post-start | Mean | −0.1 | −0.1 | −0.3 |
| | Median | 0.0 | 0.0 | 0.0 |
| | SD | 0.32 | 0.30 | 0.67 |
| | Min, Max | −1, 0 | −1, 0 | −1, 1 |
| 1 hour post start | n | 33 | 35 | 33 |
| of infusion | Mean | 4.8 | 4.8 | 4.7 |
| | Median | 5.0 | 5.0 | 5.0 |
| | SD | 0.44 | 0.41 | 0.52 |
| | Min, Max | 4, 5 | 4, 5 | 3, 5 |
| Change from baseline | n | 33 | 35 | 32 |
| to 1 hour post-start | Mean | −0.2 | −0.2 | −0.3 |
| | Median | 0.0 | 0.0 | 0.0 |
| | SD | 0.44 | 0.41 | 0.57 |
| | Min, Max | −1, 0 | −1, 0 | −2, 1 |
| 1 hour 10 min post | n | 10 | 11 | 10 |
| start of infusion | Mean | 5.0 | 5.0 | 4.5 |
| | Median | 5.0 | 5.0 | 4.5 |
| | SD | 0.00 | 0.00 | 0.53 |
| | Min, Max | 5, 5 | 5, 5 | 4, 5 |
| Change from baseline to | n | 10 | 11 | 10 |
| 1 hour 10 min post-start | Mean | 0.0 | 0.0 | −0.4 |
| | Median | 0.0 | 0.0 | −0.5 |
| | SD | 0.00 | 0.00 | 0.70 |
| | Min, Max | 0, 0 | 0, 0 | −1, 1 |
| 1 hour 20 min post | n | 33 | 34 | 33 |
| start of infusion | Mean | 4.8 | 4.8 | 4.8 |
| | Median | 5.0 | 5.0 | 5.0 |
| | SD | 0.42 | 0.48 | 0.50 |
| | Min, Max | 4, 5 | 3, 5 | 3, 5 |
| Change from baseline to | n | 33 | 34 | 32 |
| 1 hour 20 min post-start | Mean | −0.2 | −0.2 | −0.2 |
| | Median | 0.0 | 0.0 | 0.0 |
| | SD | 0.42 | 0.48 | 0.55 |
| | Min, Max | −1, 0 | −2, 0 | −2, 1 |
| 1 hour 30 min post | n | 10 | 11 | 10 |
| start of infusion | Mean | 5.0 | 5.0 | 4.5 |
| | Median | 5.0 | 5.0 | 4.5 |
| | SD | 0.00 | 0.00 | 0.53 |
| | Min, Max | 5, 5 | 5, 5 | 4, 5 |
| Change from baseline to | n | 10 | 11 | 10 |
| 1 hour 30 min post-start | Mean | 0.0 | 0.0 | −0.4 |
| | Median | 0.0 | 0.0 | −0.5 |
| | SD | 0.00 | 0.00 | 0.70 |
| | Min, Max | 0, 0 | 0, 0 | −1, 1 |
| 1 hour 40 min post | n | 33 | 35 | 33 |
| start of infusion | Mean | 4.9 | 4.8 | 4.8 |
| | Median | 5.0 | 5.0 | 5.0 |
| | SD | 0.33 | 0.51 | 0.36 |
| | Min, Max | 4, 5 | 3, 5 | 4, 5 |
| Change from baseline to | n | 33 | 35 | 32 |
| 1 hour 40 min post-start | Mean | −0.1 | −0.2 | −0.1 |
| | Median | 0.0 | 0.0 | 0.0 |
| | SD | 0.33 | 0.51 | 0.42 |
| | Min, Max | −1, 0 | −2, 0 | −1, 1 |
| 1 hour 50 min post | n | 10 | 11 | 10 |
| start of infusion | Mean | 5.0 | 5.0 | 4.7 |
| | Median | 5.0 | 5.0 | 5.0 |
| | SD | 0.00 | 0.00 | 0.48 |
| | Min, Max | 5, 5 | 5, 5 | 4, 5 |
| Change from baseline to | n | 10 | 11 | 10 |
| 1 hour 50 min post-start | Mean | 0.0 | 0.0 | −0.2 |
| | Median | 0.0 | 0.0 | 0.0 |
| | SD | 0.00 | 0.00 | 0.63 |
| | Min, Max | 0, 0 | 0, 0 | −1, 1 |
| 2 hours | n | 33 | 35 | 33 |
| | Mean | 5.0 | 4.8 | 4.9 |
| | Median | 5.0 | 5.0 | 5.0 |
| | SD | 0.17 | 0.47 | 0.24 |
| | Min, Max | 4, 5 | 3, 5 | 4, 5 |
| Change from baseline | n | 33 | 35 | 32 |
| to 2 hours | Mean | −0.0 | −0.2 | −0.0 |
| | Median | 0.0 | 0.0 | 0.0 |
| | SD | 0.17 | 0.47 | 0.31 |
| | Min, Max | −1, 0 | −2, 0 | −1, 1 |
| 4 hours | n | 31 | 32 | 29 |
| | Mean | 5.0 | 4.9 | 4.9 |
| | Median | 5.0 | 5.0 | 5.0 |
| | SD | 0.00 | 0.30 | 0.26 |
| | Min, Max | 5, 5 | 4, 5 | 4, 5 |
| Change from baseline | n | 31 | 32 | 28 |
| to 4 hours | Mean | 0.0 | −0.1 | −0.0 |
| | Median | 0.0 | 0.0 | 0.0 |
| | SD | 0.00 | 0.30 | 0.33 |
| | Min, Max | 0, 0 | −1, 0 | −1, 1 |
| 6 hours | n | 31 | 32 | 29 |
| | Mean | 5.0 | 5.0 | 5.0 |
| | Median | 5.0 | 5.0 | 5.0 |
| | SD | 0.00 | 0.00 | 0.00 |
| | Min, Max | 5, 5 | 5, 5 | 5, 5 |
| Change from baseline | n | 31 | 32 | 28 |
| to 6 hours | Mean | 0.0 | 0.0 | 0.0 |
| | Median | 0.0 | 0.0 | 0.0 |
| | SD | 0.00 | 0.00 | 0.19 |
| | Min, Max | 0, 0 | 0, 0 | 0, 1 |

The MOAA/S scale used in Table 23 is a 6-point scale assessing responsiveness of patients.
Higher score indicates higher responsiveness.
¹Baseline value is defined as the last non-missing value prior to study drug administration.

TABLE 24
Summary of Clinician-Administered
Dissociative States Scale (CADSS).

| | | 30 mg | 60 mg | Placebo |
|---|---|---|---|---|
| Time point | Statistic | (n = 33) | (n = 35) | (n = 34) |
| Baseline¹ | n | 33 | 35 | 34 |
| | Mean | 0.0 | 0.3 | 0.0 |
| | Median | 0.0 | 0.0 | 0.0 |
| | SD | 0.17 | 1.23 | 0.17 |
| | Min, Max | 0, 1 | 0, 7 | 0, 1 |
| 40 min post infusion start | n | 33 | 35 | 32 |
| | Mean | 3.9 | 4.3 | 2.4 |
| | Median | 2.0 | 0.0 | 0.0 |
| | SD | 5.68 | 8.42 | 4.94 |
| | Min, Max | 0, 27 | 0, 32 | 0, 21 |
| Change from baseline to | n | 33 | 35 | 32 |
| 40 min post infusion start | Mean | 3.9 | 4.0 | 2.4 |
| | Median | 2.0 | 0.0 | 0.0 |
| | SD | 5.68 | 7.84 | 4.96 |
| | Min, Max | 0, 27 | −2, 27 | −1, 21 |
| 2 hours post infusion start | n | 33 | 35 | 33 |
| | Mean | 1.1 | 1.1 | 1.3 |
| | Median | 0.0 | 0.0 | 0.0 |
| | SD | 4.37 | 2.46 | 3.02 |
| | Min, Max | 0, 25 | 0, 8 | 0, 15 |
| Change from baseline to 2 | n | 33 | 35 | 33 |
| hours post infusion start | Mean | 1.1 | 0.8 | 1.3 |
| | Median | 0.0 | 0.0 | 0.0 |
| | SD | 4.38 | 2.29 | 3.02 |
| | Min, Max | −1, 25 | −2, 8 | 0, 15 |
| 4 hours post infusion start | n | 33 | 34 | 33 |
| | Mean | 0.4 | 0.3 | 0.4 |
| | Median | 0.0 | 0.0 | 0.0 |
| | SD | 1.92 | 0.67 | 1.20 |
| | Min, Max | 0, 11 | 0, 2 | 0, 6 |
| Change from baseline to 4 | n | 33 | 34 | 33 |
| hours post infusion start | Mean | 0.4 | −0.0 | 0.4 |
| | Median | 0.0 | 0.0 | 0.0 |
| | SD | 1.93 | 1.14 | 1.17 |
| | Min, Max | −1, 11 | −5, 2 | 0, 6 |
| 6 hours post infusion start | n | 10 | 11 | 10 |
| | Mean | 0.0 | 0.0 | 0.0 |
| | Median | 0.0 | 0.0 | 0.0 |
| | SD | 0.00 | 0.00 | 0.00 |
| | Min, Max | 0, 0 | 0, 0 | 0, 0 |
| Change from baseline to 6 hours | n | 10 | 11 | 10 |
| | Mean | −0.1 | 0.0 | 0.0 |
| | Median | 0.0 | 0.0 | 0.0 |
| | SD | 0.32 | 0.00 | 0.00 |
| | Min, Max | −1, 0 | 0, 0 | 0, 0 |

In Table 24, the CADSS assesses the alertness or dissociative state of subjects administered PCN 101.
Higher score indicates increases severity.
¹Baseline value is defined as the last non-missing value prior to study drug administration.

### Example 2: Pharmacokinetics (PK) of R(−)-ketamine in Human Subjects

This was a Phase 1 randomized, placebo-controlled, double-blind, single dose study conducted at one center. One part of this study was an ascending dose, randomized, placebo-controlled, double-blind safety and tolerability study of single dose intravenous (IV) infusions of R(−)-ketamine, or placebo (saline) administered over 40 minutes to healthy subjects. A total of up to 48 healthy subjects were enrolled into 6 sequential cohorts of 8 subjects each. The pharmacokinetic (PK) population was 36 subjects, with 12 additional subjects receiving placebo. All subjects who received all or part of the infusion of the study drug and had a sufficient evaluable concentration-time data to allow determination of at least one PK parameter, among Cₘₐₓ, Tₘₐₓ or AUC₀₋ₜ were included in the PK Population. Subjects were male or female healthy subjects, 18 to 65 years of age. Within each cohort, subjects were randomized in a 3:1 ratio between active drug and placebo; 6 subjects were administered R(−)-ketamine and 2 were administered placebo. Each cohort started with sentinel dosing of 2 subjects, randomized 1:1 to either a R(−)-ketamine cohort dose or placebo cohort dose with a safety review performed prior to enrolling the full cohort. Cohort 1 was dosed at 5 mg, followed by Cohort 2 at 15 mg, Cohort 3 at 30 mg, Cohort 4 at 60 mg, Cohort 5 at 100 mg, and Cohort 6 at 150 mg R(−)-ketamine. Dose escalation was continued until either an acceptable tolerated dose (ATD) was identified or the 150 mg cohort was completed. In addition to the safety and tolerability data, the study also evaluated the dose-related PK of each 40-minute IV infusion of R(−)-ketamine.

To evaluate the pharmacokinetics of R(−)-ketamine, blood was drawn from subjects for PK analysis at the following sampling times: pre-dose (−30 minutes); and from the start of infusion (5, 10, and 15 minutes [±1 minute], 30 minutes [±5 minutes], 40 minutes [immediately prior to infusion completion], 1, 1.5, 2, and 3 hours [±5 minutes], 4 and 8 hours [±10 minutes], 12 and 24 hours [±15 minutes]).

Plasma PK parameters of R(−)-ketamine and its metabolites (norketamine, 6-hydroxynorketamine, and dehydronorketamine) evaluated on include the following:
Maximum plasma concentration (Cmax),
Time to Cmax (Tmax),
Area under the plasma concentration curve (AUC₀₋ₜ, AUC_{0-∞})
Elimination rate constant (λz),
Half-live (t½)
Clearance (CL, P R(−)-ketamine only),
Volume of distribution (Vz, R(−)-ketamine only), and
Metabolite to parent ratios (area under curve [AUC] and Cₘₐₓ).

The dose proportionality was assessed both visually and statistically using calculated Cmax and AUC values. Statistical analysis was carried out according to the statistical analysis plan. Continuous variables: Descriptive statistics included the number of non-missing values (n), arithmetic mean, standard deviation (SD), median, minimum, and maximum values. The minimum and maximum values are displayed to the same decimal precision as the source data, the arithmetic mean, SD, and median values are displayed to one more decimal than the source data for the specific variable. The appropriate precision for derived variables is determined based on the precision of the data on which the derivations are based, and statistics are presented in accordance with the abovementioned rules. For PK concentration data, the number of non-missing values, number of below limit of quantification (BLQ) values, arithmetic mean, SD, median, minimum, maximum, coefficient of variation (CV %), geometric mean, and geometric CV (geo CV %) values are presented. For PK parameter data, the number of non-missing values, arithmetic mean, SD, median, minimum, maximum, CV %, geometric mean, and geometric CV (geo CV %) values are presented.

Intravenous administration of allocated blinded study drug (Morning, Day 1) was infused over 40 minutes. Blood collection for plasma PK analysis occurred at infusion 5, 10, 15, 30, 40, 60, and 90 minutes, and 2, 3, 4, 8, and 12 hours after the start of infusion.

Table 25 provides the doses administered to each cohort.

TABLE 25
Dose Administration

| cohort | Treatment dose | route | No. Doses |
|---|---|---|---|
| 1 | 5 mg R(−)-ketamine or placebo | I.V. | 1 |
| 2 | 15 mg R(−)-ketamine or placebo | I.V. | 1 |
| 3 | 30 mg R(−)-ketamine or placebo | I.V. | 1 |
| 4 | 60 mg R(−)-ketamine or placebo | I.V. | 1 |
| 5 | 100 mg R(−)-ketamine or placebo | I.V. | 1 |
| 6 | 150 mg R(−)-ketamine or placebo | I.V. | 1 |

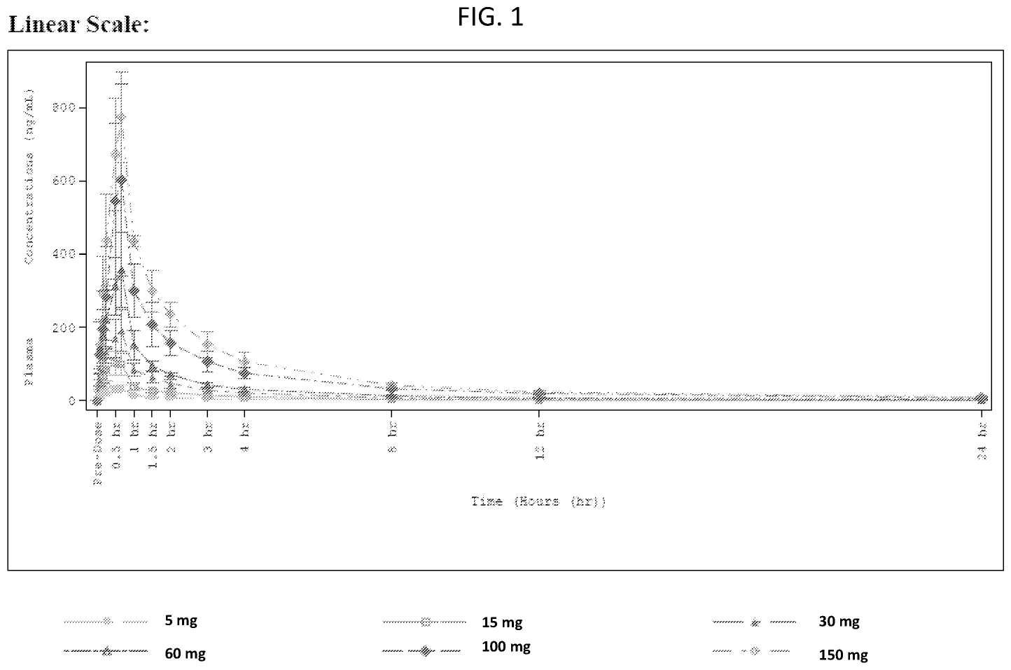

Plasma concentrations of R(−)-ketamine and its metabolites norketamine, 6-hydroxynorketamine, and dehydronorketamine were determined using a suitable validated analytical method with a lower limit of quantitation (LOQ) of 1 ng/mL. The results are shown in FIGS. 1-8.

R(−)-ketamine

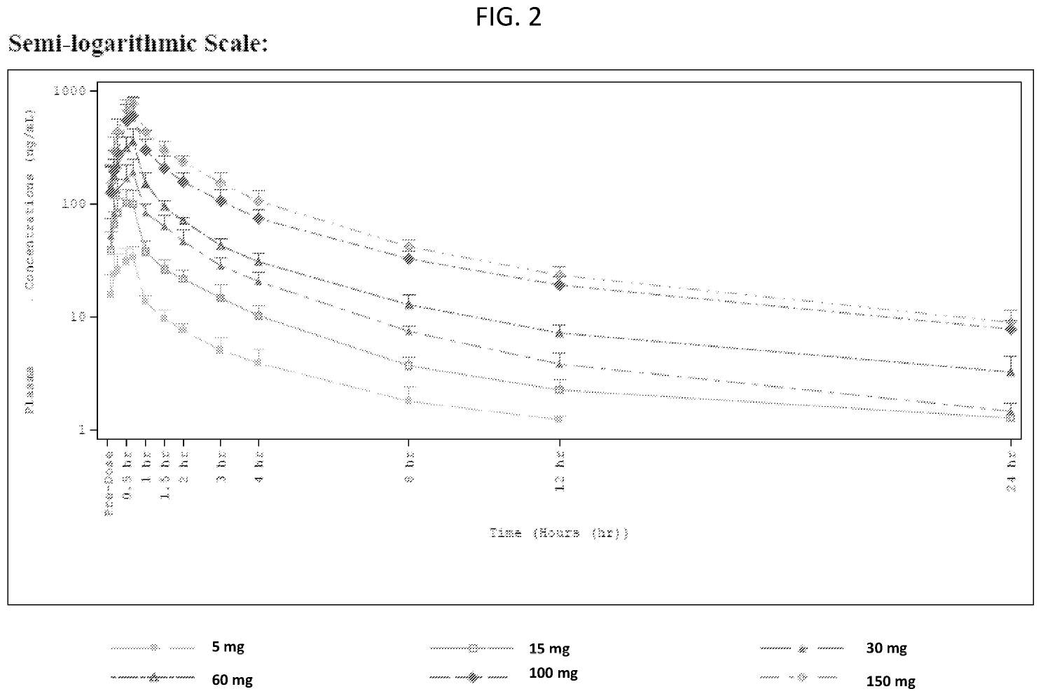

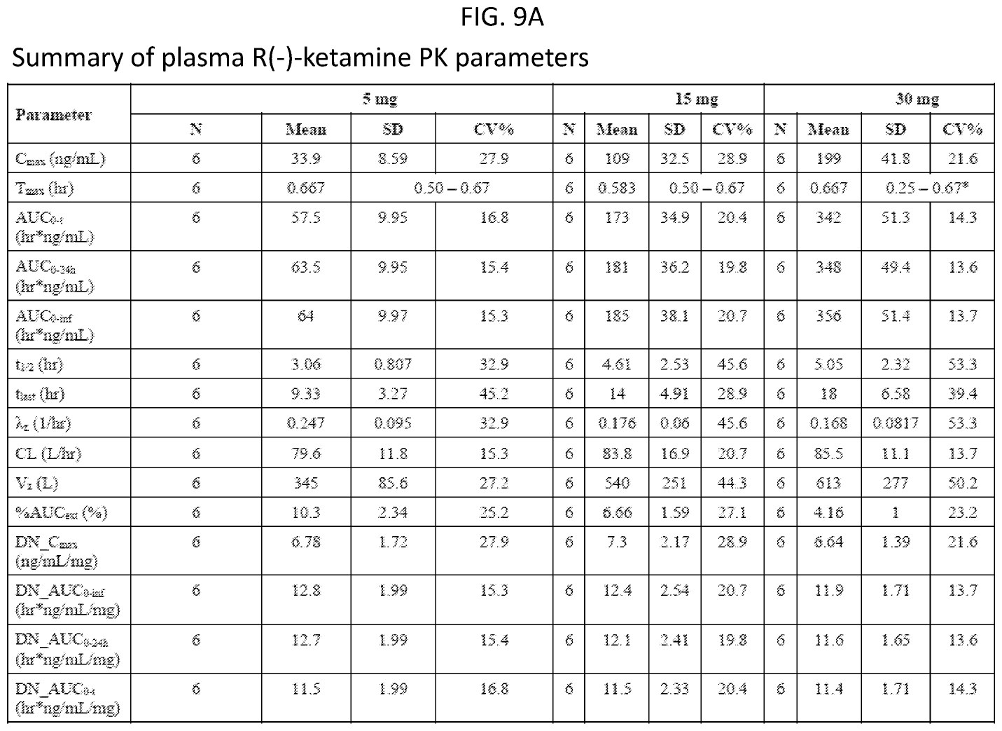

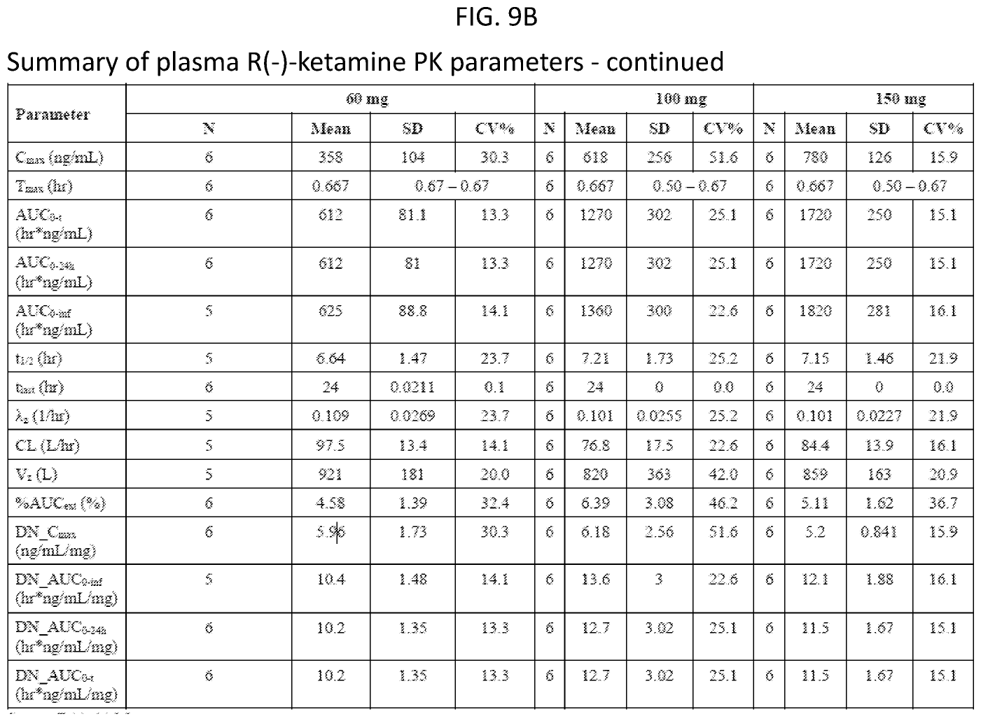

Plasma concentrations of R(−)-ketamine are shown in FIGS. 1-2. Selected PK parameters of R(−)-ketamine by cohort are presented in the table shown in FIGS. 9A-9B. Analysis of plasma concentrations following the IV infusion administration (40 minutes duration) of R(−)-ketamine at 5 mg, 15 mg, 30 mg, 60 mg, 100 mg, or 150 mg demonstrated that, regardless of dose level, R(−)-ketamine concentrations were quantifiable in all subjects at the earliest timepoint (5 minutes) with median plasma Tmax of 40 minutes (range: 15 minutes to 40 minutes). Overall, across all the cohorts, the plasma exposure of R(−)-ketamine as indicated by Cmax and AUC (AUC0-t, AUC0-24h, AUC0-inf) increased with the increase in dose administered.

Arithmetic mean Cmax (CV %) increased from 33.9 ng/ml (27.9%) following the IV administration of 5 mg, up to 780 ng/ml (15.9%) following the IV administration of 150 mg representing a 23-fold increase in Cmax for a 30-fold increase in dose. Arithmetic mean AUC0-24h (CV %) increased from 63.5 hr*ng/mL (15.4%) following the IV administration of 5 mg R(−)-ketamine to 1720 hr*ng/mL (15.1%) following the IV administration of 150 mg R(−)-ketamine representing a 27.1 fold increase in AUC0-24h for a 30-fold increase in dose. Arithmetic mean AUC0-t (CV %) increased from 57.5 hr*ng/ml (16.8%) following the IV administration of 5 mg R(−)-ketamine, to 1720 hr*ng/ml (15.1%) following IV administration of 150 mg R(−)-ketamine, representing a 29.9 fold increase in AUC0-t for a 30 fold increase in dose. Arithmetic mean AUC0-inf (CV %) increased from 64.0 hr*ng/ml (15.3%) following the IV administration of 5 mg R(−)-ketamine, to 1820 hr*ng/ml (16.1%) following the IV administration of 150 mg R(−)-ketamine, representing a 28.4-fold increase in AUC0-inf for a 30-fold increase in dose. Assessment of dose proportionality of R(−)-ketamine from 5 mg to 150 mg using the power model and graphical evaluation revealed that R(−)-ketamine pharmacokinetics increases in an approximately dose proportional manner for Cmax, AUC0-t, AUC0-24h, and AUC0-inf. Overall, inter-individual variability of R(−)-ketamine was low to moderate for Cmax, AUC0-t, AUC0-24h, and AUC0-inf, with CV % values ranging from 15.9% to 51.6%, 13.3% to 25.1%, 13.3% to 25.1%, and 13.7% to 22.6%, respectively. Following attainment of peak R(−)-ketamine plasma concentrations after IV administration, a monophasic decline was observed with arithmetic mean t½ range (CV %) of 3.06 to 7.21 hours (21.9 to 53.3%) across all dose cohorts from 5 mg to 150 mg. Overall, arithmetic mean (CV %) CL and Vz of plasma R(−)-ketamine were approximately consistent across the 5 mg to 150 mg R(−)-ketamine cohorts in the range of 76.8 to 97.5 L/hr (13.7% to 22.6%) and 345 to 921 L (20.0% to 50.2%), respectively.

### Norketamine

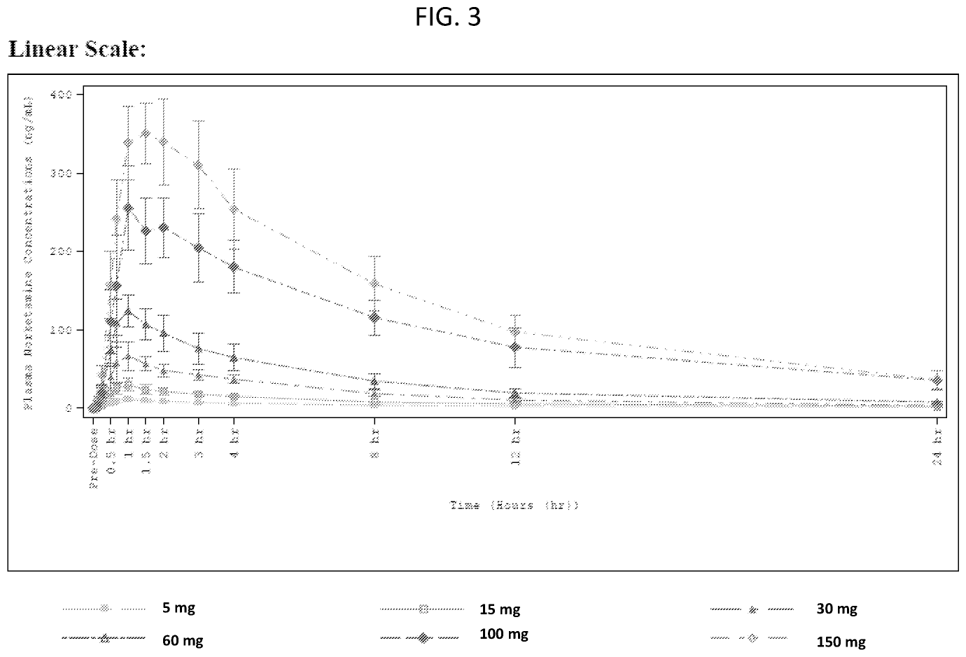

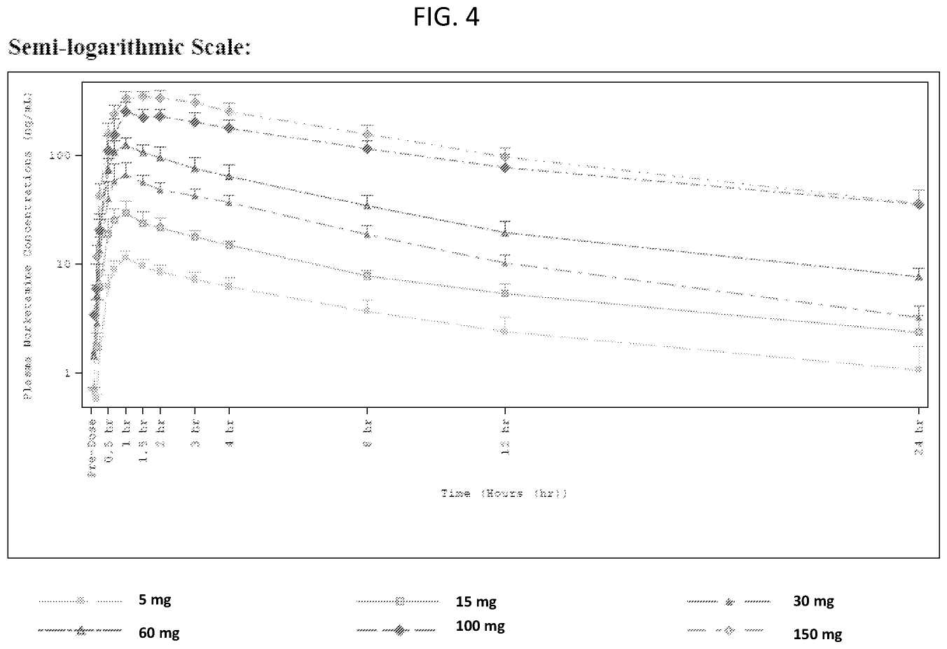

Plasma concentrations of norketamine are shown in FIGS. 3-4. Selected norketamine

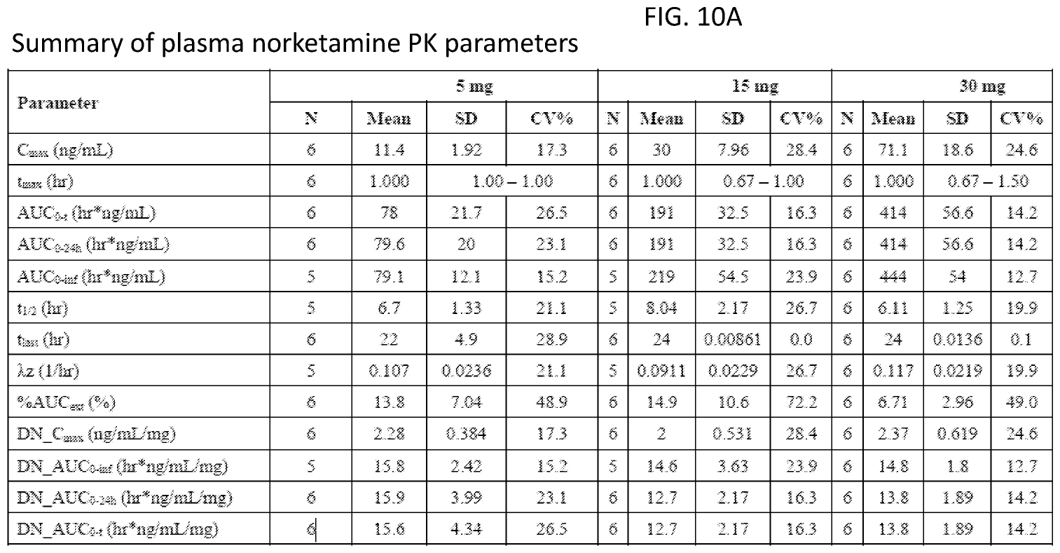

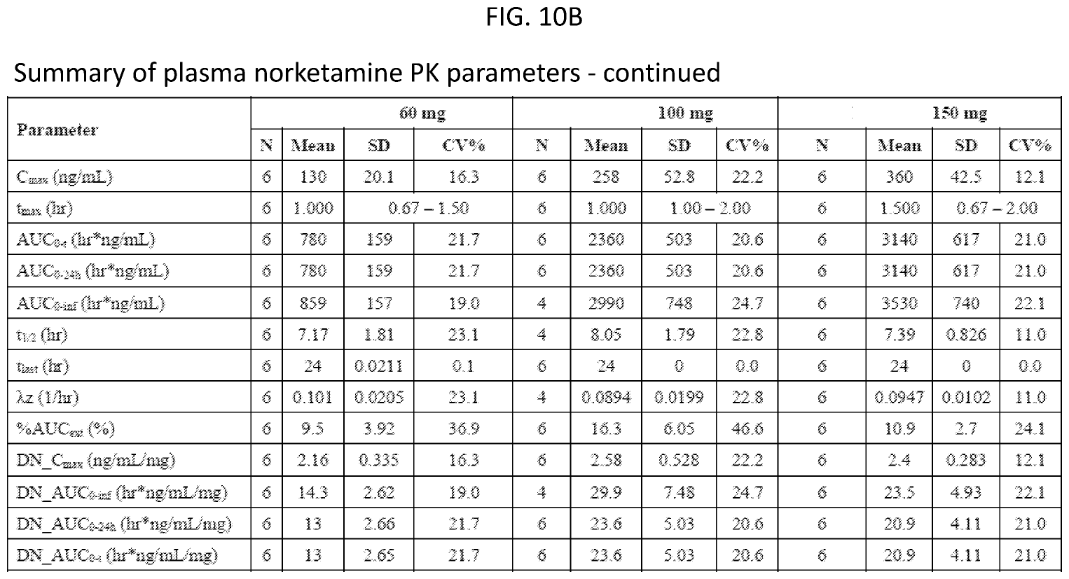

PK parameters of R(−)-ketamine cohorts, by cohort, are presented in the table shown in FIGS. 10A-10B. Analysis of plasma concentrations of norketamine following the IV infusion (40 minutes duration) of R(−)-ketamine at 5 mg, 15 mg, 30 mg, 60 mg, 100 mg or 150 mg demonstrated that, regardless of dose level of R(−)-ketamine, norketamine concentrations were quantifiable in all subjects at the earliest timepoints (10 minutes to 15 minutes) with approximate median plasma Tₘₐₓ of 1 hour (Range: 40 minutes to 2.00 hour). Overall, across all the cohorts, the plasma exposure of norketamine as indicated by Cₘₐₓ and AUC (AUC₀₋ₜ, AUC_{0-24 h}, AUC_{0-inf}) increased with increasing R(−)-ketamine dose.

Arithmetic mean Cₘₐₓ (CV %) of norketamine increased from 11.4 ng/ml (17.3%) following the IV administration of 5 mg R(−)-ketamine, up to 360 ng/mL (12.1%) following the IV administration of 150 mg R(−)-ketamine representing a 31.6-fold increase in Cmax of norketamine for a 30.0-fold increase in R(−)-ketamine dose. Arithmetic mean AUC_{0-24 h} (CV %) of norketamine increased from 79.6 hr*ng/ml (23.1%) following the IV administration of 5 mg R(−)-ketamine to 3140 hr*ng/ml (21.0%) following the IV administration of 150 mg R(−)-ketamine representing a 39.4-fold increase in AUC_{0-24 h} of norketamine for a 30-fold increase in R(−)-ketamine dose. Arithmetic mean AUC₀₋ₜ (CV %) of norketamine increased from 78.0 hr*ng/ml (26.5%) following the IV administration of 5 mg to 3140 hr*ng/ml (21.0%) following the IV administration of 150 mg representing a 40.3 fold increase in AUC₀₋ₜ for a 30-fold increase in dose. Arithmetic mean AUC_{0-inf} (CV %) of norketamine increased from 79.1 hr*ng/ml (15.2%) following the IV administration of 5 mg to 3530 hr*ng/ml (22.1%) following the IV administration of 150 mg representing a 44.6-fold increase in AUC_{0-inf} for a 30-fold increase in dose. Assessment of dose proportionality of norketamine from 5 mg to 150 mg R(−)-ketamine using the power model and graphical evaluation revealed that norketamine pharmacokinetics increased in an approximately dose proportional manner for Cₘₐₓ, AUC₀₋ₜ, AUC_{0-24 h}, and AUC_{0-inf}. Overall, inter-individual variability of norketamine was low to moderate for Cₘₐₓ, AUC₀₋ₜ, AUC_{0-24 h}, and AUC_{0-inf}, with CV % values ranging from 12.1% to 28.4%, 14.2% to 26.5%, 14.2% to 23.1%, and 12.7% to 24.7%, respectively. Following the attainment of peak plasma concentrations, a monophasic decline of norketamine was observed with arithmetic mean t_{1/2} range (CV %) of 6.70 to 8.05 hours (11.0% to 26.7%) across all dose cohorts from 5 mg to 150 mg R(−)-ketamine.

6-hydroxynorketamine

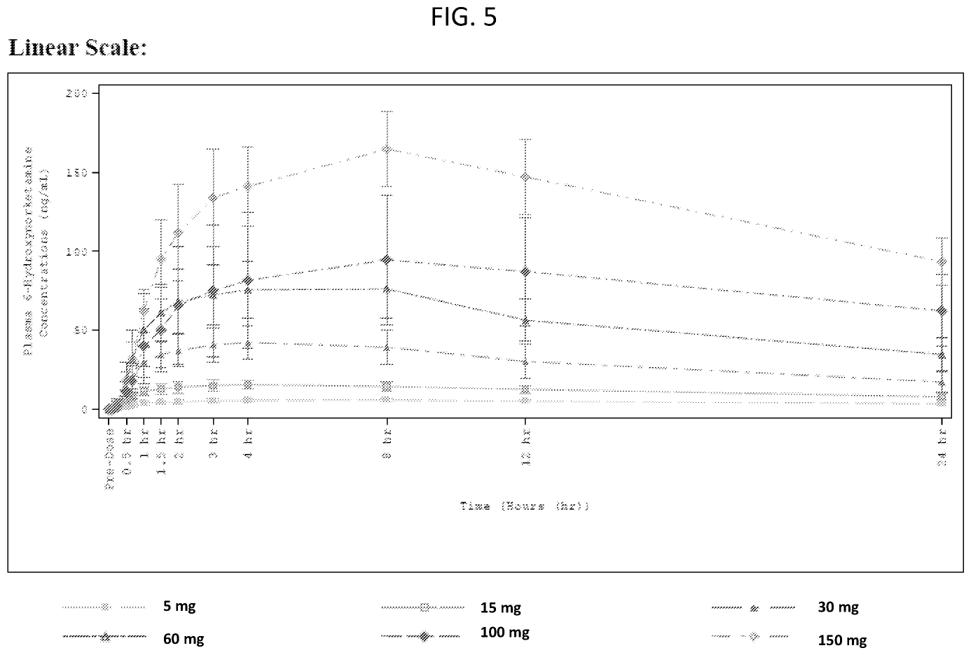

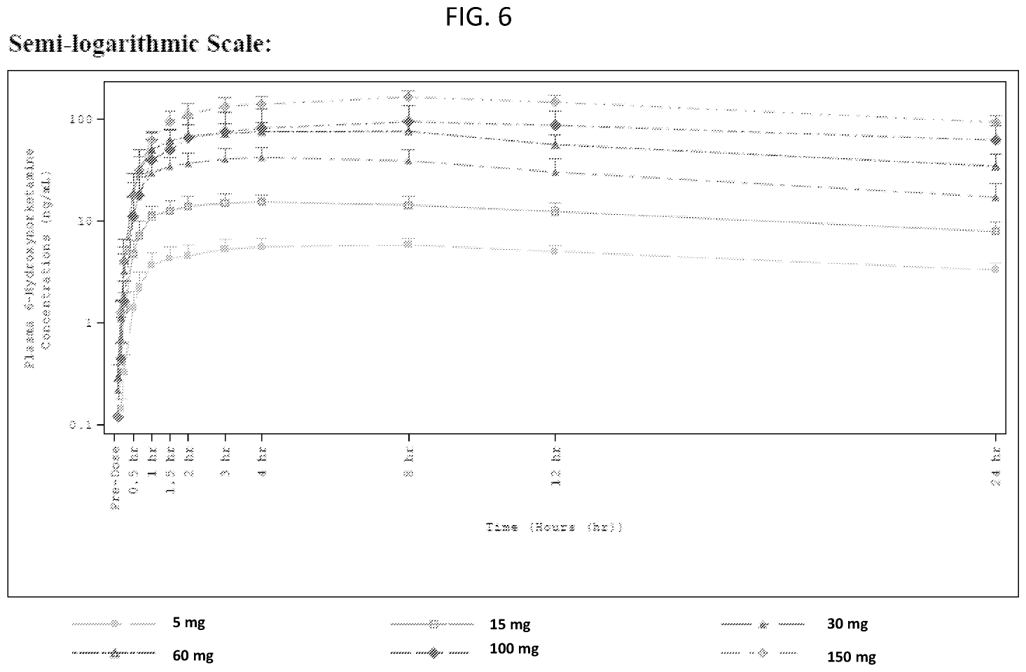

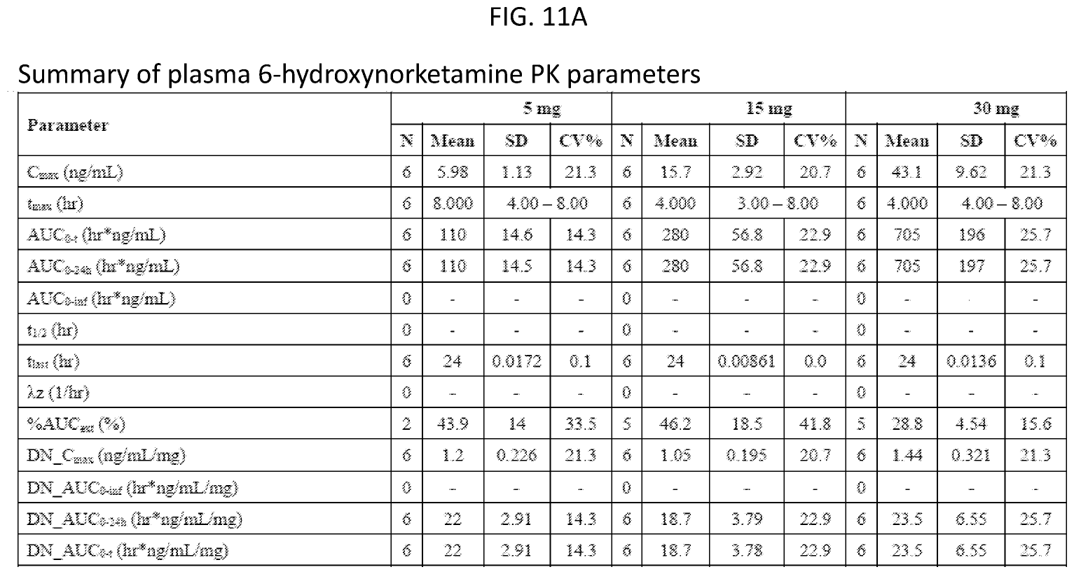

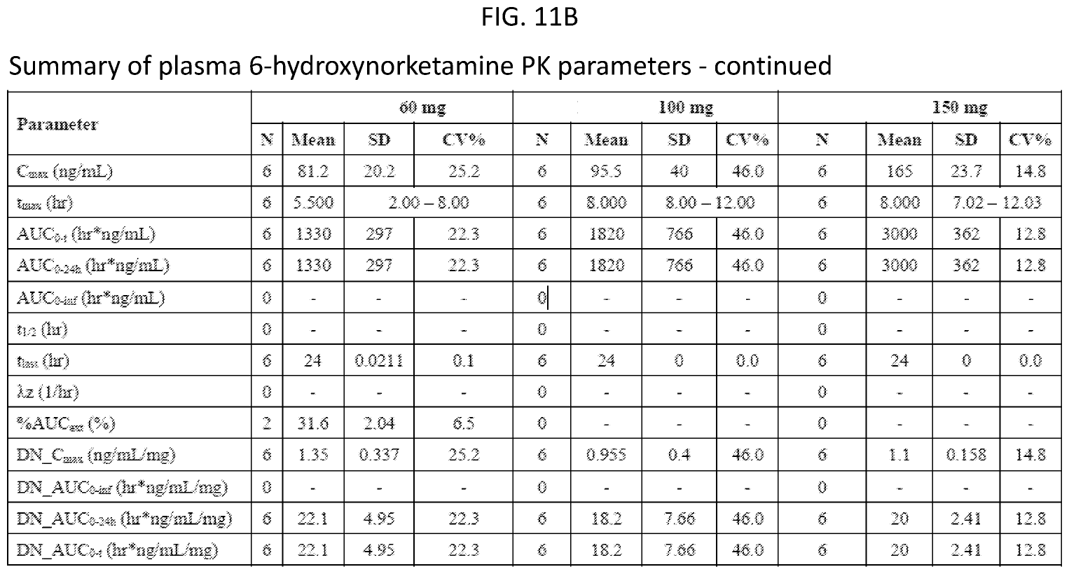

Plasma concentrations of 6-hydroxynorketamine are shown in FIGS. 5-6. Selected PK parameters for 6-hydroxynorketamine of R(−)-ketamine cohorts, by cohort, are presented in the table shown in FIGS. 11A-11B. Analysis of plasma concentrations following the IV infusion administration (40 minutes duration) of R(−)-ketamine at 5 mg, 15 mg, 30 mg, 60 mg, 100 mg or 150 mg demonstrated that, regardless of dose level, 6-hydroxynorketamine concentrations were quantifiable in all subjects at the earliest timepoints (10 minutes to 15 minutes) with approximate median plasma Tₘₐₓ range of 4.00 to 8.00 hours. Overall, across all the cohorts, the plasma exposure of 6-hydroxynorketamine as indicated by Cₘₐₓ and AUC (AUC₀₋ₜ and AUC_{0-24 h}) increased with R(−)-ketamine dose.

Arithmetic mean Cₘₐₓ (CV %) of 6-hydroxynorketamine increased from 5.98 ng/ml (21.3%) following the IV administration of 5 mg R(−)-ketamine, up to 165 ng/ml (14.8%) following the IV administration of 150 mg R(−)-ketamine representing a 27.6-fold increase in Cₘₐₓ for 6-hydroxynorketamine with a 30.0-fold increase in R(−)-ketamine dose. Arithmetic mean AUC_{0-24 h} and AUC0-t (CV %) of 6-hydroxynorketamine increased from 110 hr*ng/ml (14.3%) following the IV administration of 5 mg R(−)-ketamine to 3000 hr*ng/ml (12.8%) following the IV administration of 150 mg R(−)-ketamine representing a 27.3-fold increase in AUC_{0-24 h} and AUC₀₋ₜ for 6-hydroxynorketamine with a 30-fold increase in R(−)-ketamine dose. Assessment of dose proportionality of 6-hydroxynorketamine from 5 mg to 150 mg R(−)-ketamine using the power model and graphical evaluation revealed that 6-hydroxynorketamine pharmacokinetics increases in an approximately dose proportional manner for Cₘₐₓ, AUC₀₋ₜ, and AUC_{0-24 h}. Overall, inter-individual variability of 6-hydroxynorketamine was low to moderate for Cₘₐₓ and AUC₀₋ₜ, or AUC_{0-24 h} with CV % values ranging from 14.8% to 46.0%, and 12.8% to 46.0%, respectively.

### Dehydronorketamine

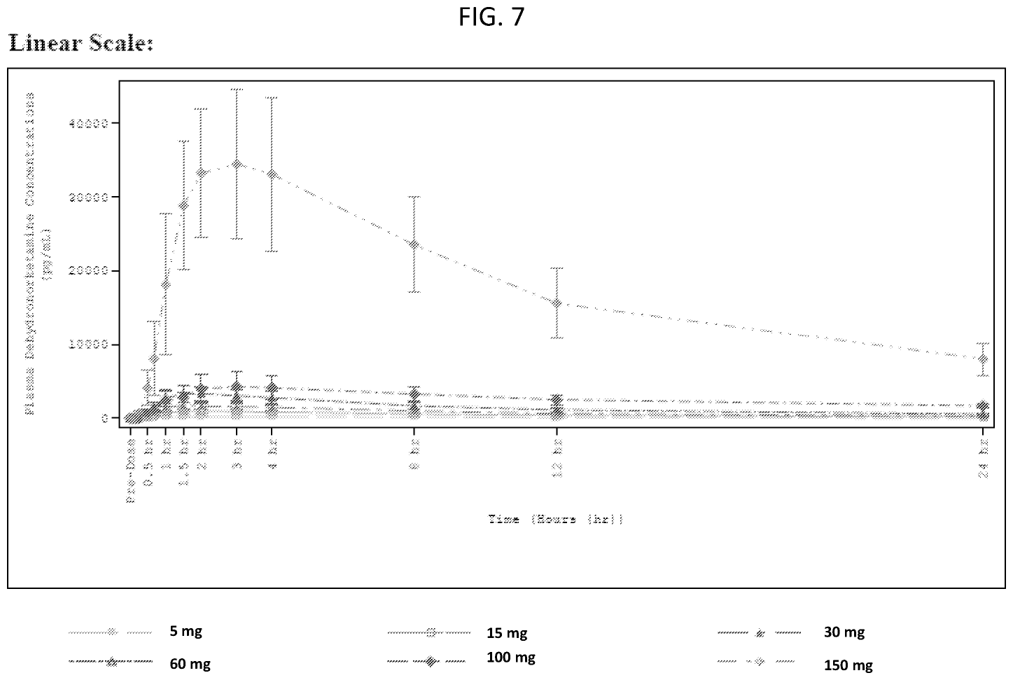

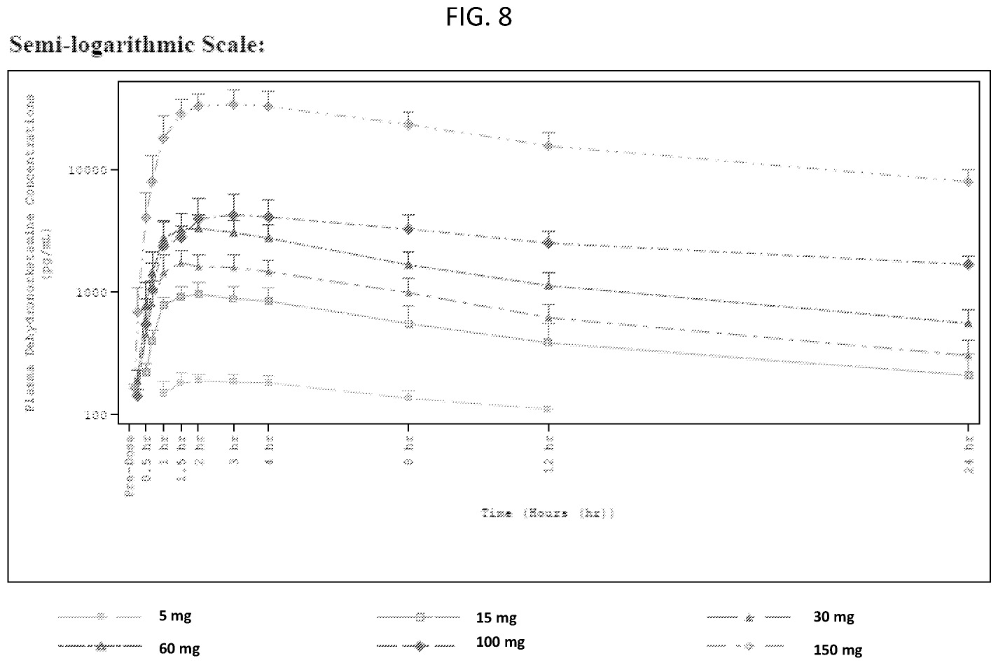

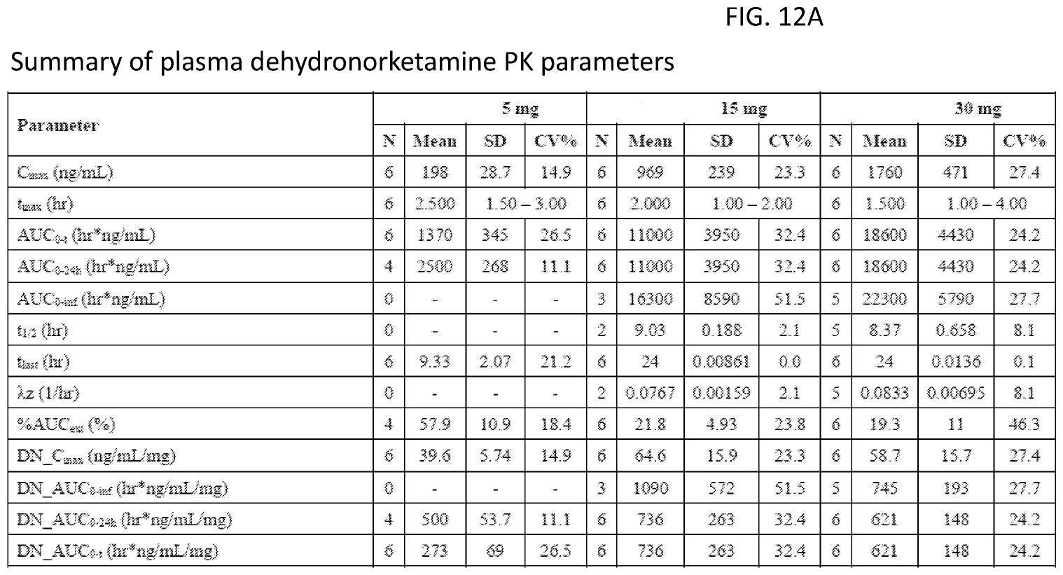

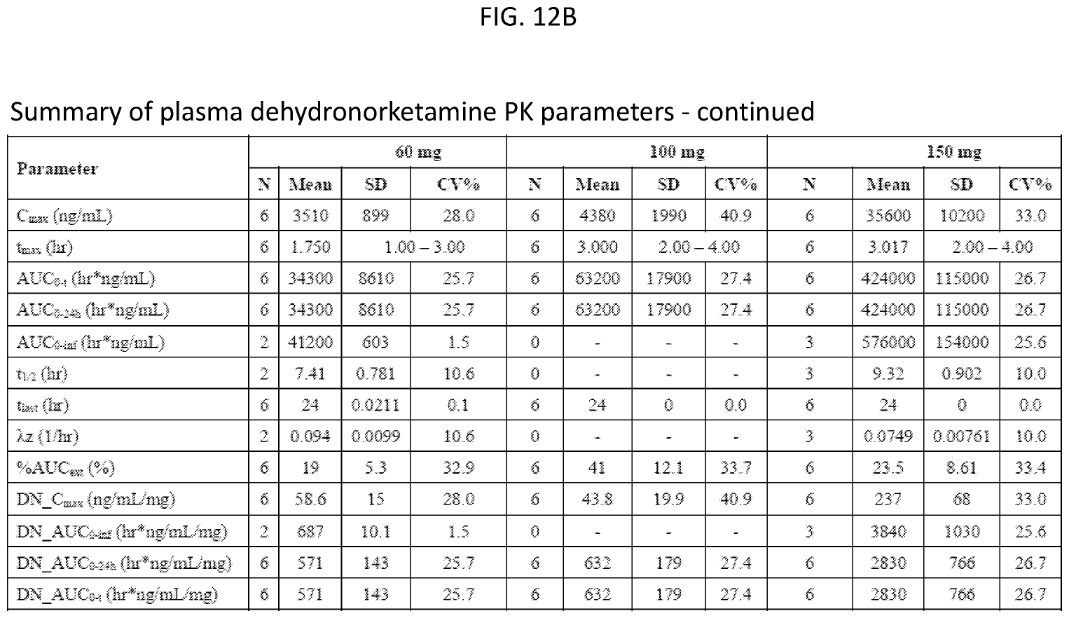

Plasma concentrations of dehydronorketamine are shown in FIGS. 7-8. Selected PK parameters for dehydronorketamine of R(−)-ketamine cohorts, by cohort, are presented in the table shown in FIGS. 12A-12B. Analysis of plasma concentrations following the IV infusion administration (40 minutes duration) of R(−)-ketamine at doses of 5 mg, 15 mg, 30 mg, 60 mg, 100 mg or 150 mg demonstrated that, regardless of dose level, dehydronorketamine concentrations were quantifiable in all subjects at the earliest timepoints (15 minutes to 1.50 hours), with approximate median plasma Tₘₐₓ range of 1.50 to 3.02 hours. Overall, across all the cohorts, the plasma exposure of dehydronorketamine as indicated by Cₘₐₓ and AUC (AUC0-t, AUC_{0-24 h}, AUC_{0-inf}) increased with increasing R(−)-ketamine dose.

The arithmetic mean Cmax (CV %) of dehydronorketamine increased from 198 pg/mL (14.9%) following the IV administration of 5 mg R(−)-ketamine, up to 35600 pg/mL (33.0%) following the IV administration of 150 mg R(−)-ketamine representing a 179.8-fold increase in Cₘₐₓ for dehydronorketamine with a 30-fold increase in R(−)-ketamine dose. Arithmetic mean AUC_{0-24 h} (CV %) of dehydronorketamine increased from 2500 hr*pg/mL (11.1%) following the IV administration of 5 mg R(−)-ketamine to 424000 hr*pg/mL (26.7%) following the IV administration of 150 mg R(−)-ketamine representing a 169.6-fold increase in AUC_{0-24 h} for dehydronorketamine with a 30-fold increase in R(−)-ketamine dose.

The arithmetic mean AUC₀₋ₜ (CV %) of dehydronorketamine increased from 1370 hr*pg/mL (26.5%) following the IV administration of 5 mg to 424000 hr*pg/mL (26.7%) following the IV administration of 150 mg R(−)-ketamine representing a 309.5-fold increase in AUC₀₋ₜ for dehydronorketamine with a 30-fold increase in R(−)-ketamine dose. The arithmetic mean AUC_{0-inf} (CV %) of dehydronorketamine increased from 16300 hr*pg/mL (51.5%) following the IV administration of 15 mg R(−)-ketamine, to 576000 hr*pg/mL (25.6%) following the IV administration of 150 mg R(−)-ketamine representing a 35.3-fold increase in AUC_{0-inf} for dehydronorketamine with a 10-fold increase in R(−)-ketamine dose.

Assessment of dose proportionality of dehydronorketamine from 5 mg to 150 mg R(−)-ketamine using the power model and graphical evaluation revealed that dehydronorketamine pharmacokinetics increases in an approximately higher dose proportional manner for Cₘₐₓ, AUC₀₋ₜ, AUC_{0-24 h}, and AUC_{0-inf}. Overall, inter-individual variability of dehydronorketamine was low to moderate for Cₘₐₓ, AUC0-t, AUC_{0-24 h}, and AUC_{0-inf} with CV % values ranging from 14.9% to 40.9%, 24.2% to 32.4%, 11.1% to 32.4%, and 1.5% to 51.5%, respectively. Following attainment of peak plasma concentrations, the arithmetic mean t_{1/2} range (CV %) of dehydronorketamine was 7.41 to 9.32 hours (2.1% to 10.6%) across all dose cohorts from 15 mg to 60 mg and 150 mg.

### PK Parameters for Metabolite to Parent (M/P) Ratio

The overall arithmetic mean (geometric CV %) ratio of norketamine/R(−)-ketamine based on Cmax, AUC0-t, AUC0-24h, and AUC0-inf across all dose cohorts from 5 mg to 150 mg R(−)-ketamine were in the range of 0.264 to 0.448 (21.7% to 32.8%), 1.05 to 1.76 (8.7% to 33.0%), 1.00 to 1.76 (8.7% to 33.0%), and 1.14 to 1.88 (11.5% to 28.0%), respectively. The overall arithmetic mean (geometric CV %) ratio of 6-hydroxynorketamine/R(−)-ketamine based on Cmax, AUC0-t, and AUC0-24h across all dose cohorts from 5 mg to 150 mg R(−)-ketamine were in the range of 0.151 to 0.251 (22.5% to 90.0%), 1.54 to 2.22 (23.8% to 63.3%), 1.54 to 2.22 (23.8% to 63.3%), respectively. The overall arithmetic mean (geometric CV %) ratio of dehydronorketamine/R(−)-ketamine based on Cmax, AUC0-t, AUC0-24h, and AUC0-inf across all dose cohorts from 5 mg to 150 mg R(−)-ketamine were in the range of 0.00581 to 0.0437 (19.3% to 56.8%), 0.0223 to 0.233 (24.5% to 35.0%), 0.0512 to 0.233 (17.0% to 33.1%), and 0.0585 to 0.27 (17.0% to 39.7%), respectively.

### Summary of Dose Proportionality of Plasma PK Parameters

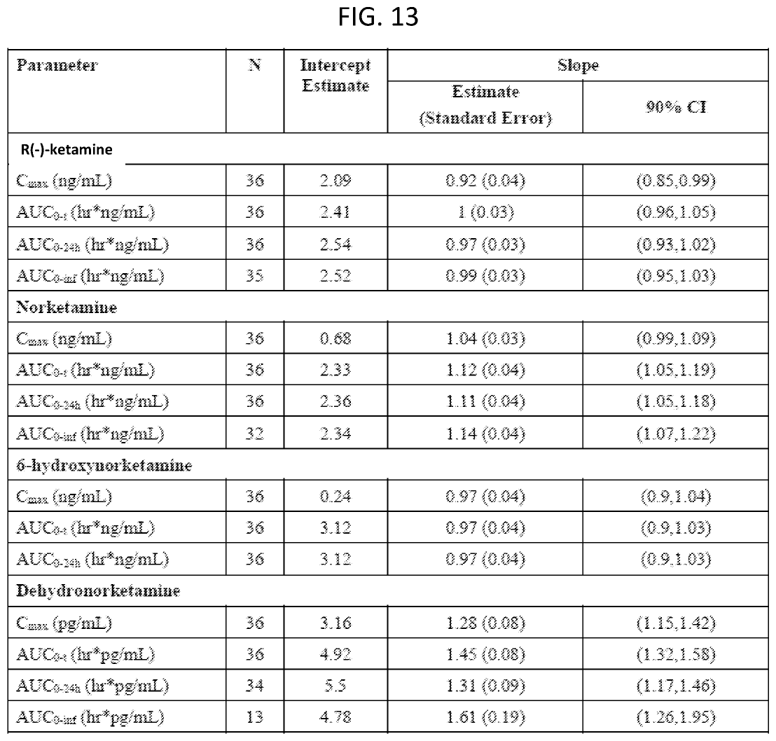

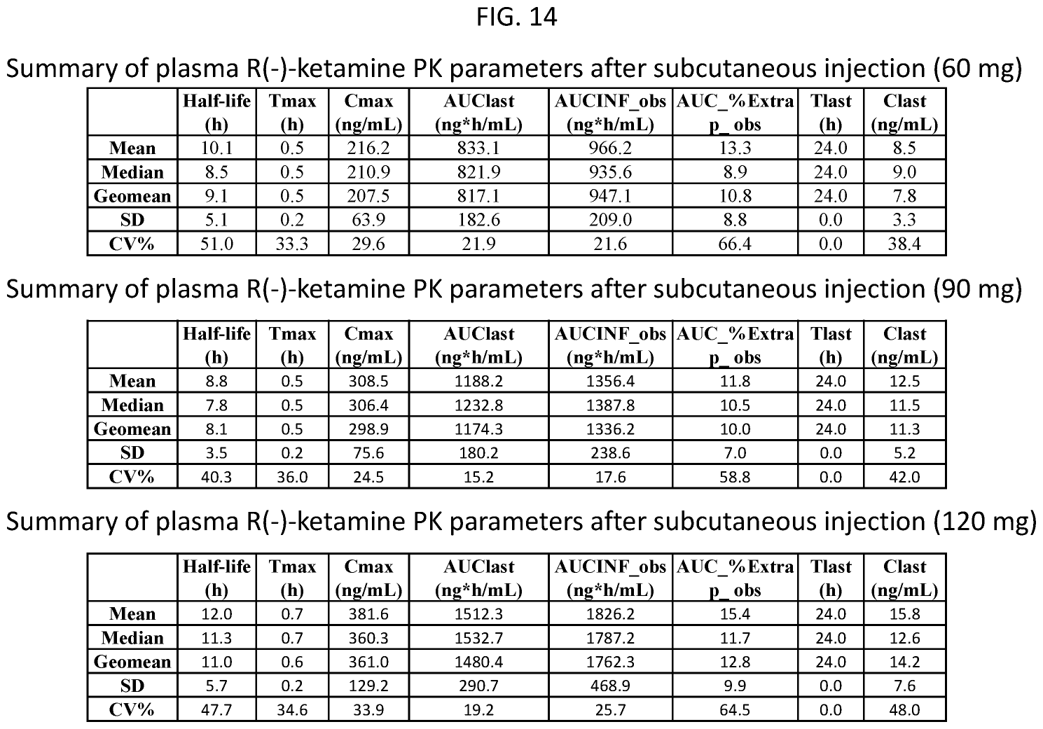

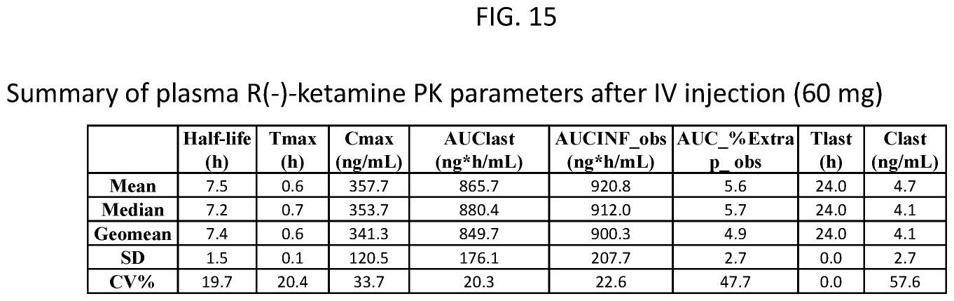

The linearity of the increase in exposure with respect to the increasing dose administered was evaluated for Cₘₐₓ and AUC for R(−)-ketamine and its metabolites (norketamine, 6-hydroxynorketamine and dehydronorketamine). Dose proportionality as measured by Cₘₐₓ, AUC₀₋ₜ, AUC_{0-24 h}, and AUC_{0-inf} was assessed using the power model (with natural log-transformed PK parameter values and natural log-transformed dose) and graphical evaluation (using scatter and power model regression plots of dose normalized PK parameter values and treatment doses). The power model was used to estimate the slope parameter and the 90% confidence intervals for the slope. A summary of dose proportionality assessment for R(−)-ketamine, norketamine, 6-hydroxynorketamine, and dehydronorketamine is presented in the table shown in FIG. 13.

Across the dose range of R(−)-ketamine (5 mg, 15 mg, 30 mg, 60 mg, 100 mg, and 150 mg), the increase in exposure of R(−)-ketamine was dose proportional for Cₘₐₓ, AUC₀₋ₜ, AUC_{0-24 h}, and AUC_{0-inf} with slope estimates (B1) and 90% CI of 0.92 (0.85 to 0.99), 1.00 (0.96 to 1.05), 0.97 (0.93 to 1.02), and 0.99 (0.95 to 1.03), respectively. The graphical evaluation also suggests the exposure of R(−)-ketamine (Cₘₐₓ, AUC₀₋ₜ, AUC_{0-24 h}, and AUC_{0-inf}) across the dose range was dose proportional.

Across the dose range of R(−)-ketamine (5 mg, 15 mg, 30 mg, 60 mg, 100 mg, and 150 mg), the increase in exposure of norketamine was dose proportional for Cₘₐₓ, AUC₀₋ₜ, AUC_{0-24 h}, and AUC_{0-inf} with slope estimates (1) and 90% CI of 1.04 (0.99 to 1.09), 1.12 (1.05 to 1.19), 1.11 (1.05 to 1.18), and 1.14 (1.07 to 1.22), respectively. The graphical evaluation also suggests the exposure of norketamine (Cₘₐₓ, AUC₀₋ₜ, AUC_{0-24 h}, and AUC_{0-inf}) across the dose range was dose proportional.

Across the dose range of R(−)-ketamine (5 mg, 15 mg, 30 mg, 60 mg, 100 mg, and 150 mg), the increase in exposure of 6-hydroxynorketamine was dose proportional for Cₘₐₓ, AUC₀₋ₜ, AUC_{0-24 h}, and AUC_{0-inf} with slope estimates (1) and 90% CI of 0.97 (0.9 to 1.04), 0.97 (0.9 to 1.03), and 0.97 (0.9 to 1.03), respectively. The graphical evaluation also suggests exposure of 6-hydroxynorketamine (Cₘₐₓ, AUC₀₋ₜ. AUC_{0-24 h}) across the dose range was dose proportional. Across the dose range of R(−)-ketamine (5 mg, 15 mg, 30 mg, 60 mg, 100 mg, and 150 mg), the increase in exposure of dehydronorketamine was higher than dose proportional for Cₘₐₓ, AUC₀₋ₜ, AUC_{0-24 h}, and AUC_{0-inf} with slope estimates (β1) and 90% CI of 1.28 (1.15 to 1.42), 1.45 (1.32 to 1.58), 1.31 (1.17 to 1.46), and 1.61 (1.26 to 1.95), respectively. However, based on the graphical evaluation, the exposure of dehydronorketamine (Cₘₐₓ, AUC₀₋ₜ, AUC_{0-24 h}, and AUC_{0-inf}) across the dose range, except at the highest dose (150 mg) for which it was more than dose proportional, suggest the overall exposure was approximately dose proportional. It should be noted that due to small sample size and inter-individual variability, these data are suggestive only.

### Discussion of Pharmacokinetics

The PK analysis of plasma concentrations of R(−)-ketamine following IV infusion demonstrated that, regardless of the dose level, R(−)-ketamine concentrations were quantifiable in all subjects 5 minutes after the start of the infusion and a median plasma Tₘₐₓ of range of 15 minutes to 40 minutes. The known metabolites of R(−)-ketamine were also quantifiable within 15 minutes of the start of the infusion:
norketamine was quantifiable in all subjects at 10 minutes to 15 minutes with a median plasma Tₘₐₓ range of 40 minutes to 2.00 hours
6-hydroxynorketamine was quantifiable in all subjects at 10 minutes to 15 minutes with a median plasma Tₘₐₓ range of 4.00 to 8.00 hours
dehydronorketamine was quantifiable in all subjects at 15 minutes to 1.50 hours with a median plasma Tₘₐₓ range of 1.50 to 3.00 hr.

There was a 23.0-fold (33.9 ng/mL compared to 780 ng/ml), 31.6-fold (11.4 ng/ml compared to 360 ng/ml), 27.6-fold (5.98 ng/mL compared to 165 ng/ml), and 179.8-fold (198 pg/mL compared to 35600 pg/mL) increase in arithmetic mean Cₘₐₓ for R(−)-ketamine, norketamine, 6-hydroxynorketamine, and dehydronorketamine, respectively, for 5 mg to 150 mg R(−)-ketamine dosing (for a 30-fold increase in R(−)-ketamine dose). There was a 27.1-fold (63.5 hr*ng/ml compared to 1720 hr*ng/ml), 39.4-fold (79.6 hr*ng/mL compared to 3140 hr*ng/ml), 27.3-fold (110 hr*ng/mL compared to 3000 hr*ng/ml), and 169.6-fold (2500 hr*pg/mL compared to 424000 hr*pg/mL) increase in arithmetic mean AUC_{0-24 h} for R(−)-ketamine, norketamine, 6-hydroxynorketamine, and dehydronorketamine, respectively, for 5 mg to 150 mg R(−)-ketamine dosing (for a 30-fold increase in R(−)-ketamine dose). There was a 29.9-fold (57.5 hr*ng/mL compared to 1720 hr*ng/ml), 40.3-fold (78.0 hr*ng/mL compared to 3140 hr*ng/ml), 27.3-fold (110 hr*ng/mL compared to 3000 hr*ng/ml), and 309.5-fold (1370 hr*pg/mL compared to 424000 hr*pg/mL) increase in arithmetic mean AUC₀₋ₜ for R(−)-ketamine, norketamine, 6-hydroxynorketamine, and dehydronorketamine, respectively, R(−)-ketamine 1 (for a 30-fold increase in R(−)-ketamine 1 dose).

Overall, based on the statistical analysis, the plasma exposure of R(−)-ketamine and 2 metabolites (norketamine and 6-hydroxynorketamine) were dose proportional as indicated by Cₘₐₓ and AUC (AUC₀₋ₜ, AUC_{0-24 h}, AUC_{0-inf}) which increased with dose administered across all dose cohorts. Based on the graphical evaluation, the exposure of dehydronorketamine (Cₘₐₓ, AUC₀₋ₜ, AUC_{0-24 h}, and AUC_{0-inf}) across the dose range suggest the overall exposure was approximately dose proportional, which the exception of the highest dose of R(−)-ketamine (150 mg) for which it was more than dose proportional. It should be noted that due to small sample size, inter-individual variability and considering dehydronorketamine was not stable in the samples, these data are suggestive only.

Overall, inter-individual variability was low to moderate for Cₘₐₓ, AUC₀₋ₜ, AUC_{0-24 h}, and AUC_{0-inf} with geometric CV % values ranging from 13.3% to 30.3%, 12.1% to 28.4%, 12.8% to 46.0%, and 11.1% to 51.5% for R(−)-ketamine, norketamine, 6-hydroxynorketamine, and dehydronorketamine, respectively.

Overall, arithmetic mean (CV %) CL and Vz of plasma R(−)-ketamine were approximately consistent across all dose cohorts and were in the range of 76.8 to 97.5 L/hr (13.7% to 22.6%) and 345 to 921 L (20.0% to 50.2%), respectively. Following the attainment of peak plasma concentrations, a monophasic decline was observed with arithmetic mean t_{1/2} range of 3.06 to 7.21 hours of R(−)-ketamine 1, 6.70 to 8.05 hours for norketamine, and 7.41 to 9.32 hours for dehydronorketamine across all dose cohorts. The t_{1/2} was NC for 6-hydroxynorketamine due to inadequate concentration data in the elimination phase.

Overall, the arithmetic mean (CV %) Cmax metabolite to the parent (R(−)-ketamine) ratio was higher for norketamine and lower for dehydronorketamine with a range of 0.00581 to 0.448 (19.3% to 90.0%) across all R(−)-ketamine dose cohorts. Overall, the arithmetic mean (CV %) AUC0-t and AUC_{0-24 h} metabolite to parent (R(−)-ketamine) ratio were higher for 6-hydroxynorketamine and lower for dehydronorketamine with a range of 0.0223 to 2.22 (8.7% to 63.3%) and 0.0512 to 2.22 (8.7% to 63.3%), respectively, across all dose cohorts. Overall, the arithmetic mean (CV %) AUC_{0-inf} metabolite to parent (R(−)-ketamine) ratio was higher for norketamine compared to dehydronorketamine with a range of 0.0585 to 1.88 (17.0% to 28.0%) across all dose cohorts.

The pharmacokinetics of the IV administration of R(−)-ketamine (5 mg, 15 mg, 30 mg, 60 mg, 100 mg, and 150 mg) in healthy human subjects showed:
R(−)-ketamine, norketamine, 6-hydroxynorketamine, and dehydronorketamine reached peak plasma concentrations following IV administration (infusion in 40 minutes) with a median Tmax range of approximately 15 minutes to 40 minutes, 40 minutes to 2 hours, 4 hours to 8 hours, and 1.50 to 3 hours across all R(−)-ketamine dose cohorts, respectively.
Following the IV administration of R(−)-ketamine, the rate and extent of systemic exposure of R(−)-ketamine and its metabolites (norketamine, 6-hydroxynorketamine, and dehydronorketamine) as measured by Cmax and AUC (AUC₀₋ₜ, AUC_{0-24 h}, AUC_{0-inf}) appeared to be approximately dose proportional over a 30-fold R(−)-ketamine dose range (5 mg to 150 mg).
Terminal half-life (t_{1/2}) estimates were low to moderate across all dose cohorts for R(−)-ketamine and 2 metabolites (norketamine and dehydronorketamine): 3.0 to 9.5 hours. Clearance and terminal volume of distribution of R(−)-ketamine were in the range of 76.8 to 97.5 L/hr and 345 to 921 L, respectively, and were comparable across all dose cohorts.
Metabolite to parent (R(−)-ketamine) ratio of Cmax (0.448) was relatively higher for norketamine and AUC (AUC₀₋ₜ: 2.22 and AUC_{0-24 h}: 2.22) was relatively higher for 6-hydroxynorketamine compared to other metabolites across all dose cohorts.

### CITATION LIST

### Non-Patent Literature

[NPL 1] Janssen Pharmaceutical Companies. Medication Guide SPRAVATO™ CIII (esketamine) nasal spray: prescribing information. 2020. Titusville, NJ, USA.
[NPL 2] Yang C, Shirayama Y, Zhang J C, et al. R-ketamine: a rapid-onset and sustained antidepressant without psychotomimetic side effects. Transl Psychiatry, 2015; 5:e632.
[NPL 3] Tian Z, Dong C, Fujita A, et al. Expression of heat shock protein HSP-70 in the retrosplenial cortex of rat brain after administration of (R,S)-ketamine and(S)-ketamine, but not (R)-ketamine. Pharmacol Biochem Behav, 2018; 172:17-21.
[NPL 4] Hashimoto K, Kakiuchi T, Ohba H, et al. Reduction of dopamine D2/3 receptor binding in the striatum after a single administration of esketamine, but not R-ketamine: a PET study in conscious monkeys. Eur Arch Psychiatry Clin Neurosci, 2017; 267(2):173-176.
[NPL 5] Tian Z, Dong C, Zhang K, et al. Lack of antidepressant effects of (2R,6R)-hydroxynorketamine in a rat learned helplessness model: comparison with (R)-ketamine. Int J Neuropsychopharmacol, 2018; 21(1):84-88.
[NPL 6] Ide S, Ikekubo Y, Mishina M, et al. Cognitive impairment that is induced by (R)-ketamine is abolished in NMDA GluN2D receptor subunit knockout mice. Int J Neuropsychopharmacol, 2019; 22(7):449-452.
[NPL 7] Chang L, Zhang K, Pu Y, et al. Comparison of antidepressant and side effects in mice after intranasal administration of (R,S)-ketamine, (R)-ketamine, and (S)-ketamine. Pharmacol Biochem Behav, 2019; 181:53-59.
[NPL 8] Chang L, Zhang K, Pu Y, et al. Lack of dopamine D1 receptors in the antidepressant actions of (R)-ketamine in a chronic social defeat stress model. Eur Arch Psychiatry Clin Neurosci, 2020; 270:271-275.
[NPL 9] Ago Y, Tanabe W, Higuchi M, et al. (R)-ketamine induces a greater increase in prefrontal 5-HT release than(S)-ketamine and ketamine metabolites via an AMPA receptor-independent mechanism. Int J Neuropsychopharmacol, 2019; 22(10):665-674.
[NPL 10] Fujita A, Fujita Y, Pu Y, et al. MPTP-induced dopaminergic neurotoxicity in mouse brain is attenuated after subsequent intranasal administration of (R)-ketamine: a role of TrkB signaling. Psychopharmacology (Berl), 2019; 237(1):83-92.
[NPL 11] Fukumoto K, Toki H, Iijima M, et al. Antidepressant potential of (R)-ketamine in rodent models: comparison with(S)-ketamine. J Pharmacol Exp Ther, 2017; 361(1):9-16.
[NPL 12] Yang C, Qu Y, Abe M, et al. (R)-ketamine shows greater potency and longer lasting antidepressant effects than its metabolite (2R,6R)-hydroxynorketamine. Biol Psychiatry, 2017; 82(5):e43-e44.
[NPL 13] Yang C, Qu Y, Fujita Y, et al. Possible role of the gut microbiota-brain axis in the antidepressant effects of (R)-ketamine in a social defeat stress model. Transl Psychiatry, 2017; 7(12):1294.
[NPL 14] Zhang J C, Li S X, Hashimoto K. R(−)-ketamine shows greater potency and longer lasting antidepressant effects than S(+)-ketamine. Pharmacol Biochem Behav, 2014; 116:137-141.
[NPL 15] Shirayama Y, Hashimoto K. Effects of a single bilateral infusion of R-ketamine in the rat brain regions of a learned helplessness model of depression. Eur Arch Psychiatry Clin Neurosci, 2017; 267(2):177-182.
[NPL 16] Yang C, Kobayashi S, Nakao K, et al. AMPA n(S)-Norketamine. Biol Psychiatry, 2018; 84(8):591-600.
[NPL 17] Li J M, Liu L L, Su W J, et al. Ketamine may exert antidepressant effects via suppressing NLRP3 inflammasome to upregulate AMPA receptors. Neuropharmacology, 2019; 146:149-153.
[NPL 18] Zhang M, Radford K D, Driscoll M, et al. Effects of subanesthetic intravenous ketamine infusion on neuroplasticity-related proteins in the prefrontal cortex, amygdala, and hippocampus of Sprague-Dawley rats. IBRO Rep, 2019; 6:87-94.
[NPL 19] Zanos P, Highland J N, Liu X, et al. (R)-ketamine exerts antidepressant actions partly via conversion to (2R,6R)-hydroxynorketamine, while causing adverse effects at sub-anesthetic doses. Br J Pharmacol, 2019; 176(14):2573-2592.
[NPL 20] Vollenweider F X, Leenders K L, Oye I, et al. Differential psychopathology and patterns of cerebral glucose utilisation produced by (S)- and (R)-ketamine in healthy volunteers using positron emission tomography (PET). Eur Neuropsychopharmacol 1997; 7(1):25-38.
[NPL 21] Klepstad P, Maurset A, Moberg E R, Oye I. Evidence of a role for NMDA receptors in pain perception. Eur J Pharmacol, 1990; 187(3):513-518.
[NPL 22] Halder P. Effects of S- and R-ketamine on the Mismatch Negativity Evet Related Potential: Implications for Schizophrenia. In Psychiatric University Hospital Zurich, Switzerland, Behavioural Neurobiology Laboratory, Swiss Federal Institute of Technology, 1999; 88.
[NPL 23] Mathisen L C, Skjelbred P, Skoglund L A, Oye I. Effect of ketamine, an NMDA receptor inhibitor, in acute and chronic orofacial pain. Pain, 1995; 61(2):215-220.
[NPL 24] Oye I, Paulsen O, Maurset A. Effects of ketamine on sensory perception: evidence for a role of N-methyl-D-aspartate receptors. J Pharmacol Exp Ther, 1992; 260(3):1209-1213.
[NPL 25] Pfenninger E G, Durieux M E, Himmelseher S. Cognitive impairment after small-dose ketamine isomers in comparison to equianalgesic racemic ketamine in human volunteers. Anesthesiology, 2002; 96(2):357-66. WHO. Depression. 2017; Available from: www.who.int/news-room/fact-sheets/detail/depression.
[NPL 26] WHO. Depression. 2017; Available from: www.who.int/news-room/fact-sheets/detail/depression.
[NPL 27] CDC. National Violent Death Reporting System. 2015; Available from: www.cdc.gov/violenceprevention/nvdrs/index.html.
[NPL 28] Canuso C M, Singh J B, Fedgchin M, et al. Efficacy and safety of intranasal esketamine for the rapid reduction of symptoms of depression and suicidality in patients at imminent risk for suicide: results of a double-blind, randomized, placebo-controlled study. Am J Psychiatry, 2018; 175(7):620-630.
[NPL 29] Souery D, Oswald P, Massat I, et al. Clinical factors associated with treatment resistance in major depressive disorder: results from a European multicenter study. J Clin Psychiatry, 2007; 68(7):1062-1070.
[NPL 30] Ivanova J I, Birnbaum H, Kidolezi Y, et al. Direct and indirect costs of employees with treatment-resistant and non-treatment-resistant major depressive disorder. Curr Med Res Opin, 2010; 26(10):2475-2484.
[NPL 31] Chan W H, Sun W Z, Ueng T H. Induction of rat hepatic cytochrome P-450 by ketamine and its toxicological implications. J Toxicol Environ Health A, 2005; 68(17-18):1581-1597.
[NPL 32] Zanos P, Gould T D. Intracellular signaling pathways involved in (S)- and (R)-ketamine antidepressant actions. Biol Psychiatry, 2018; 83(1):2-4.
[NPL 33] Zanos P, Moaddel R, Morris P J, et al. Ketamine and ketamine metabolite pharmacology: insights into therapeutic mechanisms. Pharmacol Rev, 2018; 70(3):621-660.
[NPL 34] White P F, Schüttler J, Shafer A, et al. Comparative pharmacology of the ketamine isomers. Studies in volunteers. Br J Anaesth, 1985; 57(2):197-203.
[NPL 35] Leal G C, Bandeira I D, Correia-Melo, F S, et al. Intravenous arketamine for treatment-resistant depression: open-label pilot study. Eu Arch Psych Clin Neurosci 20 Feb. 2020. doi: 10.1007/s00406-020-01110-5.
[NPL 36] Singh J B, Fedgchin M, Daly E J, et al. Intravenous Esketamine in Adult Treatment-Resistant Depression: A Double-Blind, Double-Randomization, Placebo-Controlled Study. Biological Psychiatry 2016; September 15; 80(6):424-431.
[NPL 37] Sheehan D V, Lecrubier Y, Sheehan K H, et al. The Mini-International Neuropsychiatric Interview (M.I.N.I.): the development and validation of a structured diagnostic psychiatric interview for DSM-IV and ICD-10. J Clin Psychiatry 1998; 59 Suppl 20:22-33; quiz 34-57.
[NPL 38] Jovaisa T, et al. Effects of ketamine on precipitated opiate withdrawal. Medicina (Kaunas), 42(8):625-634 (2006).
[NPL 39] Herman B H, et al. The Effects of NMDA Receptor Antagonists and Nitric Oxide Synthase Inhibitors on Opioid Tolerance and Withdrawal Medication Development Issues for Opiate Addiction. Neuropsychopharmacology, 13(4):269-293 (1995).
[NPL 40] Khanna J M, et al. Effect of NMDA receptor antagonists on rapid tolerance to ethanol. European Journal of Pharmacology, 230:23-31 (1993).
[NPL 41] Trujillo K A, Effects of Noncompetitive N-Methyl-D-Aspartate Receptor Antagonists on Opiate Tolerance and Physical Dependence. Neuropsychopharmacology, 13:301-307 (1995).
[NPL 42] Cooper M D, et al. Strategies to mitigate dissociative and psychotomimetic effects of ketamine in the treatment of major depressive episodes: a narrative review. The World Journal of Biological Psychiatry, 18:6, 410-423 (2017).
[NPL 43] Ke X, et al. The profile of cognitive impairments in chronic ketamine users. Psychiatry Research, 266, 124-131 (2018).
[NPL 44] Liu Y, et al. Ketamine abuse potential and use disorder. Brain Research Bulletin, 126:68-73 (2016).
[NPL 45] Wang C, et al. Brain damages in ketamine addicts as revealed by magnetic resonance imaging. Front. Neuroanat., Volume 7, Article 23 (2013).

What is claimed is:

1. A method of treating or ameliorating a depressive symptom of a subject, the method comprising administering a therapeutically effective amount of a composition comprising R(−)-ketamine or a pharmaceutically acceptable salt thereof to the subject,
wherein the composition is substantially free of S(+)-ketamine or a pharmaceutically acceptable salt thereof, and
wherein the therapeutically effective amount comprises a maximum blood plasma concentration (Cmax) of R(−)-ketamine of at least 250 ng/mL.

2. The method of claim 1, wherein the Cmax is between 250 and 1000 ng/ml.

3. The method of claim 1, wherein the Cmax is between 250 and 800 ng/mL.

4. The method of claim 1, wherein the depressive symptom is assessed by a Montgomery Åsberg Depression Rating Scale (MADRS) subject score, and
wherein the MADRS subject score is decreased by administration of the composition.

5. The method of claim 4, wherein the MADRS subject score is decreased by about 2 to about 20 by administration of the composition.

6. The method of claim 4, wherein the MADRS subject score is decreased by about 2 to about 20 when measured about 24 hours, about 7 days, or about 14 days after administration of the composition begins.

7. The method of claim 4, wherein the MADRS subject score is decreased by about 2 to about 20 after administration of the composition begins.

8. The method of claim 1, wherein the therapeutically effective dose of the composition comprises from about 50 mg to about 150 mg R(−)-ketamine or a pharmaceutically acceptable salt thereof.

9. The method of claim 1, wherein the composition comprises R(−)-ketamine hydrochloride.

10. The method of claim 1, wherein the composition is administered intravenously or subcutaneously.

11. The method of claim 10, wherein the intravenous administration comprises intravenous infusion.

12. The method of claim 11, wherein the composition is administered over a time period from about 10 minutes to about 1.5 hours.

13. The method of claim 1, wherein the composition is administered once per day, one every other day, once every three days, once every four days, once every 5 days, once every 6 days, once per week, every other week, every 10 days, or once per month.

14. The method of claim 1, comprising a dosing schedule comprising:
(a) an initial period in which the composition is administered every 1, 2, 3 or 4 days; and
(b) a maintenance period in which composition is administered less frequently than during the initial period.

15. The method of claim 14, wherein the composition is administered once per week, twice per week, every other week, every 10 days, or once per month during the maintenance period.

16. The method of claim 1, wherein composition further comprises a pharmaceutically acceptable carrier.

17. The method of claim 1, wherein the depressive symptom is a symptom of a mood disorder in the subject.

18. The method of claim 17, wherein the mood disorder comprises depression, optionally wherein the depression is treatment resistant depression or major depressive disorder.

19. The method of claim 17, wherein the mood disorder comprises bipolar disorder, post traumatic stress disorder, obsessive compulsive disorder, autism spectrum disorder, schizophrenia, or dementia.

20. The method of claim 1, wherein the depressive symptom is associated with a substance use disorder in the subject.

21. The method of claim 1, wherein the therapeutically effective amount of the composition comprising R(−)-ketamine does not cause significant dissociation, derealization or sedation in the subject.

22. The method of claim 1, wherein the therapeutically effective amount of the composition increases systolic blood pressure of the subject by less than 40 mmHg, optionally less than 10 mmHg, as measured within 14 days of administration.

23. The method of claim 1, wherein the therapeutically effective amount of the composition increases diastolic blood pressure of the subject by less than 25 mmHg, optionally less than 10 mmHg, as measured within 14 days of administration.

24. The method of claim 1, wherein administration of the therapeutically effective amount of the composition has fewer side effects or adverse events than administration of a therapeutically effective amount of S(+)-ketamine or racemic ketamine.

25. A method of disease or disorder in a subject, the method comprising administering a therapeutically effective amount of a composition comprising R(−)-ketamine or a pharmaceutically acceptable salt thereof to the subject,
wherein the composition is substantially free of S(+)-ketamine or a pharmaceutically acceptable salt thereof, and
wherein the therapeutically effective amount comprises a maximum blood plasma concentration (Cmax) of R(−)-ketamine of at least 250 ng/mL.

26. The method of claim 25, wherein the Cmax is between 250 and 1000 ng/ml.

27. The method of claim 25, wherein the Cmax is between 250 and 800 ng/mL.

28. The method of claim 25, wherein the composition comprises R(−)-ketamine hydrochloride.

29. The method of claim 25, wherein the composition is administered intravenously or subcutaneously.

30. The method of claim 29, wherein the intravenous administration comprises intravenous infusion.

31. The method of claim 30, wherein the composition is administered over a time period from about 10 minutes to about 1.5 hours.

32. The method of claim 25, wherein the composition is administered once per day, one every other day, once every three days, once every four days, once every 5 days, once every 6 days, once per week, every other week, every 10 days, or once per month.

33. The method of claim 25, comprising a dosing schedule comprising:
(a) an initial period in which the composition is administered every 1, 2, 3 or 4 days; and
(b) a maintenance period in which composition is administered less frequently than during the initial period.

34. The method of claim 33, wherein the composition is administered once per week, twice per week, every other week, every 10 days, or once per month during the maintenance period.

35. The method of claim 25, wherein composition further comprises a pharmaceutically acceptable carrier.

36. The method of claim 25, wherein the disease or disorder is a neurodegenerative disease or disorder, a neurodevelopmental disorder, an inflammatory or bone disease.

37. The method of claim 36, wherein the neurodegenerative disease or disorder comprises Parkinson's disease, Parkinson's syndrome, Huntington's disease, spiny erythrocyte chorea, spinal cord cerebellar degeneration, amyotrophic lateral sclerosis, spinal muscular atrophy, primary lateral sclerosis, spinal and bulbar muscular atrophy, syringomyelia, neurospinous erythrocytosis, eating disorders, Alzheimer's disease, Lewy body dementia, basal ganglia degeneration, multiple sclerosis, traumatic brain injury, cerebral infarction, or cardiovascular disease.

38. The method of claim 36, wherein the neurodevelopmental disorder comprises schizophrenia, autism spectrum disorder, attention-deficit/hyperactivity disorder, or a learning disorder.

39. The method of claim 36, wherein the inflammatory disease comprises ulcerative colitis, Crohn's disease, rheumatoid arthritis, ankylosing spondylitis, insulin-dependent diabetes, Addison's disease, Goodpasture syndrome, IgA nephropathy, interstitial nephritis, Sjögren's syndrome, autoimmune pancreatitis, psoriasis, atopic dermatitis, pneumonia, chronic bronchitis, bronchial asthma, systemic lupus erythematosus (SLE), scleroderma, or delirium, and the bone disease comprises osteoporosis, osteolytic bone metastasis, or Paget's disease of bone.

40. The method of claim 25, wherein the therapeutically effective amount of the composition comprising R(−)-ketamine does not cause significant dissociation, derealization or sedation in the subject.

41. The method of claim 25, wherein the therapeutically effective amount of the composition increases systolic blood pressure of the subject by less than 40 mmHg, optionally less than 10 mmHg, as measured within 14 days of administration.

42. The method of claim 25, wherein the therapeutically effective amount of the composition increases diastolic blood pressure of the subject by less than 25 mmHg, optionally less than 10 mmHg, as measured within 14 days of administration.

43. The method of claim 25, wherein administration of the therapeutically effective amount of the composition has fewer side effects or adverse events than administration of a therapeutically effective amount of S(+)-ketamine or racemic ketamine.
